(19) United States
(12) Patent Application Publication
(10) Pub. No.: US 2022/0080189 A1
(43) Pub. Date: Mar. 17, 2022

(54) STIMULATION ENERGY SYSTEMS WITH CURRENT STEERING

(71) Applicant: Nalu Medical, Inc., Carlsbad, CA (US)

(72) Inventors: Lakshmi Narayan MISHRA, Carlsbad, CA (US); Mandar Hemant GADGIL, Pune (IN); Gaurav Gajanan KULKARNI, Pune (IN); Lee Fason HARTLEY, Carlsbad, CA (US); Chaitanya Shrikrishna GUTTIKAR, Pune (IN)

(21) Appl. No.: 17/383,985

(22) Filed: Jul. 23, 2021

Related U.S. Application Data
(63) Continuation of application No. PCT/US21/38545, filed on Jun. 22, 2021.
(60) Provisional application No. 63/042,293, filed on Jun. 22, 2020.
(60) Provisional application No. 63/082,856, filed on Sep. 24, 2020.

(51) Int. Cl. A61N 1/05; A61N 1/372; A61N 1/36

(52) U.S. Cl. CPC A61N 1/0551; A61N 1/36125; A61N 1/37252

(57) ABSTRACT

A system for delivering stimulation energy to a patient is provided. The system comprises a controller, a memory coupled to the controller and storing instructions for the controller to perform an algorithm, and one or more leads for implantation inside a human body. Each lead comprises a plurality of stimulation elements, and each stimulation element can be in one of at least two configuration states. A first configuration state comprises a stimulation element sourcing and/or sinking current, and a second configuration state comprises a stimulation element being in an electrically passive state. The algorithm is configured to determine a stimulation paradigm for stimulating one or more target locations of a patient. The stimulation paradigm defines the configuration state of each of the plurality of the stimulation elements of a first lead. Methods of delivering stimulation energy is also provided.

### CROSS REFERENCE TO RELATED APPLICATIONS

This application is a continuation of PCT Application No. PCT/US21/38545, filed Jun. 22, 2021; which claims priority to U.S. Provisional Application No. 63/042,293, filed Jun. 22, 2020; and U.S. Provisional Application No. 63/082,856, filed Sep. 24, 2020; the contents of which are incorporated herein by reference in their entirety for all purposes.

The subject matter of this application is related to that in: U.S. patent application Ser. No. 14/975,358, titled “Method and Apparatus for Minimally Invasive Implantable Modulators”, filed Dec. 18, 2015 [Docket nos. 47476-703.301; NAL-005-US]; U.S. patent application Ser. No. 15/664,231, titled “Medical Apparatus Including an Implantable System and an External System”, filed Jul. 31, 2017 [Docket nos. 47476-706.301; NAL-011-US]; U.S. patent application Ser. No. 16/104,829, titled “Apparatus with Enhanced Stimulation Waveforms”, filed Aug. 17, 2018 [Docket nos. 47476-708.301; NAL-014-US]; U.S. patent application Ser. No. 16/111,868, titled “Devices and Methods for Positioning External Devices in Relation to Implanted Devices”, filed Aug. 24, 2018 [Docket nos. 47476-709.301; NAL-016-US]; U.S. patent application Ser. No. 16/222,959, titled “Methods and Systems for Treating Pelvic Disorders and Pain Conditions”, filed Dec. 17, 2018 [Docket nos. 47476-711.301; NAL-017-US]; U.S. patent application Ser. No. 16/266,822, titled “Method and Apparatus for Versatile Minimally Invasive Neuromodulators”, filed Feb. 4, 2019 [Docket nos. 47476-704.302; NAL-007-US-CON1]; U.S. patent application Ser. No. 16/453,917, titled “Stimulation Apparatus”, filed Jun. 26, 2019 [Docket nos. 47476-712.301; NAL-015-US]; U.S. patent application Ser. No. 16/505,425, titled “Wireless Implantable Sensing Devices”, filed Jul. 8, 2019 [Docket nos. 10220-728.300; NAL-006-US-CON1]; U.S. patent application Ser. No. 16/539,977, titled “Apparatus with Sequentially Implanted Stimulators”, filed Aug. 13, 2019 [Docket nos. 47476-713.301; NAL-019-US]; U.S. patent application Ser. No. 16/672,921, titled “Stimulation Apparatus”, filed Nov. 4, 2019 [Docket nos. 47476-714.301; NAL-020-US]; International PCT Patent Application Serial Number PCT/US2020/040766, titled “Stimulation Apparatus”, filed Jul. 2, 2020 [Docket nos. 47476-715.601; NAL-021-PCT]; U.S. patent application Ser. No. 16/993,999, titled “Apparatus for Peripheral or Spinal Stimulation”, filed Aug. 14, 2020 [Docket nos. 47476-707.302; NAL-012-US-CON1]; U.S. Provisional Application Ser. No. 63/071,925, titled “Apparatus for Delivering Customized Stimulation Waveforms”, filed Aug. 28, 2020 [Docket Nos. 47476-718.101; NAL-024-PR1]; International PCT Patent Application Serial Number PCT/US2020/054150, titled “Stimulation Apparatus”, filed Oct. 2, 2020 [Docket Nos. 47476-719.601; NAL-025-PCT]; U.S. patent application Ser. No. 17/081,351, titled “Methods and Systems for Insertion and Fixation of Implantable Devices”, filed Oct. 27, 2020 [Docket nos. 47476-710.302; NAL-013-US-CON1]; U.S. Provisional Patent Application Ser. No. 63/112,055, titled “Apparatus for Delivering Enhanced Stimulation Waveforms”, filed Nov. 10, 2020 [Docket nos. 47476-723.101; NAL-026-PR1]; International PCT Patent Application Serial Number PCT/US2020/066901, titled “Systems with Implanted Conduit Tracking”, filed Dec. 23, 2020 [Docket nos. 47476-716.601; NAL-022-PCT]; U.S. patent application Ser. No. 17/187,654, titled “Method and Apparatus for Neuromodulation Treatments of Pain and Other Conditions”, filed Feb. 26, 2021 [Docket nos. 47476-705.303; NAL-008-US-CON2]; U.S. Provisional Patent Application Ser. No. 63/161,757, titled “Apparatus for Delivering Enhanced Stimulation Waveforms”, filed Mar. 16, 2021 [Docket nos. 47476-724.101; NAL-027-PR1]; and U.S. patent application Ser. No. 17/240,629, titled “Method and Apparatus for Minimally Invasive Implantable Modulators”, filed Apr. 26, 2021 [Docket nos. 47476-703.302; NAL-005-US-CON1]; the contents of each of which is incorporated herein by reference in its entirety for all purposes.

### TECHNICAL FIELD

The present invention relates generally to medical apparatus for a patient, and in particular, systems including implantable conduits for delivering steerable stimulation energy.

### BACKGROUND OF THE INVENTION

Delivery devices that treat a patient and/or record patient data are known. For example, implants and other delivery devices that deliver energy, such as electrical energy, or deliver agents such as pharmaceutical agents are commercially available. Electrical stimulators can be used to pace or defibrillate the heart, as well as modulate nerve tissue (e.g. to treat pain). Most implants are relatively large devices with batteries and long conduits, such as implantable leads configured to deliver electrical energy or implantable tubes (i.e. catheters) to deliver an agent. These implants require a fairly invasive implantation procedure, and periodic battery replacement, which requires additional surgery. The large sizes of these devices and their high costs have prevented their use in a variety of applications.

Nerve stimulation treatments have shown increasing promise recently, showing potential in the treatment of many chronic diseases including drug-resistant hypertension, motility disorders in the intestinal system, metabolic disorders arising from diabetes and obesity, and both chronic and acute pain conditions among others. Many of these delivery device configurations have not been developed effectively because of the lack of miniaturization and power efficiency, in addition to other limitations. For example, migration of implanted components can lead to compromised results.

There is a need for apparatus, systems, devices and methods that provide one or more therapy delivering devices and are designed to provide enhanced therapy and other enhanced benefits.

### SUMMARY

According to an aspect of the present inventive concepts, a system for delivering stimulation energy to a patient comprises: a controller, a memory coupled to the controller storing instructions for the controller to perform an algorithm, and one or more leads for implantation inside a human body. Each lead comprises a plurality of stimulation elements, and each stimulation element can be in one of at least two configuration states. A first configuration state comprises a stimulation element sourcing and/or sinking current, and a second configuration state comprises a stimulation element being in an electrically passive state. The algorithm is configured to determine a stimulation paradigm for stimulating one or more target locations of a patient. The stimulation paradigm defines the configuration state of each of the plurality of the stimulation elements of a first lead.

In some embodiments, the stimulation paradigm further defines the configuration state of each of the plurality of the stimulation elements of a second lead.

In some embodiments, the plurality of stimulation elements of each lead comprises four or more stimulation elements, and the algorithm is configured to cause four of the stimulation elements of each lead to source and/or sink current, and the algorithm is further configured to cause the remaining stimulation elements of each lead to be in a floating state.

In some embodiments, the first configuration comprises: a first arrangement in which a first set of one or more stimulation elements each deliver a particular current; and/or a second arrangement in which a second set of one or more stimulation elements are each set to a particular voltage. The first arrangement can comprise the first set of stimulation elements each delivering a positive or zero current, and the second arrangement can comprise the second set of stimulation elements each being set to ground voltage. The first arrangement can comprise the first set of stimulation elements each delivering a negative or zero current, and the second arrangement can comprise the second set of stimulation elements each being set to ground voltage. In some embodiments, no greater than four of the stimulation elements of each of the one or more leads are configured in the first arrangement.

In some embodiments, the algorithm is configured to determine the stimulation paradigm that preferentially stimulates a target region by maximizing the ratio of the integral of the square of the current density in the target region to the integral of the square of the current density in a large part of the human body. The algorithm can determine the stimulation paradigm by solving one or more generalized eigenvalue problems. In some embodiments, the algorithm does not match a specific current or voltage distribution through an entire domain.

In some embodiments, the algorithm is configured to steer current using a finite element analysis technique.

In some embodiments, the algorithm utilizes one or more constraints to determine the stimulation paradigm. The algorithm can limit the number of stimulation elements that are configured as a source and/or a sink.

In some embodiments, the algorithm determines the stimulation paradigm based on one or more of the stimulation elements being classified as open and/or shorted.

In some embodiments, the system further comprises a library of predetermined information, and the algorithm is configured to determine the stimulation paradigm based on the predetermined information. The algorithm can be further configured to correlate implant locations and/or implant geometries of the one or more leads to the predetermined information.

In some embodiments, the system further comprises a user interface configured to allow an operator to specify a location into which current delivered by the stimulation elements can be steered.

In some embodiments, the algorithm is configured to determine the stimulation paradigm using an inverse solution. The inverse solution can predict an anatomical location to be stimulated based on a given stimulation paradigm. The given stimulation paradigm can comprise a set of stimulation elements to be configured as anodes and/or cathodes, as well as a set of current amplitudes to be delivered to each stimulation element.

In some embodiments, the algorithm is configured to steer current to a target location in the patient's spinal cord.

In some embodiments, the algorithm is configured to steer current to a target location in the patient's peripheral nervous system.

The technology described herein, along with the attributes and attendant advantages thereof, will best be appreciated and understood in view of the following detailed description taken in conjunction with the accompanying drawings in which representative embodiments are described by way of example.

### CORPORATION BY REFERENCE

All publications, patents, and patent applications mentioned in this specification are herein incorporated by reference to the same extent as if each individual publication, patent, or patent application was specifically and individually indicated to be incorporated by reference.

The content of all publications, patents, and patent applications mentioned in this specification are herein incorporated by reference in their entirety for all purposes.

### BRIEF DESCRIPTION OF THE DRAWINGS

The foregoing and other objects, features and advantages of embodiments of the present inventive concepts will be apparent from the more particular description of preferred embodiments, as illustrated in the accompanying drawings in which like reference characters refer to the same or like elements. The drawings are not necessarily to scale, emphasis instead being placed upon illustrating the principles of the preferred embodiments.

FIG. 1 is a schematic view of a medical apparatus comprising a delivery device with an implantable lead, consistent with the present inventive concepts.

FIG. 1A is a schematic anatomical view of a medical apparatus comprising an external system and an implantable system, consistent with the present inventive concepts.

FIG. 2 is a schematic of electronic circuitry configured to steer stimulation current, consistent with the present inventive concepts.

FIG. 3 is a graph of a multiplexing scheme to achieve a desired steering of current delivered by multiple stimulation elements, consistent with the present inventive concepts.

FIG. 4 is a graph of a multiplexing scheme to achieve a desired steering of current delivered by multiple stimulation elements, consistent with the present inventive concepts.

FIGS. 5A and 5B are schematics of circuitry of an implantable stimulator in which time division multiplexing is employed, consistent with the present inventive concepts.

FIGS. 6A and 6B are views of a user interface in which an operator of a stimulation system can configure multiple stimulation elements for current delivery, consistent with the present inventive concepts.

FIG. 7 is a flow chart of a method of steering current, consistent with the present inventive concepts.

FIGS. 8A and 8B are a sectional anatomical view, and a magnified sectional anatomical view, respectively, of a patient's spinal cord with implanted leads and stimulation elements, consistent with the present inventive concepts.

FIG. 9 is an anatomical view of two leads implanted in a target region to be stimulated, consistent with the present inventive concepts.

FIG. 10 is an anatomical view of a current density resulting from the implanted leads of FIG. 9, consistent with the present inventive concepts.

FIG. 11 is an anatomical view of the steered current resulting from the implanted leads of FIG. 9, consistent with the present inventive concepts.

FIG. 12 is a top view of a first lead and a second lead, where both leads are in a relative straight geometry, and where the second lead is positioned at an angle relative to the first lead, consistent with the present inventive concepts.

FIG. 13 is a top view of a first lead and a second lead, where the first lead is in a relatively straight geometry, and where different points along the second lead are at different separation distances from corresponding points along the first lead, consistent with the present inventive concepts.

FIG. 14 is a top view of a first lead and a second lead, where the first lead is in a relatively straight geometry, and where different points along the second lead are at different separation distances from corresponding points along the first lead, consistent with the present inventive concepts.

FIG. 15 is a top view of a fist lead and a second lead, where both leads are in a curvilinear geometry, consistent with the present inventive concepts.

FIG. 16 is a top view of a first lead and a second lead, where both leads are in a straight geometry and in a staggered arrangement, consistent with the present inventive concepts.

FIG. 17 is an anatomical sectional view of a patient's spinal canal with two implanted leads, consistent with the present inventive concepts.

FIGS. 18A, 18B, and 18C are each anatomical sectional views of a patient's spinal canal with two implanted leads, consistent with the present inventive concepts.

FIG. 19 is a view of a user interface of a stimulation system, consistent with the present inventive concepts.

FIG. 20 is an anatomical sectional view of a lead comprising multiple stimulation elements that has been implanted proximate a peripheral nerve, consistent with the present inventive concepts.

FIGS. 21, 22, 23, 24, 25, 26, 27, and 28 are sets of anatomical sectional and side views of one or more leads that have been implanted proximate a peripheral nerve, consistent with the present inventive concepts.

FIG. 29 is a user's view of a user interface of a stimulation system, consistent with the present inventive concepts.

FIG. 30 is two schematic views of a pair of implanted leads, and a set of three steps for determination of a stimulation paradigm, consistent with the present inventive concepts.

### DETAILED DESCRIPTION OF THE DRAWINGS

The terminology used herein is for the purpose of describing particular embodiments and is not intended to be limiting of the inventive concepts. Furthermore, embodiments of the present inventive concepts may include several novel features, no single one of which is solely responsible for its desirable attributes or which is essential to practicing an inventive concept described herein. As used herein, the singular forms “a,” “an” and “the” are intended to include the plural forms as well, unless the context clearly indicates otherwise.

It will be further understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be understood that, although the terms first, second, third etc. may be used herein to describe various limitations, elements, components, regions, layers, and/or sections, these limitations, elements, components, regions, layers, and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.). A first component (e.g. a device, assembly, housing or other component) can be “attached”, “connected” or “coupled” to another component via a connecting filament (as defined below). In some embodiments, an assembly comprising multiple components connected by one or more connecting filaments is created during a manufacturing process (e.g. pre-connected at the time of an implantation procedure of the apparatus of the present inventive concepts). Alternatively or additionally, a connecting filament can comprise one or more connectors (e.g. a connectorized filament comprising a connector on one or both ends), and a similar assembly can be created by an operator (e.g. a clinician) operably attaching the one or more connectors of the connecting filament to one or more mating connectors of one or more components of the assembly.

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of these.

Spatially relative terms, such as “beneath,” “below,” “lower,” “above,” “upper” and the like may be used to describe an element and/or feature's relationship to another element(s) and/or feature(s) as, for example, illustrated in the figures. It will be understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

As used herein, the term “proximate” shall include locations relatively close to, on, in, and/or within a referenced component or other location.

The term “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross-sectional area as the cross section of the component being described.

The terms “major axis” and “minor axis” of a component where used herein are the length and diameter, respectively, of the smallest volume hypothetical cylinder which can completely surround the component.

The term “functional element” where used herein, is the be taken to include a component comprising one, two or more of: a sensor; a transducer; an electrode; an energy delivery element; an agent delivery element; a magnetic field generating transducer; and combinations of these. In some embodiments, a functional element comprises a transducer selected from the group consisting of: light delivery element; light emitting diode; wireless transmitter; Bluetooth device; mechanical transducer; piezoelectric transducer; pressure transducer; temperature transducer; humidity transducer; vibrational transducer; audio transducer; speaker; and combinations of these. In some embodiments, a functional element comprises a needle, a catheter (e.g. a distal portion of a catheter), an iontophoretic element or a porous membrane, such as an agent delivery element configured to deliver one or more agents. In some embodiments, a functional element comprises one or more sensors selected from the group consisting of: electrode; sensor configured to record electrical activity of tissue; blood glucose sensor such as an optical blood glucose sensor; pressure sensor; blood pressure sensor; heart rate sensor; inflammation sensor; neural activity sensor; muscular activity sensor; pH sensor; strain gauge; accelerometer; gyroscope; GPS; respiration sensor; respiration rate sensor; temperature sensor; magnetic sensor; optical sensor; MEMs sensor; chemical sensor; hormone sensor; impedance sensor; tissue impedance sensor; body position sensor; body motion sensor; physical activity level sensor; perspiration sensor; patient hydration sensor; breath monitoring sensor; sleep monitoring sensor; food intake monitoring sensor; urine movement sensor; bowel movement sensor; tremor sensor; pain level sensor; orientation sensor; motion sensor; and combinations of these.

The term “transducer” where used herein is to be taken to include any component or combination of components that receives energy, or any input, and produces an output. For example, a transducer can include an electrode that receives electrical energy and distributes the electrical energy to tissue (e.g. based on the size of the electrode). In some configurations, a transducer converts an electrical signal into any output, such as light (e.g. a transducer comprising a light emitting diode or light bulb), sound (e.g. a transducer comprising a piezo crystal configured to deliver ultrasound energy), pressure, heat energy, cryogenic energy, chemical energy, mechanical energy (e.g. a transducer comprising a motor or a solenoid), magnetic energy, and/or a different electrical signal (e.g. a Bluetooth or other wireless communication element). Alternatively or additionally, a transducer can convert a physical quantity (e.g. variations in a physical quantity) into an electrical signal. A transducer can include any component that delivers energy and/or an agent to tissue, such as a transducer configured to deliver one or more of: electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising a piezo crystal); thermal energy to tissue (e.g. heat energy and/or cryogenic energy); chemical energy; electromagnetic energy; magnetic energy; and combinations of these.

The term “transmission signal” where used herein is to be taken to include any signal transmitted between two components, such as via a wired or wireless communication pathway. For example, a transmission signal can comprise a power and/or data signal wirelessly transmitted between a component external to the patient and one or more components implanted in the patient. A transmission signal can include one or more signals transmitted using body conduction. Alternatively or additionally, a transmission signal can comprise reflected energy, such as energy reflected from any power and/or data signal.

The term “data signal” where used herein is to be taken to include a transmission signal including at least data. For example, a data signal can comprise a transmission signal including data and sent between a component external to the patient and one or more components implanted in the patient. Alternatively, a data signal can comprise a transmission signal including data sent from an implanted component to one or more components external to the patient. A data signal can comprise a radiofrequency signal including data (e.g. a radiofrequency signal including both power and data) and/or a data signal sent using body conduction.

The term “implantable” where used herein is to be taken to define a component which is constructed and arranged to be fully or partially implanted in a patient's body and/or a component that has been fully or partially implanted in a patient. The term “external” where used herein is to be taken to define a component which is constructed and arranged to be positioned outside of the patient's body.

The terms “attachment”, “attached”, “attaching”, “connection”, “connected”, “connecting” and the like, where used herein, are to be taken to include any type of connection between two or more components. The connection can include an “operable connection” or “operable attachment” which allows multiple connected components to operate together such as to transfer information, power, and/or material (e.g. an agent to be delivered) between the components. An operable connection can include a physical connection, such as a physical connection including a connection between two or more: wires or other conductors (e.g. an “electrical connection”), optical fibers, wave guides, tubes such as fluid transport tubes, and/or linkages such as translatable rods or other mechanical linkages. Alternatively or additionally, an operable connection can include a non-physical or “wireless” connection, such as a wireless connection in which information and/or power is transmitted between components using electromagnetic energy. A connection can include a connection selected from the group consisting of: a wired connection; a wireless connection; an electrical connection; a mechanical connection; an optical connection; a sound propagating connection; a fluid connection; and combinations of these.

The term “connecting filament” where used herein is to be taken to define a filament connecting a first component to a second component. The connecting filament can include a connector on one or both ends, such as to allow an operator to operably attach at least one end of the filament to a component. A connecting filament can comprise one or more elements selected from the group consisting of: wires; optical fibers; fluid transport tubes; mechanical linkages; wave guides; flexible circuits; and combinations of these. A connecting filament can comprise a rigid filament, a flexible filament or it can comprise one or more flexible portions and one or more rigid portions.

The term “connectorized” where used herein is to be taken to refer to a filament, housing or other component that includes one or more connectors (e.g. clinician or other operator-attachable connectors) for operably connecting that component to a mating connector (e.g. of the same or different component).

The terms “stimulation parameter”, “stimulation signal parameter” or “stimulation waveform parameter” where used herein can be taken to refer to one or more parameters of a stimulation waveform (also referred to as a stimulation signal). Applicable stimulation parameters of the present inventive concepts shall include but are not limited to: amplitude (e.g. amplitude of voltage and/or current); average amplitude; peak amplitude; frequency; average frequency; pulse width (also referred to as “pulse pattern on time”); period; phase; polarity; pulse shape; a duty cycle parameter (e.g. frequency, pulse width, and/or off time); inter-pulse gap (also referred to as “pulse pattern off time”, or “inter-pulse interval”); polarity; burst-on (also referred to as “dosage on”) period; burst-off (also referred to as “dosage off”) period; inter-burst period; pulse train; train-on period; train-off period; inter-train period; drive impedance; duration of pulse and/or amplitude level; duration of stimulation waveform; repetition of stimulation waveform; an amplitude modulation parameter; a frequency modulation parameter; a burst parameter; a power spectral density parameter; an anode/cathode configuration parameter; amount of energy and/or power to be delivered; rate of energy and/or power delivery; time of energy delivery initiation; method of charge recovery; and combinations of these. A stimulation parameter can refer to a single stimulation pulse, multiple stimulation pulses, or a portion of a stimulation pulse. The term “amplitude” where used herein can refer to an instantaneous or continuous amplitude of one or more stimulation pulses (e.g. the instantaneous voltage level or current level of a pulse). The term “pulse” where used herein can refer to a period of time during which stimulation energy is relatively continuously being delivered. In some embodiments, stimulation energy delivered during a pulse comprises energy selected from the group consisting of: electrical energy; magnetic energy; electromagnetic energy; light energy; sound energy such as ultrasound energy; mechanical energy such as vibrational energy; thermal energy such as heat energy or cryogenic energy; chemical energy; and combinations of these. In some embodiments, stimulation energy comprises electrical energy and a pulse comprises a phase change in current and/or voltage. In these embodiments, an “inter-phase gap” can be present within a single pulse. The term inter-phase gap where used herein can refer to a period of time between two portions of a pulse comprising a phase change during which zero energy or minimal energy is delivered. The term “quiescent period” where used herein can refer to a period of time during which zero energy or minimal energy is delivered (e.g. insufficient energy to elicit an action potential and/or other neuronal response). The term “inter-pulse gap” where used herein can refer to a quiescent period between the end of one pulse to the onset of the next (sequential) pulse. The terms “pulse train” or “train” where used herein can refer to a series of pulses. The terms “burst”, “burst of pulses” or “burst stimulation” where used herein can refer to a series of pulse trains, each separated by a quiescent period. The term “train-on period” where used herein can refer to a period of time from the beginning of the first pulse to the end of the last pulse of a single train. The term “train-off period” where used herein can refer to a quiescent period between the end of one train and the beginning of the next train. The term “burst-on period” where used herein can refer to a period of time from the beginning of the first pulse of the first train to the end of the last pulse of the last train of a single burst. The term “burst-off period” where used herein can refer to a quiescent period between the end of one burst and the beginning of the next burst. The term “inter-train period” where used herein can refer to a quiescent period between the end of one train and the beginning of the next train. The term “inter-burst period” where used herein can refer to a quiescent period between the end of one burst and the beginning of the next burst. The term “train envelope” where used herein can refer to a curve outlining the amplitude extremes of a series of pulses in a train. The term “burst envelope” where used herein can refer to a curve outlining the amplitude extremes of a series of pulses in a burst. The term “train ramp duration” where used herein can refer to the time from the onset of a train until its train envelope reaches a desired target magnitude. The term “burst ramp duration” where used herein can refer to the time from the onset of a burst until its burst envelope reaches a desired target magnitude.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. For example, it will be appreciated that all features set out in any of the claims (whether independent or dependent) can be combined in any given way.

The present inventive concepts include a medical apparatus and clinical methods for treating a medical condition of a patient, such as a disease or disorder of the patient. Alternatively or additionally, the medical apparatus can be configured for performing a diagnostic and/or prognostic (“diagnostic” herein) procedure on a patient. The patient can comprise a human or other mammalian patient. The medical apparatus can comprise a stimulation or other therapy-providing apparatus. The medical apparatus can comprise an implantable system and an external system. The implantable system can comprise one or more similar and/or dissimilar delivery devices. Each delivery device comprises a housing surrounding one or more therapy-providing components (e.g. stimulation producing components) and/or sensing components. One or more leads (e.g. flexible leads) can be pre-attached to the housing, or the leads can be attachable to the housing (e.g. attached in a clinical procedure in which at least the distal portion of the lead of the delivery device is implanted in a patient).

Each lead can comprise one or more therapy-providing functional elements, such as elements configured to delivery stimulation energy (e.g. electrical, light, and/or sound energy) and/or elements configured to deliver an agent (e.g. a pharmaceutical drug or other agent). Alternatively or additionally, each lead can comprise one or more sensors, such as one or more physiologic sensors. Each lead can comprise one or more electrodes configured to identify the position or change in position of the lead (e.g. position or change in position of the lead as implanted in the patient). In some embodiments, these position-identifying electrodes are further configured as a functional element that provides the therapeutic stimulation energy (e.g. electrical energy).

Each delivery device can comprise one or more antennas configured to receive power and/or data. Each delivery device can comprise a receiver configured to receive the power and/or data from the one or more antennas. Each delivery device can comprise one or more functional elements (e.g. an implantable stimulation element). A functional element of a delivery device can be configured to interface with the patient (e.g. interface with tissue of the patient or interface with any patient location). Alternatively or additionally, a functional element of a delivery device can interface with a portion of its delivery device (e.g. to measure a delivery device parameter). In some embodiments, one or more functional elements of a delivery device can comprise one or more transducers, electrodes, and/or other elements configured to deliver energy to tissue. Alternatively or additionally, one or more functional elements of a delivery device can comprise one or more sensors, such as a sensor configured to record a physiologic parameter of the patient. In some embodiments, one or more functional elements of a delivery device are configured to record device information and/or patient information (e.g. patient physiologic or patient environment information).

Each delivery device can comprise a controller configured to control (e.g. modulate power to, send a signal to, and/or receive a signal from) the one or more functional elements of the delivery device. In some embodiments, a controller of a first delivery device is configured to control one or more other delivery devices (e.g. one or more other delivery devices that have been implanted in the patient). Each delivery device can comprise an energy storage assembly (e.g. a battery and/or a capacitor) configured to provide power to the controller (e.g. a controller comprising a stimulation waveform generator), the receiver and/or the one or more functional elements of the delivery device. In some embodiments, an energy storage assembly is further configured to provide power to an assembly that transmits signals via the antenna of the delivery device (e.g. when the delivery device is further configured to transmit data to one or more other devices of the apparatus). Each delivery device can comprise a housing (e.g. an implantable housing) surrounding the controller and the receiver. In some embodiments, one or more antennas are positioned within the housing of the delivery device. Alternatively or additionally, one or more antennas and/or functional elements can be tethered (e.g. electrically tethered) to the housing of the delivery device. In some embodiments, one or more functional elements are positioned on an implantable lead, such as a flexible lead mechanically fixed or attachable to the delivery device housing and operably connected (e.g. electrically, fluidly, optically and/or mechanically) to one or more components internal to the housing. The implantable lead can be inserted (e.g. tunneled) through tissue of the patient, such that its one or more functional elements are positioned proximate tissue to be treated and/or positioned at an area in which data is to be recorded. In some embodiments, the implantable lead is configured to operably attach to and/or detach from, multiple delivery devices.

The external system of the medical apparatus of the present inventive concepts can comprise one or more similar and/or dissimilar external devices. Each external device can comprise one or more external antennas configured to transmit power and/or data to one or more implanted components of the implantable system. Each external device can comprise an external transmitter configured to drive the one or more external antennas. Each external device can comprise an external power supply configured to provide power to at least the external transmitter. Each external device can comprise an external programmer configured to control the external transmitter and/or an implantable device (e.g. when an external power transmitter is not included in the apparatus or otherwise not present during use). Each external device can comprise an external housing that surrounds at least the external transmitter. In some embodiments, the external housing surrounds the one or more external antennas, the external power supply and/or the external programmer.

The external programmer can comprise a discrete controller separate from the one or more external devices, and/or a controller integrated into one or more external devices. The external programmer can comprise a user interface, such as a user interface configured to set and/or modify one or more treatment and/or data recording settings of the medical apparatus of the present inventive concepts. In some embodiments, the external programmer is configured to collect and/or diagnose recorded patient information, such as to provide the information and/or diagnosis to a clinician of the patient, to a patient family member and/or to the patient themselves. The collected information and/or diagnosis can be used to adjust treatment or other operating parameters of the medical apparatus. In some embodiments, at least two external programmers are included, such as a first external programmer configured for use by the patient, and a second external programmer configured for use by a clinician of the patient.

In some embodiments, a medical apparatus comprises a stimulation apparatus for activating, blocking, affecting or otherwise stimulating (hereinafter “stimulate” or “stimulating”) tissue of a patient, such as nerve tissue or nerve root tissue (hereinafter “nerve”, “nerves”, “nerve tissue” or “nervous system tissue”). The stimulation apparatus comprises an external system configured to transmit power, and an implanted system configured to receive the power from the external system and to deliver therapy (e.g. deliver stimulation energy and/or an agent to tissue). Therapy comprising delivered stimulation energy can comprise one or more stimulation waveforms, such as a stimulation waveform configured to enhance treatment of pain while minimizing undesired effects. The stimulation signal (also referred to as “stimulation energy” herein) delivered by the implanted system can be independent of the power received from the external system, such as to be independent of one or more of: the position of one or more components of the external system; the changing position of one or more components of the external system; the frequency of the power received from the external system; the amplitude of the power received from the external system; changes in amplitude of the power received from the external system; duty cycle of the power received from the external system; envelope of the power received from the external system; and combinations of these.

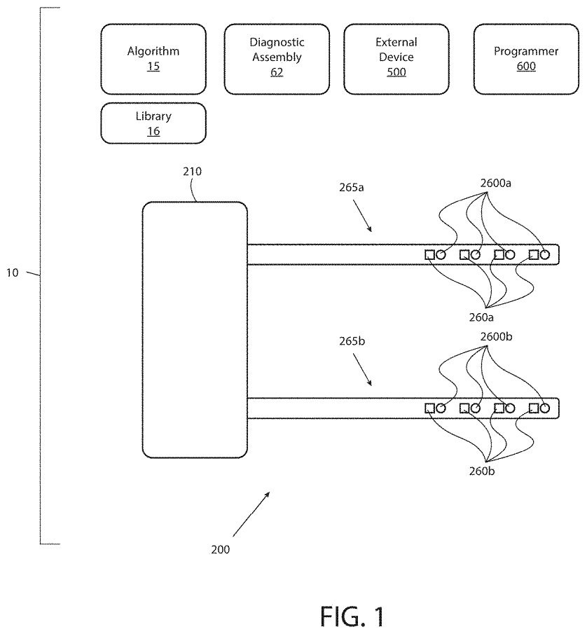

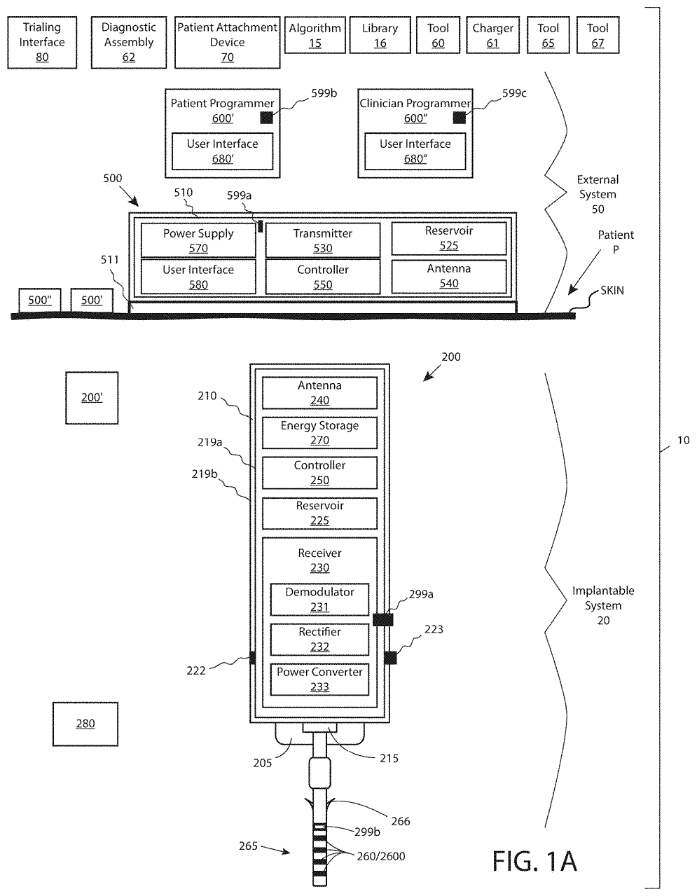

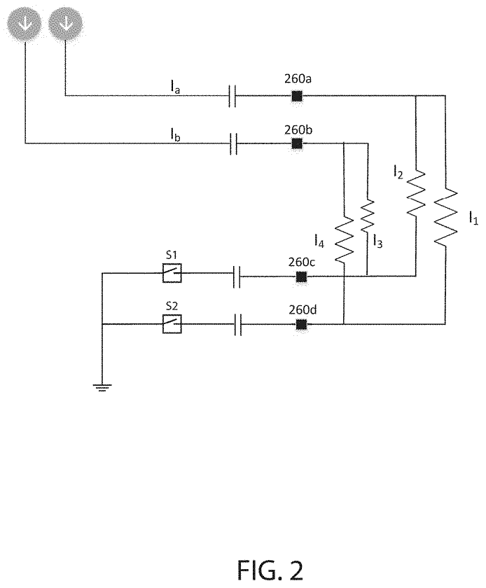

Referring now to FIG. 1, a schematic view of a medical apparatus for a patient is illustrated, consistent with the present inventive concepts. Apparatus 10 comprises delivery device 200, which includes one or more leads, lead 265 (two shown, 265a and 265b, in FIG. 1), each of which extends from a housing, housing 210. Delivery device 200 can be configured to provide a therapy to a patient (e.g. stimulation therapy), and/or to record patient information, such as patient physiologic information. At least a portion (e.g. at least the distal portion) of leads 265 can be configured to be implanted in a patient (i.e. positioned under the skin of the patient). In some embodiments, housing 210 is also configured for implantation in the patient (e.g. when delivery device 200 is implanted in the patient in its entirety). Apparatus 10 can comprise a memory storing instructions for performing one or more algorithms, algorithm 15 shown. The memory may be coupled to a controller 550 of external device 500 of the apparatus 10. Algorithm 15 can be configured to steer electrical current delivered by one or more electrodes 2600 of the one or more leads 265, as described herein. In some embodiments, algorithm 15 comprises one or more mathematical models, such as one or more mathematical models used to measure tissue characteristics and/or otherwise provide information related to the steering of electrical current in tissue (e.g. to stimulate one or more volumes of target tissue). In some embodiments, algorithm 15 is configured to determine stimulation parameters to steer current using a finite element analysis (FEA) model. Alternatively or additionally, algorithm 15 can be configured to steer current using a lumped element model (also referred to as a “lumped parameter model”) such as when tissue is modeled as a set of resistors and capacitors. In some embodiments, algorithm 15 is configured to steer current as described herebelow in reference to FIGS. 2-19.

In some embodiments, apparatus 10 comprises a module for storing data, library 16. Library 16 can include various data regarding one or more components of apparatus 10 (e.g. component functional or other parameter data). Library 16 can include data that is used to steer current in tissue, as described herein. In some embodiments, library 16 includes data comprising models of steering current in various tissue configurations and stimulation element placement geometries (e.g. geometries of stimulation elements 260 described herein), such as models generated using finite element analysis (FEA), as described herein. In some embodiments, algorithm 15 is configured to assess the implantation location and/or implantation geometry of one, two or more leads 265 and “look up” data stored in library 16 in order to determine (e.g. select) a pre-determined stimulation paradigm SP, as described herein.

Each lead 265 can comprise one or more electrodes 2600 (four shown for lead 265a and four shown for lead 265b). In some embodiments, a single lead 265 comprises 1, 2, 3, 4, 6, and/or 8 electrodes 2600. Each electrode 2600 can comprise a component configured to deliver current (also referred to as “source current” herein) and/or receive current (also referred to as “sink current” herein). Each electrode 2600 can be configured to deliver stimulation energy (e.g. in a monopolar and/or bipolar mode). Current transmitted between two or more electrodes 2600 (via tissue in between the two or more electrodes 2600) can be used by apparatus 10 (e.g. used by algorithm 15) to identify (e.g. provide information related to) one or more of the following: the current position of one or more leads 265; the change in position of one or more leads 265 (e.g. the change in position between two or more instances in time); the relative position between two or more leads 265; the change in the relative position between two or more leads 265 (e.g. the change in position between two or more instances in time); and combinations of these, such as is described herein.

In some embodiments, electrodes 2600 comprise electrodes with a length of 0.5 mm, a length of 3.0 mm, or any length in between. Electrodes 2600 can comprise an electrode with an outer diameter of 1.35 mm. Electrodes 2600 can comprise electrodes constructed of platinum and iridium, such as platinum and iridium at a 9:1 ratio. Sets of electrodes 2600 positioned on a single lead 265 can be separated 0.5 mm apart from each other, 4.0 mm apart from each other, or at a separation distance between 0.5 mm and 4.0 mm. Electrodes 2600 can comprise electrodes with one or more coatings and/or finishes, such as a coating or a finish that reduces the impedance of the electrode and/or increases the surface area of the electrode.

In some embodiments, lead 265 comprises a catheter (e.g. a single or multi-lumen catheter) configured to deliver a pharmaceutical drug or other therapeutic agent (e.g. an agent stored within a reservoir of delivery device 200, not shown). Alternatively or additionally, delivery device 200 comprises one or more functional elements configured to provide therapy and/or perform a diagnostic function, such as stimulation elements 260 (four shown for lead 265a and four shown for lead 265b). In some embodiments, one or more stimulation elements 260, and a corresponding electrode 2600, are the same component (i.e. the same electrode), such as when the one or more stimulation elements comprise an electrode configured to deliver stimulation energy in the form of electrical energy. Alternatively or additionally, one or more stimulation elements 260 can comprise a non-electrical energy delivering component (e.g. not an electrode), and an associated electrode 2600 can be positioned adjacent (e.g. attached to) and/or at least proximate the stimulation element 260 (as shown in FIG. 1). For example, one or more stimulation elements 260 can comprise one or more therapy delivery elements, such as: a light energy delivering component (e.g. a lens or a prism configured to deliver laser or other light energy), a sound energy delivering component (e.g. a piezo transducer configured to deliver ultrasound or other sound energy), and/or an agent delivering component (e.g. a needle or an outlet of a lumen), such as when each stimulation element 260 is attached to lead 265 with an electrode 2600 positioned adjacent to (e.g. attached to) and/or at least in close proximity to the stimulation element 260. In some embodiments, one or more stimulation elements 260 can comprise a sensor (e.g. all or a portion of stimulation elements 260 comprise a sensor), and an associated electrode 2600 can be positioned adjacent (e.g. attached to) and/or at least proximate the stimulation element 260, such as when delivery device 200 is configured as a diagnostic device to measure one or more physiologic parameters of a patient.

Apparatus 10 can include one or more devices for transferring power (e.g. via a wired or wireless connection) to delivery device 200, such as external device 500 shown. Alternatively or additionally, external device 500 can be configured to transfer data to, and/or receive data from, delivery device 200 (e.g. via a wired or wireless connection). In some embodiments, external device 500 is configured to be positioned (e.g. via a temporary adhesive and/or strap) above a location in which delivery device 200 has been implanted, and to at least wirelessly transfer power to the implanted delivery device 200.

Apparatus 10 can include a device for programming, delivering data to, and/or otherwise controlling delivery device 200, such as programmer 600 shown (e.g. via control signals sent via a wired or wireless connection).

Apparatus 10 can include one or more devices for gathering information related to the patient and/or the environment of the patient, such as diagnostic assembly 62 shown. Diagnostic assembly 62 can comprise an assembly that is integrated (in whole or in part) into delivery device 200, external device 500, and/or programmer 600.

One or more components of apparatus 10 of FIG. 1 can have similar construction and arrangement to the similar components of apparatus 10 of FIG. 1A described herein. Alternatively or additionally, one or more components of apparatus 10 of FIG. 1 can have similar construction and arrangement to the similar components of the stimulation apparatus described in applicant's co-pending International PCT Patent Application Serial Number PCT/US2020/040766, titled “Stimulation Apparatus”, filed Jul. 2, 2020 [Docket nos. 47476-715.601; NAL-021-PCT].

Referring now to FIG. 1A, a schematic anatomical view of an apparatus for providing a therapy to a patient is illustrated, consistent with the present inventive concepts. Apparatus 10 of FIG. 1A comprises implantable system 20 and external system 50. In some embodiments, apparatus 10 of FIG. 1A, and/or its components, are of similar construction and arrangement to those described in applicant's co-pending International PCT Patent Application Serial Number PCT/US2020/040766, titled “Stimulation Apparatus”, filed Jul. 2, 2020 [Docket nos. 47476-715.601; NAL-021-PCT].

External system 50 transmits transmission signals to one or more components of implantable system 20. These transmission signals can comprise power and/or data. Implantable system 20 comprises one or more devices for delivering a therapy, delivery device 200, shown implanted beneath the skin of patient P. In some embodiments, implantable system 20 comprises multiple similar or dissimilar delivery devices 200 (singly or collectively delivery device 200). Each delivery device 200 can be configured to receive power and data from a transmission signal transmitted by external system 50, such as when stimulation energy delivered to the patient (e.g. to nerve or other tissue of the patient) by delivery device 200 is provided via wireless transmissions signals from external system 50. In some embodiments, implantable system 20 comprises at least two implantable devices, such as delivery device 200 and delivery device 200′ shown in FIG. 1A. Delivery device 200′ can be of similar construction and arrangement to delivery device 200, and/or it can include components of a different configuration. Each delivery device 200 comprises one or more housings, housing 210 shown, which surrounds various other components of device 200 (e.g. a power supply, a receiver, a controller, and/or an antenna, each as described herein). In some embodiments, one or more inner or outer surfaces (or portions of surfaces) of housing 210 includes an insulating and/or shielding layer (e.g. a conductive electromagnetic shielding layer), such as inner coating 219a and/or outer coating 219b shown (singly or collectively coating 219). Coating 219 can comprise an electrically insulating and/or a thermally insulating layer or other coating. In some embodiments, one or more portions of housing 210 comprise an electrically shielding coating, coating 219, while other portions are transmissive to electromagnetic signals such as radiofrequency signals.

Each delivery device 200 comprises one or more stimulation and/or other functional elements, such as stimulation element 260 shown, where stimulation elements 260 are configured to deliver stimulation energy, a stimulating drug or other agent, and/or another form of stimulation (e.g. another form of tissue stimulation) to the patient. Alternatively or additionally, one or more stimulation elements 260 are configured as a sensor (e.g. when comprising an electrode configured to both deliver electrical energy and record electrical signals). Each delivery device 200 can include one or more leads, lead 265 shown, and each lead 265 can include one or more stimulation elements 260. Alternatively or additionally, one or more stimulation elements 260 can be positioned on housing 210 or one or more other components of delivery device 200. In some embodiments, delivery device 200 comprises at least two leads 265, such as is shown in FIG. 1.

Apparatus 10 can comprise a memory storing instructions for performing one or more algorithms, algorithm 15 shown. Algorithm 15 can comprise one or more algorithms configured to determine location information regarding one or more leads 265, as described herein. In some embodiments, algorithm 15 comprises an algorithm that is based on data that is gathered prior to implantation of delivery device 200, such as data gathered during manufacturing of delivery device 200. For example, algorithm 15 can be based on electrode 2600 impedance data that was recorded prior to implantation of lead 265, such as when electrode 2600 comprises electrodes with a coating and/or an enhanced surface (e.g. resulting in a lowered impedance). In some embodiments, algorithm 15 comprises one or more mathematical models, such as one or more mathematical models used to measure tissue characteristics and/or otherwise provide information related to the steering of current in tissue. In some embodiments, algorithm 15 is configured to steer current delivered by stimulation elements 260, such as is described herebelow in reference to FIGS. 2-19. Apparatus 10 can further include library 16 described in reference to FIG. 1 and otherwise herein, such as a library of data used to steer current delivered by stimulation elements 260. In some embodiments, library 16 includes data comprising models of steering current in various tissue configurations and stimulation element 260 placement geometries, such as models generated using lumped element analysis, and/or finite element analysis (FEA), as described herein.

Each lead 265 can comprise one or more electrodes 2600 (four shown for lead 265 in FIG. 1A). In some embodiments, a single lead 265 comprises a set of at least 1, 2, 3, 4, 6, and/or 8 electrodes 2600. Each electrode 2600 can comprise a component configured to deliver current (also referred to as “source current” herein) and/or receive current (also referred to as “sink current” herein). In some embodiments, housing 210 comprises at least a portion that is conductive and configured as an electrode (e.g. configured to source and/or sink current as described herein). Current transmitted between two or more electrodes 2600 (via tissue in between the two or more electrodes 2600) can be used by apparatus 10 (e.g. used by algorithm 15) to identify (e.g. provide information related to) one or more of the following: the current position of one or more leads 265; the change in position of one or more leads 265; the relative position between two or more leads 265; the change in the relative position between two or more leads 265; and combinations of these, such as is described herein. In some embodiments, algorithm 15 is configured to provide lead 265 location information, such as is described in applicant's co-pending International PCT Patent Application Serial Number PCT/US2020/066901, titled “Systems with Implanted Conduit Tracking”, filed Dec. 23, 2020 [Docket nos. 47476-716.601; NAL-022-PCT]. In some embodiments, one or more electrodes 2600 comprise the same component as an associated set of one or more stimulation elements 260, such as the four electrodes 2600 that comprise the same four elements 260 of FIG. 1A.

Each delivery device 200 can comprise one or more other types of functional elements, such as functional element 299a shown positioned proximate housing 210 (e.g. within and/or on the external surface of housing 210) and/or functional element 299b shown positioned on lead 265. Functional element 299a and/or 299b (singly or collectively functional element 299) can comprise a transducer, a sensor, and/or other functional element as described herein. In some embodiments, a functional element 299 comprises a visualizable marker, such as a radiopaque marker, an ultrasonically visible marker, and/or a magnetic marker.

External system 50 can comprise an external device 500, which includes one or more housings, housing 510 shown, which surrounds various other components of device 500. In some embodiments, external system 50 comprises multiple external devices 500 (singly or collectively external device 500), such as an external device as is described in applicant's co-pending U.S. patent application Ser. No. 16/104,829, titled “Apparatus with Enhanced Stimulation Waveforms”, filed Aug. 17, 2018 [Docket nos. 47476-708.301; NAL-014-US]. In some embodiments, external system 50 comprises at least two, or at least three external devices (e.g. at least two external devices configured to deliver power and/or data to one or more delivery devices 200), such as external device 500, external device 500′, and external device 500″ shown in FIG. 1A. External device 500′ and/or 500″ can be of similar construction and arrangement to external device 500, and these devices can include components of a different configuration.

External system 50 can comprise one or more programming devices, programmer 600, such as patient programmer 600′ and/or clinician programmer 600″ shown. Patient programmer 600′ and clinician programmer 600″ (singly or collectively programmer 600) each comprise a user interface, such as user interfaces 680′ and 680″ shown (singly or collectively user interface 680). Programmer 600 can be configured to control one or more external devices 500. Alternatively or additionally, programmer 600 can be configured to control one or more delivery devices 200 (e.g. when no external device 500 is included in apparatus 10 or at least no external device 500 is available to communicate with a delivery device 200). Patient programmer 600′ can be configured to be used by the patient, patient caregiver (e.g. clinician of the patient), and/or a family member of the patient. In some embodiments, one or more external devices 500 comprise all or a portion of a programmer 600, such as when all or a portion of user interface 680 is integrated into housing 510 of external device 500. In some embodiments, apparatus 10 comprises multiple programmers 600, such as one or more patient programmers 600′ and/or one or more clinician programmers 600″.

Clinician programmer 600″ can be of similar construction and arrangement to patient programmer 600′. In some embodiments, clinician programmer 600″ provides additional functions not available in patient programmer 600′. In some embodiments, clinician programmer 600″ can modify the programming of patient programmer 600′ (e.g. modify the programming options available to the patient or family member of the patient).

External system 50 can comprise one, two, three, or more functional elements, such as functional elements 599a, 599b, and/or 599c (singly or collectively functional element 599), shown positioned in external device 500, patient programmer 600′, and clinician programmer 600″, respectively. Each functional element 599 can comprise a functional element as defined hereabove (e.g. a sensor, a transducer, and/or other functional element as described herein). In some embodiments, a functional element 599 comprises a needle, a catheter (e.g. a distal portion of a catheter), an iontophoretic element or a porous membrane, such as an agent delivery element configured to deliver one or more agents contained (e.g. one or more agents in a reservoir, such as reservoir 525 shown) within an external device 500 and delivered into the patient (e.g. into subcutaneous tissue, into muscle tissue and/or into a blood vessel such as a vein).

As described hereabove, external system 50 can be configured to transmit power and/or data (e.g. implantable system 20 configuration data) to one or more delivery devices 200 of implantable system 20. Implantable system 20 configuration data provided by external system 50 (e.g. via one or more antennas, antenna 540 shown, of one or more external devices 500) can include when to initiate stimulation delivery (e.g. energy delivery), and/or when to stop stimulation delivery, and/or it can include data related to the value or change to a value of one or more stimulation variables as described hereabove. The configuration data can include a stimulation parameter such as an agent (e.g. a pharmaceutical agent) delivery stimulation parameter selected from the group consisting of: initiation of agent delivery; cessation of agent delivery; amount of agent to be delivered; volume of agent to be delivered; rate of agent delivery; duration of agent delivery; time of agent delivery initiation; and combinations of these. The configuration data can include a sensing parameter, such as a sensing parameter selected from the group consisting of: initiation of sensor recording; cessation of sensor recording; frequency of sensor recording; resolution of sensor recording; thresholds of sensor recording; sampling frequency of sensor recording; dynamic range of sensor recording; initiation of calibration of sensor recording; and combinations of these.

As described herein, external system 50 can comprise one or more external devices 500. External system 50 can comprise one or more antennas 540, such as when a single external device 500 comprises one or more antennas 540, and/or when multiple external devices 500 each comprise one or more antennas 540. The one or more antennas 540 can transmit power and/or data to one or more antennas, antennas 240, of implantable system 20, such as when a single delivery device 200 comprises one or more antennas 240, and/or when multiple delivery devices 200 each comprise one or more antennas 240.

In some embodiments, one or more external devices 500 are configured to transmit both power and data (e.g. simultaneously and/or sequentially) to one or more delivery devices 200. In some embodiments, one or more external devices 500 are further configured to receive data from one or more delivery devices 200 (e.g. via data transmitted by one or more antennas 240 of one or more delivery devices 200). Each external device 500 can comprise housing 510, a power supply 570, a transmitter 530, a controller 550, and/or one or more antennas 540, each shown in FIG. 1A and described herein. Each external device 500 can further comprise one or more functional elements 599a, such as a functional element comprising a sensor, electrode, energy delivery element, a magnetic field-generating transducer, and/or any transducer, also described in detail herebelow. In some embodiments, a functional element 599a comprises one or more sensors configured to monitor performance of external device 500 (e.g. to monitor voltage of power supply 570, quality of transmission of power and/or data to implantable system 20, temperature of a portion of an external device 500, and the like).

External system 50 can transmit power and/or data with a transmission signal comprising at least one wavelength, 2. External system 50 and/or implantable system 20 can be configured such that the distance between an external antenna 540 transmitting the power and/or data and one or more implantable antennas 240 receiving the power and/or data transmission signal is equal to between 0.1λ and 10.0λ, such as between 0.2λ and 2.0λ. In some embodiments, one or more transmission signals are delivered by a transmitter, transmitter 530, at a frequency range between 10 MHz and 10.6 GHz, such as between 0.1 GHz and 10.6 GHz, between 10 MHz and 3.0 GHz, between 40 MHz and 1.5 GHz, between 10 MHz and 100 MHz, between 0.902 GHz and 0.928 GHz, in a frequency range proximate to 40.68 MHz, in a frequency range proximate to 866 MHz, or approximately between 863 MHz and 870 MHz. Transmitter 530 can comprise a transmitter that produces a transmission signal with a power level between 0.01 W and 4.0 W, such as a transmission signal with a power level between 0.01 W and 2.0 W or between 0.2 W and 1.0 W.

Housing 510 can comprise an adhesive element, spacer 511 shown, which can be configured as an adhesive element that temporarily attaches an external device 500 to the patient's skin. Alternatively or additionally, housing 510 can be constructed and arranged to engage (e.g. fit in the pocket of) a patient attachment device, such as patient attachment device 70 described herein (e.g. a clip that is adhesively attached to the patient's skin).

In some embodiments, transmitter 530 (and/or another component of external system 50) is further configured as a receiver (e.g. can further include a receiver, in addition to a transmitter or include a transmitter that further functions as a receiver), such as to receive data from implantable system 20. For example, a transmitter 530 can be configured to receive data via one or more antennas 240 of one or more delivery devices 200. Data received can include patient information (e.g. patient physiologic information, patient environment information or other patient information) and/or information related to an implantable system 20 parameter (e.g. a delivery device 200 stimulation parameter and/or another configuration parameter as described herein).

Each power supply 570 (singly or collectively power supply 570) can be operably attached to a transmitter 530, and one or more other electronic components of each external device 500. Power supply 570 can comprise a power supplying and/or energy storage element selected from the group consisting of: battery; replaceable battery (e.g. via a battery door of housing 510); rechargeable battery; AC power converter; capacitor; and combinations of these. In some embodiments, power supply 570 is configured to provide a voltage of at least 3V. In some embodiments, power supply 570 is configured to provide a capacity between 1 Watt-hour and 75 Watt-hours, such as a battery or capacitor with a capacity of approximately 5 Watt-hours. In some embodiments, power supply 570 comprises an AC power source. Power supply 570 can include voltage and/or current control circuitry. Alternatively or additionally, power supply 570 can include charging circuitry, such as circuitry configured to interface a rechargeable battery with an external charging device.

Each external device 500 can include one or more user interface components, user interface 580 shown, such as to allow the patient, user and/or other operator of apparatus 10 to adjust one or more parameters of apparatus 10. User interface 580 can include one or more user input components (e.g. buttons, slides, knobs, and the like) and/or one or more user output components (e.g. lights, displays and the like). In some embodiments, user interface 580 includes one or more controls configured to provide a water-ingress-resistant barrier.

In some embodiments, housing 210 comprises an array of feedthroughs, not shown. In some embodiments, housing 210 is surrounded (e.g. partially or fully surrounded) by a covering, such as a flexible and/or non-conductive covering, such as a covering made of an elastomer.

Each delivery device 200 can include one or more energy storage assemblies 270 (singly or collectively energy storage assembly 270). Each assembly 270 can comprise one or more implantable energy storage components, such as one or more batteries (e.g. rechargeable batteries) and/or capacitors (e.g. a supercapacitor). Energy storage assembly 270 can be configured to provide power to one or more of: one or more stimulation elements 260; controller 250; receiver 230; and combinations of these. In some embodiments, energy storage assembly 270 further provides power to one or more antennas 240 and/or circuitry configured to transmit data via antenna 240. In some embodiments, energy storage assembly 270 includes digital control for charge/discharge rates, voltage outputs, current outputs, and/or system power distribution and/or management.

Energy storage assembly 270 can comprise one or more capacitors with a single or collective capacitance between 0.01 μF and 10 F, such as a capacitance between 1 μF and 1.0 mF, or between 1 μF and 10 μF. The energy storage assembly 270 can comprise one or more capacitors with capacitance between 1 mF and 10 F, such as when energy storage assembly 270 comprises a super-capacitor and/or an ultra-capacitor. Such large capacitance can be used to store sufficient charge to maintain operation (e.g. maintain delivery of stimulation energy and/or delivery of an agent) without the use (e.g. sufficient proximity) of an associated external device 500. A capacitor or other energy storage element (e.g. a battery) can be chosen to provide sufficient energy to maintain operation for at least 30 seconds, at least 2 minutes, at least 5 minutes, at least 30 minutes, and up to several hours or more (e.g. during showering, swimming or other physical activity). In some embodiments, energy storage assembly 270 is configured to provide continuous and/or intermittent stimulation energy for at least one charge-balanced pulse (e.g. for the duration of at least one charge-balanced pulse). In some embodiments, a capacitor, battery or other energy storage element is configured to provide stimulation energy without receiving externally supplied power for periods of at least 1 hour, at least 1 day, at least 1 month or at least 1 year. Energy storage assembly 270 can comprise one or more capacitors with a breakdown voltage above 1.0V, such as a breakdown voltage above 1.5V, 4.0V, 10V, or 15V. In some embodiments, energy storage assembly 270 can comprise capacitors distributed outside of housing 210, such as when one or more capacitors are distributed along lead 265. Energy storage assembly 270 can comprise one or more capacitors with low self-leakage, such as to maintain stored energy for longer periods of time.

In some embodiments, during use (e.g. during a period of providing stimulation or other function) delivery device 200 receives power regularly from external system 50 (e.g. relatively continuously while delivery device 200 delivers stimulation energy), and energy storage assembly 270 comprises a relatively small battery or capacitor, such as a battery or capacitor that has an energy storage capacity of less than or equal to 0.6 Joules, 7 Joules or 40 Joules.

Delivery device 200 can include one or more controllers 250 (singly or collectively controller 250), which can be configured to control one or more stimulation elements 260, such as a stimulation element 260 comprising a stimulation-based transducer (e.g. an electrode or other energy delivery element) and/or a sensor (e.g. a physiologic sensor and/or a sensor configured to monitor a delivery device 200 parameter). In some embodiments, controller 250 is configured to transmit a stimulation signal (e.g. transmit stimulation energy configured in one or more stimulation waveforms) to one or more stimulation elements 260 (e.g. one or more stimulation elements 260 comprising an electrode and/or other energy delivery element), independent of the power signal received by one or more antennas 240 (e.g. independent of power transmitted by external system 50), such as by using energy stored in energy storage assembly 270. In these embodiments, the power signal and/or the RF path for the power signal can be adjusted to optimize power efficiency (e.g. by tuning matching network on transmitter 530 and/or receiver 230; configuring antennas 540 and/or 240 in an array; tuning operating frequency; duty cycling the power signal; adjusting antenna 540 and/or 240 position; and the like), and a stimulation signal can be precisely delivered (e.g. by using energy stored on energy storage assembly 270 and generating stimulation signal locally on the delivery device 200) to ensure clinical efficacy. Also, if the power signal transmission (also referred to as “power link”) is perturbed unexpectedly, the stimulation signal can be configured so that it is not significantly affected (e.g. unaffected). In some configurations, the stimulation signal being delivered by one or more delivery devices 200 is insensitive to interference that may be present. In these embodiments, a power transmission signal and stimulation signal can vary in one or more of: amplitude; changes in amplitude; average amplitude; frequency; changes in frequency; average frequency; phase; changes in phase; average phase; waveform shape; pulse shape; duty cycle; polarity; and combinations of these.

Controller 250 can receive commands from a receiver, receiver 230, such as one or more commands related to one or more delivery device 200 configuration parameters selected from the group consisting of: stimulation parameter; data rate of receiver; data rate of data transmitted by the first delivery device 200 at least one implantable antenna 240; stimulation element 260 configuration; state of controller 250; antenna 240 impedance; clock frequency; sensor configuration; electrode configuration; power management parameter; energy storage assembly parameter; agent delivery parameter; sensor configuration parameter; and combinations of these.

Controller 250 and/or any other component of each delivery device 200 can comprise an integrated circuit comprising one or more components selected from the group consisting of: matching network; rectifier; DC-DC converter; regulator; bandgap reference; overvoltage protection; overcurrent protection; active charge balance circuit; analog to digital converter (ADC); digital to analog converter (DAC); current driver; voltage driver; digital controller; clock generator; data receiver; data demodulator; data modulator; data transmitter; electrode drivers; sensing interface analog front end; power management circuit; energy storage interface; memory register; timing circuit; and combinations of these.

One or more receivers 230 (singly or collectively receiver 230) can comprise one or more components, such as demodulator 231, rectifier 232, and/or power converter 233 shown in FIG. 1A. In some embodiments, receiver 230 can comprise a DC-DC converter such as a boost converter. Receiver 230 can comprise a data receiver, such as a data receiver including an envelope detector and demodulator and/or an envelope averaging circuit. In some embodiments, one or more antennas 240 separately connect to one or more receivers 230. In some embodiments, one or more antennas 240 connect to a single receiver 230, such as via a series connection or a parallel connection.

One or more delivery devices 200 can be configured to transmit a data signal to external system 50. In some embodiments, receiver 230 is configured to drive one or more antennas 240 to transmit data to external system 50 (e.g. receiver 230 is further configured as a transmitter that wirelessly transmits data to an antenna 540 of an external device 500). Alternatively or additionally, delivery device 200 can be configured to transmit a data signal by having receiver 230 adjust a load impedance to backscatter energy, such as a backscattering of energy which can be detected by external system 50. In some embodiments, data transmission is accomplished by receiver 230 manipulating a signal at a tissue interface, such as to transmit a data signal using body conduction.

Demodulator 231 can comprise circuitry that asynchronously recovers signals modulated on the power signal provided by external system 50, and that converts the modulated signals into digital signals. In some embodiments, demodulator 231 asynchronously recovers the modulated signal by comparing a dynamically generated moving average with the envelope, outputting a high voltage when the envelope is greater than the moving average and a low voltage when the envelope is less than the moving average. Data can then be extracted from this resulting digital signal from the width and/or amplitude of the pulses in the signal, according to the encoding method used by external system 50. In some embodiments, demodulator 231 recovers a digital signal that is used as timing information for a delivery device 200, similar to an on-chip clock. The recovered clock signal can also be used to synchronize an on-chip clock generator of controller 250, such as through the use of a frequency and/or phase locked loop (FLL or PLL).

Rectifier 232 can comprise a power signal rectifier, such as to provide power to the energy storage assembly 270 and/or controller 250. In some embodiments, rectifier 232 comprises one or more self-driven synchronous rectifier (SDSR) stages connected in charge-pump configuration, to boost the voltage from input RF amplitude to the rectifier to a higher voltage. The boosted voltage can directly charge energy storage assembly 270, or it can be further boosted by a DC-DC converter or boost converter. In some embodiments, rectifier 232 comprises diode-capacitor ladder stages instead of, or in addition to, SDSR stages. On-chip diodes, such as Schottky diodes, or off-chip diodes can be used in one or more rectifier 232 stages. For maximum efficiency, the rectification elements, such as diodes, can be optimized to minimize forward conduction and/or reverse conduction losses by properly sizing the components and selecting appropriate number of stages based on the input RF voltage and load current.

Power converter 233 can comprise one or more voltage conversion elements such as DC-DC converters that boost or otherwise change the voltage to a desired level. In some embodiments, voltage conversion is achieved with a buck-boost converter, a boost converter, a switched capacitor, and/or charge pumps. One or more power converters 233 can interface with energy storage assembly 270 and charge up associated energy storage components to desired voltages. In some embodiments, power converter 233 receives control signals from controller 250, such as to configure voltages, currents, charge/discharge rates, switching frequencies, and/or other operating parameters of power converter 233.

In some embodiments, delivery device 200 comprises one or more antennas 240 positioned on a substrate, such as a printed circuit board (PCB), a flexible printed circuit board and/or a foldable substrate (e.g. a substrate comprising rigid portions and hinged portions). In some embodiments, the substrate is folded or otherwise pivoted to position the various antennas 240 on differently oriented planes, such as multiple planes oriented between 5° and 90° relative to each other, such as two antennas 240 positioned on two planes oriented between 30° and 90° or between 40° and 90° relative to each other, or three antennas 240 positioned on three planes oriented between 5° and 60° relative to each other. Two or more antennas 240 can be positioned on two or more different planes that are approximately 45° relative to each other, or approximately 60° or approximately 90° relative to each other.

One or more antennas 240 can comprise an antenna selected from the group consisting of: loop antenna; multiple-turn loop antenna; planar loop antenna; coil antenna; dipole antenna; electric dipole antenna; magnetic dipole antenna; patch antenna; loaded dipole antenna; concentric loop antenna; loop antenna with ferrite core; and combinations of these. One or more antennas 240 can comprise a loop antenna, such as an elongated loop antenna or a multiple-turn loop antenna.

One or more antennas 240 can comprise a minor axis and a major axis. In some embodiments, one or more antennas 240 comprise a minor axis between 1 mm and 8 mm, such as between 2 mm and 5 mm. In some embodiments, one or more antennas 240 comprise a major axis between 3 mm and 15 mm, such as between 4 mm and 8 mm. In some embodiments, one or more antennas 240 comprise a major axis above 3 mm, such as between 3 mm and 15 mm, such as when the antenna 240 is positioned outside of housing 210.

One or more antennas 240 can be positioned inside of housing 210. Alternatively or additionally, one or more antennas 240 can be positioned outside of housing 210. Implantable system 20, one or more delivery devices 200 and/or one or more antennas 240 can be configured to be positioned at a desired depth beneath the patient's skin, such as at a depth between 0.5 cm and 7.0 cm, such as a depth of between 1.0 cm and 3.0 cm.

One or more implantable leads 265 (singly or collectively lead 265) can be attached to one or more housings 210, such as a lead 265 comprising one or more stimulation elements 260. Lead 265 can comprise one or more stimulation elements 260 configured as a stimulation element (e.g. an electrode configured to deliver electrical energy in monopolar or bipolar mode or an agent delivery element such as an output port fluidly connected to a reservoir within housing 210). Alternatively or additionally, lead 265 can comprise one or more stimulation elements 260 and/or functional elements 299b that is configured as a physiologic sensor (e.g. an electrode configured to record electrical activity of tissue or another physiologic sensor as described herein). Alternatively or additionally, lead 265 can comprise one or more stimulation elements 260 and/or functional elements 299b that is configured to transmit signals through tissue to external system 50, such as through body conduction.

In some embodiments, delivery device 200 comprises a connector, connector 215, that operably attaches (e.g. electrically attaches) one or more stimulation elements 260 to one or more components (e.g. electronic components) internal to housing 210 (e.g. to transfer power and/or data therebetween). In some embodiments, connector 215 is operably attached (e.g. in a manufacturing process) or attachable (e.g. in a clinical procedure) to lead 265 as shown in FIG. 1A. Alternatively, connector 215 can be operably attached and/or attachable to a lead connection assembly, assembly 280, which in turn can be attached to a lead 265. In some embodiments, connector 215 passes through an opening in housing 210, in a feed-through arrangement. In some embodiments, an overmold or other sealing element, sealing element 205 shown, provides a seal about connector 215, the opening in housing 210 and/or the interface between connector 215 and housing 210.

In some embodiments, lead 265 comprises a diameter between 1 mm and 4 mm, such as a diameter between 1 mm and 2 mm, such as a lead with a diameter of approximately 1.35 mm. In some embodiments, lead 265 comprises a length between 3 cm and 60 cm, such as a length between 6 cm and 30 cm. One or more leads 265 can include between 2 and 64 stimulation elements 260, such as when a lead 265 comprises between 2 and 64 electrodes, such as between 4 and 32 electrodes. In some embodiments, lead 265 comprises a paddle lead. In some embodiments, lead 265 comprises a single or multi-lumen catheter, such as when an attached delivery device 200 is configured as an agent delivery apparatus as described herein (e.g. a stimulation element 260 configured as a catheter comprises at least a portion of lead 265).

In some embodiments, lead 265 comprises one or more tines, such as tines 266 shown. Tines 266 can be configured to anchor or otherwise stabilize (“anchor” or “stabilize” herein) lead 265 relative to patient tissue, such as to prevent undesired movement during and/or after an implantation procedure for lead 265. One or more tines 266 can be configured to biodegrade after implantation in the patient, such that the stabilization provided is temporary. Tines 266 can be configured to biodegrade over a time period of approximately 4 to 12 weeks. In some embodiments, biodegradable tines 266 are configured to be incorporated when lead 265 stimulation elements 260 are positioned to stimulate a peripheral nerve (e.g. lead 265 is implanted such that one or more stimulation elements 260 are positioned proximate one or more peripheral nerves).

As described herein, one or more leads 265 can be positioned to stimulate the spinal cord, such as via percutaneous insertion of a lead 265 in the epidural space or surgical implantation of the lead 265 (e.g. a paddle lead) in the epidural space. A lead 265 can be placed such that one or more stimulation elements 260 (e.g. one or more electrodes) are positioned from T5-S5, such as to capture the area of pain or reduced circulation of the leg or foot. One or more stimulation elements 260 of one or more leads 265 can be positioned from C2 to T8, such as to capture the area of pain or reduced circulation of the arm or hand. One or more leads 265 can be placed along the midline, unilaterally and/or bilaterally over the dorsal columns, in the gutter (over dorsal roots) and/or in the dorsal root entry zone. Leads 265 can span several vertebral levels or they can be positioned to span a single level.

One or more stimulation elements 260 (singly or collectively stimulation element 260) and/or functional element 299 (e.g. functional element 299a and/or 299b) can comprise one or more sensors, transducers and/or other functional elements. In some embodiments, one or more stimulation elements 260 and/or functional elements 299 comprise at least one sensor and/or at least one transducer (e.g. a single stimulation element 260 or multiple stimulation elements 260). In some embodiments, stimulation element 260 and/or functional element 299 comprises a functional element configured to provide a therapy, such as one or more stimulation elements 260 configured to deliver an agent to tissue (e.g. a needle or catheter), to deliver energy to tissue and/or to otherwise therapeutically affect tissue. In some embodiments, stimulation element 260 and/or functional element 299 comprises one or more functional elements configured to record patient information, such as when stimulation element 260 and/or functional element 299 comprises one or more sensors configured to measure a patient physiologic parameter, as described herein. In some embodiments, stimulation element 260 and/or functional element 299 comprises one or more sensors configured to record a delivery device 200 parameter, also as described herein.

One or more stimulation elements 260 can be positioned on lead 265 as shown in FIG. 1A. Alternatively or additionally, one or more stimulation elements 260 can be positioned on housing 210. One or more functional elements 299 can be positioned on lead 265 (e.g. functional element 299b shown) and/or positioned on and/or within housing 210 (e.g. functional element 299a shown).

Stimulation element 260 can comprise one or more stimulation elements positioned at one or more internal body locations. Stimulation element 260 can comprise one or more stimulation elements positioned to interface with (e.g. deliver energy to and/or record a physiologic parameter from) spinal cord tissue, spinal canal tissue, epidural space tissue, spinal root tissue (dorsal or ventral), dorsal root ganglion, nerve tissue (e.g. peripheral nerve tissue, spinal nerve tissue or cranial nerve tissue), brain tissue, ganglia (e.g. sympathetic or parasympathetic) and/or a plexus. In some embodiments, stimulation element 260 comprises one or more elements positioned proximate and/or within one or more tissue types and/or locations selected from the group consisting of: one or more nerves; one or more locations along, in and/or proximate to the spinal cord; peripheral nerves of the spinal cord including locations around the back; the knee; the tibial nerve (and/or sensory fibers that lead to the tibial nerve); the occipital nerve; the sphenopalatine ganglion; the sacral and/or pudendal nerve; brain tissue, such as the thalamus; baroreceptors in a blood vessel wall, such as in the carotid artery; one or more muscles; the medial nerve; the hypoglossal nerve and/or one or more muscles of the tongue; cardiac tissue; the anal sphincter; the dorsal root ganglion; motor nerves; muscle tissue; the spine; the vagus nerve; the renal nerve; an organ; the heart; the liver; the kidney; an artery; a vein; bone; and combinations of these, such as to stimulate and/or record data from the tissue and/or location in which the stimulation element 260 is positioned proximate to and/or within. In some embodiments, apparatus 10, delivery device 200 and/or stimulation element 260 are configured to stimulate spinal nerves, peripheral nerves and/or other tissue as described in applicant's co-pending U.S. patent application Ser. No. 16/993,999, titled “Apparatus for Peripheral or Spinal Stimulation”, filed Aug. 14, 2020 [Docket nos. 47476-707.302; NAL-012-US-CON1].

In some embodiments, stimulation element 260 and/or functional element 299 comprises one or more sensors configured to record data representing a parameter of delivery device 200. In these embodiments, stimulation element 260 and/or functional element 299 can comprise one or more sensors selected from the group consisting of: an energy sensor; a voltage sensor; a current sensor; a temperature sensor (e.g. a temperature of one or more components of delivery device 200); a contamination detector (e.g. to detect undesired material that has passed through housing 210); an antenna matching and/or mismatching assessment sensor; power transfer sensor; link gain sensor; power use sensor; energy level sensor; energy charge rate sensor; energy discharge rate sensor; impedance sensor; load impedance sensor; instantaneous power usage sensor; average power usage sensor; bit error rate sensor; signal integrity sensor; and combinations of these. Apparatus 10 can be configured to analyze (e.g. via implantable controller 250, programmer 600 and/or diagnostic assembly 62 described herein) the data recorded by stimulation element 260 and/or functional element 299 to assess one or more of: power transfer; link gain; power use; energy within energy storage assembly 270; performance of energy storage assembly 270; expected life of energy storage assembly 270; discharge rate of energy storage assembly 270; ripple or other variations of energy storage assembly 270; matching of antenna 240 and 540; communication error rate between delivery device 200 and external device 500; integrity of transmission between delivery device 200 and external device 500; and combinations of these. A stimulation element 260 can be configured to record temperature, such as when apparatus 10 is configured to deactivate or otherwise modify the performance of a delivery device 200 when the recorded temperature exceeds a threshold.

In some embodiments, one or more stimulation elements 260 comprise a transducer configured to deliver energy to tissue, such as to treat pain and/or to otherwise stimulate or affect tissue. In some embodiments, stimulation element 260 comprises a stimulation element, such as one or more transducers selected from the group consisting of: an electrode; an energy delivery element such as an electrical energy delivery element, a light energy delivery element, a laser light energy delivery element, a sound energy delivery element, a subsonic sound energy delivery element and/or an ultrasonic sound delivery element; an electromagnetic field generating element; a magnetic field generating element; a mechanical transducer (e.g. delivering mechanical energy to tissue); a tissue manipulating element; a heat generating element; a cooling (e.g. cryogenic or otherwise heat extracting energy) element; an agent delivery element such as a pharmaceutical drug delivery element; and combinations of these. In some embodiments, one or more stimulation elements 260 comprise one or more electrodes configured to deliver energy to tissue and/or to sense a patient parameter (e.g. electrical activity of tissue or other patient physiologic parameter). In these embodiments, one or more stimulation elements 260 can comprise one or more electrodes selected from the group consisting of: microelectrode; cuff electrode; array of electrodes; linear array of electrodes; circular array of electrodes; paddle-shaped array of electrodes; bifurcated electrodes; and combinations of these.

In some embodiments, one or more stimulation elements 260 comprises a drug or other agent delivery element, such as a needle, port, iontophoretic element, catheter, or other agent delivering element that is connected to a reservoir of agent positioned within housing 210 (e.g. reservoir 225 shown). In some embodiments, one or more stimulation elements 260 comprise a drug eluting element configured to improve biocompatibility of implantable system 20.

In some embodiments, apparatus 10 (e.g. via stimulation element 260, functional element 299, and/or functional element 599) is configured to both record one or more patient parameters, and also to perform a medical therapy (e.g. stimulation of tissue with energy and/or an agent). In these embodiments, the medical therapy can be performed in a closed-loop fashion, such as when energy and/or agent delivery is modified based on the measured one or more patient physiologic parameters.

One or more portions of delivery device 200 or other component of implantable system 20 can be configured to be visualized or contain a visualizable portion or other visualizable element, such as visualizable element 222 shown. Visualizable element 222 can comprise a material selected from the group consisting of: radiopaque material; ultrasonically reflective material; magnetic material; and combinations of these. In these embodiments, each delivery device 200 can be visualized (e.g. during and/or after implantation) via an imaging device such as a CT, X-ray, fluoroscope, ultrasound imager and/or MRI.

In some embodiments, delivery device 200 and/or another component of apparatus 10 can include one or more features to prevent or at least reduce migration of device 200 within the patient's body. In some embodiments, one or more delivery devices 200 comprises one or more anchor elements configured to secure one or more portions of delivery device 200 to tissue, such as anchor element 223 shown. Anchor element 223 can comprise one or more anchoring elements selected from the group consisting of: a sleeve such as a silicone sleeve; suture tab; suture eyelet; bone anchor; wire loops; porous mesh; penetrable wing; penetrable tab; bone screw eyelet; tine; pincers; suture slits; and combinations of these. While anchor element 223 is shown proximate housing 210 (e.g. to fixedly attach housing 210 to tissue), in some embodiments anchor element 223 surrounds or is otherwise proximate lead 265 (e.g. to fixedly attach lead 265 to tissue). In some embodiments, anchor element 223 comprises a porous mesh that surrounds all or a portion of housing 210. The porous mesh can be configured to promote tissue ingrowth, such as to prevent or at least limit (“prevent” herein) migration of housing 210 when delivery device 200 is implanted in the patient. In some embodiments, anchor element 223 comprises a mesh that is attached to the top side of delivery device 200 (side in closest proximity to the patient's skin), such as to prevent housing 210 from migrating away from the patient's skin (e.g. prevent from migrating deeper into the patient).

In some embodiments, apparatus 10 comprises one or more tools, tool 60 shown. Tool 60 can comprise a data logging and/or analysis tool configured to receive data from external system 50 or implantable system 20, such as data comprising: diagnostic information recorded by external system 50 and/or implantable system 20; therapeutic information recorded by external system 50 and/or implantable system 20; patient information (e.g. patient physiologic information) recorded by implantable system 20; patient environment information recorded by implantable system 20; and combinations of these. Tool 60 can be configured to receive data from wired or wireless (e.g. Bluetooth) means. Tool 60 can comprise a tool selected from the group consisting of: a data logging and/or storage tool; a data analysis tool; a network such as a LAN or the Internet; a cell phone; and combinations of these.

Apparatus 10 can include a battery charging assembly, charger 61 shown, such as an assembly configured to recharge one or more power supplies 570 and/or other component of apparatus 10 comprising a rechargeable battery or capacitor.

Apparatus 10 can include one or more implantation tools, tool 65 shown. Implantation tool 65 can comprise an introducer, tunneller, and/or other implantation tool constructed and arranged to aid in the implantation of housing 210, implantable antenna 240, lead 265 and/or one or more stimulation elements 260. In some embodiments, tool 65 comprises a component configured to anchor delivery device 200 to tissue, such as a mesh or wrap that slides around at least a portion of delivery device 200 and is configured to engage tissue (e.g. via tissue ingrowth) or be engaged with tissue (e.g. via suture or clips).

Apparatus 10 can include one or more placement tools, positioning tool 67 shown, which can be configured to aid in the positioning and/or maintenance of one or more external devices 500 on the patient's skin (e.g. at a location proximate an implanted delivery device 200).

Apparatus 10 can include one or more component positioning devices, such as patient attachment device 70 shown in FIG. 1A, that is used to attach one or more components of external system 50 to a location on or at least proximate the patient. Patient attachment device 70 can comprise one or more elements configured to attach one or more external devices 500 and/or programmer 600 at one or more locations on or proximate the patient's skin, that are relatively close to one or more delivery devices 200 that have been implanted in the patient. Patient attachment device 70 can comprise a component selected from the group consisting of: belt; belt with pockets; belt with adhesive; adhesive; strap; strap with pockets; strap with adhesive shoulder strap; shoulder band; shirt; shirt with pockets; clothing; clothing with pockets; epidural electronics packaging; clip (e.g. a clip that can be adhesively attached to the patient's skin); bracelet; wrist band; wrist watch; anklet; ankle bracelet; knee strap; knee band; thigh strap; thigh band; necklace; hat; headband; collar; glasses; goggles; earpiece; behind-the-earpiece; and combinations of these.

Apparatus 10 can comprise a device configured to operate (e.g. temporarily operate) one or more delivery devices 200, such as trialing interface 80 shown in FIG. 1A. Trialing interface 80 can be configured to wirelessly deliver power to a delivery device 200, wirelessly deliver data to a delivery device 200, and/or wirelessly receive data from a delivery device 200.

In some embodiments, apparatus 10 comprises a diagnostic assembly, diagnostic assembly 62 shown in FIG. 1A. In some embodiments, programmer 600 and/or implantable controller 250 comprise all or a portion of diagnostic assembly 62. Diagnostic assembly 62 can be configured to assess, monitor, determine and/or otherwise analyze patient information and/or delivery device 200 information, such as when one or more stimulation elements 260, functional elements 299, and/or functional elements 599 are configured as a sensor configured to record patient information (e.g. patient physiologic information and/or patient environment information) and/or apparatus 10 information (e.g. delivery device 200 information) as described herein.

In some embodiments, one or more stimulation elements 260 comprise a stimulation element configured to deliver energy (e.g. one or more electrodes configured to deliver monopolar or bipolar electrical energy) to tissue, and controller 250 is configured to control the energy delivery, such as to control one or more stimulation parameters. Each of these stimulation parameters can be held relatively constant, and/or varied, such as a variation performed in a continuous or intermittent manner. In some embodiments, one or more stimulation parameters are varied in a random or pseudo-random (hereinafter “random”) manner, such as a variation performed by apparatus 10 using a probability distribution as described in applicant's co-pending U.S. patent application Ser. No. 16/104,829, titled “Apparatus with Enhanced Stimulation Waveforms”, filed Aug. 17, 2018 [Docket nos. 47476-708.301; NAL-014-US]. In some embodiments, stimulation (e.g. stimulation comprising high frequency and/or low frequency signal components) is varied randomly to eliminate or at least reduce synchrony of neuronal firing with the stimulation signal (e.g. to reduce paresthesia or other patient discomfort). In some embodiments, one or more stimulation elements 260 comprise a stimulation element configured to stimulate a target (e.g. nerve tissue such as spinal nerve tissue and/or peripheral nerve tissue). The amount of stimulation delivered to the target can be controlled by varying a parameter selected from the group consisting of: stimulation element 260 size and/or configuration (e.g. electrode size and/or configuration); stimulation element 260 shape (e.g. electrode shape, magnetic field generating transducer shape or agent delivering element shape); shape of a generated electric field; shape of a generated magnetic field; stimulation signal parameters; and combinations of these.

In some embodiments, controller 250 is configured to produce a stimulation signal comprising a waveform or a waveform pattern (hereinafter stimulation waveform), for one or more stimulation elements 260 configured as an energy delivering stimulation element (e.g. such that one or more stimulation elements 260 deliver stimulation energy comprising or at least resembling that stimulation waveform). Controller 250 can produce a stimulation signal comprising a waveform selected from the group consisting of: square wave; rectangle wave; sine wave; sawtooth; triangle wave (e.g. symmetric or asymmetric); trapezoidal; ramp; waveform with exponential increase; waveform with exponential decrease; pulse shape which minimizes power consumption; Gaussian pulse shape; pulse train; root-raised cosine; bipolar pulses; and combinations of these. In some embodiments, controller 250 is configured to produce a stimulation signal comprising a waveform including a combination of two or more waveforms selected from the group consisting of: square wave; rectangle wave; sine wave; triangle wave (symmetric or asymmetric); ramp; waveform with exponential increase; waveform with exponential decrease; pulse shape which minimizes power consumption; Gaussian pulse shape; pulse train; root-raised cosine; bipolar pulses; and combinations of these. In some embodiments, controller 250 is configured to construct a custom waveform (e.g. an operator-customized waveform), such as by adjusting amplitude at specified time steps (e.g. for one or more pulses). In some embodiments, controller 250 is configured to generate a waveform including one or more random parameters (e.g. random timing of pulses or random changes in frequency, rate of change or amplitude).

In some embodiments, controller 250 is configured to provide a stimulation signal comprising waveforms and/or pulses repeated at a frequency (e.g. includes a frequency component) between 1.0 Hz and 50 KHz, such as between 10 Hz and 500 Hz, between 40 Hz and 160 Hz and/or between 5 KHz and 15 KHz. In some embodiments, controller 250 is configured to produce a stimulation signal comprising a frequency between 1 Hz and 1000 Hz, such as a stimulation signal with a frequency between 10 Hz and 500 Hz. In some embodiments, controller 250 is configured to produce a stimulation signal comprising a duty cycle between 0.1% and 99%, such as a duty cycle between 1% and 10% or between 1% and 25%. In some embodiments, controller 250 is configured to produce a stimulation signal comprising a frequency modulated stimulation waveform, such as a stimulation waveform comprising a frequency component (e.g. signal) between 1 kHz and 20 kHz. In some embodiments, controller 250 is configured to produce a stimulation signal comprising a mix and/or modulation of low frequency and high frequency signals, which comprise any of the waveform types, shapes and other configurations. In these embodiments, the stimulation signal can comprise low frequency signals between 1 Hz and 1000 Hz, and high frequency signals between 600 Hz and 50 kHz, or between 1 kHz and 20 kHz. Alternatively or additionally, the stimulation signal can comprise a train of high frequency signals and bursts of low frequency signals, and/or a train of low frequency signals and bursts of high frequency signals. Alternatively or additionally, the stimulation signal can comprise one or more high frequency signals modulated with one or more low frequency signals, such as one or more high frequency signals frequency modulated (FM), amplitude modulated (AM), phase modulated (PM) and/or pulse width modulated (PWM) with one or more low frequency signals. The stimulation signal can cycle among different waveforms shapes at specified time intervals. The stimulation signal can comprise a pseudo random binary sequence (PRBS) non-return-to-zero or return-to-zero waveform, such as with a fixed and/or time-varying pulse width and/or frequency of the pulses.

In some embodiments, implantable system 20 of apparatus 10 is configured to provide paresthesia-reduced (e.g. paresthesia-free) high frequency pain management and rehabilitation therapy (e.g. via delivery of a stimulation signal above 600 Hz or 1 kHz, or other stimulation signal resulting in minimal paresthesia). Apparatus 10 can be configured to provide both low frequency (e.g. <1 kHz) stimulation and high frequency stimulation, such as when providing low frequency stimulation to elicit feedback from a patient during intraoperative or other (e.g. post-implantation) stimulation configuration. For example, trialing interface 80 can be used during an intra-operative titration of stimulation configuration using low frequency stimulation (e.g. to position and/or confirm position of one or more stimulation elements 260, such as to confirm sufficient proximity to target tissue to be stimulated and/or sufficient distance from non-target tissue not to be stimulated). In some embodiments, high frequency stimulation is delivered to reduce pain over extended periods of time, and low frequency stimulation is used in these intraoperative and/or post-implantation titration or other stimulation configuration procedures. Intentional elicitation of paresthesia (e.g. via low frequency stimulation and/or high frequency stimulation) is beneficial during stimulation element 260 (e.g. electrode) implantation because a patient can provide feedback to the implanting clinician to ensure that the stimulation elements 260 are positioned close to the target neuromodulation or energy delivery site. This implantation position-optimizing procedure can advantageously reduce the required stimulation energy due to stimulation elements 260 being closer to target tissue, since a minimum threshold for efficacious stimulation amplitude is proportional to the proximity of stimulation elements 260 to target tissue (e.g. target nerves). The patient can inform the clinician of the sensation of paresthesia coverage, and the clinician can adjust stimulation element 260 position to optimize stimulation element 260 location for efficacious treatment while minimizing unintentional stimulation of non-target tissue (e.g. motor nerves or other nerves which are not causing the patient's pain). These paresthesia-inducing techniques (e.g. using low frequency stimulation and/or high frequency stimulation) can be used during or after implantation of one or more delivery devices 200.

In some embodiments, apparatus 10 is configured to deliver low frequency stimulation energy (e.g. electrical energy comprising a low frequency signal) to stimulate motor nerves, such as to improve tone and structural support (e.g. physical therapy). In these embodiments, apparatus 10 can be further configured to provide high frequency stimulation, such as to treat pain (e.g. suppress and/or control pain). The combined effect can be used not only for pain management but also muscle strengthening and gradual healing of supportive structures. Alternatively or additionally, as described herein, apparatus 10 can be configured to deliver low frequency stimulation energy (e.g. electrical energy) to induce paresthesia, which can also be accompanied by the delivery of high frequency stimulation (e.g. to suppress and/or control pain). In some embodiments, apparatus 10 is configured to deliver low frequency stimulation (e.g. electrical energy comprising a low frequency signal) and burst stimulation, delivered simultaneously or sequentially. The low frequency stimulation and the burst stimulation can be delivered on similar and/or dissimilar stimulation elements 260 (e.g. similar or dissimilar electrode-based stimulation elements 260).

In some embodiments, implantable system 20 of apparatus 10 is configured to perform magnetic field modulation, such as targeted magnetic field neuromodulation (TMFN), electro-magnetic field neuromodulation, such as targeted electro-magnetic field neuromodulation (TEMFN), transcutaneous magnetic field stimulation (TMS), or any combination of these. Each delivery device 200, via one or more of its stimulation elements 260 (e.g. electrodes) can be configured to provide localized (e.g. targeted) magnetic and/or electrical stimulation. Combined electrical field stimulation and magnetic field stimulation can be applied by using superposition, and this combination can reduce the overall energy requirement. In some embodiments, implantable apparatus 10 comprises one or more stimulation elements 260 comprising a magnetic field generating transducer (e.g. microcoils or cuff electrodes positioned to partially surround or otherwise be proximate to one or more target nerves). Stimulation elements 260 comprising microcoils can be aligned with nerves to minimize affecting non-targeted tissue (e.g. to avoid one or more undesired effects to non-target tissue surrounding or otherwise proximate the target tissue). In some embodiments, the target tissue comprises dorsal root ganglia (DRG) tissue, and the non-target tissue comprises ventral root tissue (e.g. when the stimulation energy is below a threshold that would result in ventral root tissue stimulation).

One or more delivery devices 200 can be configured to deliver stimulation energy with a stimulation waveform that varies over time. In some embodiments, one or more stimulation parameters of the stimulation waveform are randomly varied over time, such as by using a probability distribution as described in applicant's co-pending U.S. patent application Ser. No. 16/104,829, titled “Apparatus with Enhanced Stimulation Waveforms”, filed Aug. 17, 2018 [Docket nos. 47476-708.301; NAL-014-US]. Each stimulation waveform can comprise one or more pulses, such as a group of pulses that are repeated at regular and/or irregular intervals. In some embodiments, a pulse can comprise delivery of electrical energy, such as electrical energy delivered in one or more phases (e.g. a pulse comprising at least a cathodic or anodic portion followed by passive capacitive recovery with an optional open circuit time between the first portion and recovery). In some embodiments, a group of pulses is delivered, each pulse comprising an anodic or cathodic portion that can include charge recovery after each pulse, such as charge recovery comprising active (opposite polarity pulse) recovery, and/or passive (capacitive) recovery. In some embodiments, there is no recovery between pulses, but instead active or passive recovery is included at the end of the set of the first (anodic or cathodic) portions. In some embodiments, single or groups of pulses are provided at time-varying modes of repetition (e.g. regular intervals for a period, then a period of irregular intervals) or at regular intervals with occasional (random) spurious pulses inserted (creating a single irregular event in an otherwise regular series). Non-limiting examples of waveform variations include: a variation in frequency (e.g. frequency of one or more signals of the waveform); variation of a signal amplitude; variation of interval time period (e.g. a time period between pulses or a time period between pulse trains); variation of a pulse width; multiple piecewise or continuous variations of one or more stimulation parameters in a single pulse (e.g. multi-step, multi-amplitude in one “super-pulse”); variation of pulse symmetry (e.g. via active drive, passive recovery and/or active-assisted passive recovery); variation of stimulation energy over a time window and/or overlapping time windows; variation of the power in the frequency spectrum of the stimulation waveform; and combinations of these. In some embodiments, apparatus 10 and/or delivery device 200 can be configured to vary a stimulation waveform “systematically” such as a variation performed temporally (e.g. on predetermined similar or dissimilar time intervals) and/or a variation performed based on a parameter, such as a measured parameter that can be based on a signal produced by a sensor of delivery device 200 or another component of apparatus 10. Alternatively or additionally, apparatus 10 and/or delivery device 200 can be configured to vary a stimulation waveform randomly. Random variation shall include discrete or continuous variations that can be selected from a distribution, such as a probability distribution selected from the group consisting of: a uniform distribution; an arbitrary distribution; a gamma distribution; a normal distribution; a log-normal distribution; a Pareto distribution; a Gaussian distribution; a Poisson distribution; a Rayleigh distribution; a triangular distribution; a statistic distribution; and combinations of these. Random pulses or groups of pulses can be generated based on randomly varying one or more stimulation signal parameters. One or more stimulation parameters can be varied randomly through the use of one or more probability distributions.

Apparatus 10 can be configured to stimulate tissue (e.g. stimulate nerve tissue such as tissue of the central nervous system or tissue of the peripheral nervous system, such as to neuromodulate nerve tissue), such as by having one or more delivery devices 200 deliver and/or otherwise provide energy (hereinafter “deliver energy”) and/or deliver an agent (e.g. a pharmaceutical compound or other agent) to one or more tissue locations, such as via one or more stimulation elements 260. In some embodiments, one or more delivery devices 200 deliver energy and/or an agent while receiving power and/or data from one or more external devices 500. In some embodiments, one or more delivery devices 200 deliver energy and/or an agent (e.g. continuously or intermittently) using energy provided by an internal power source (e.g. energy storage assembly 270) without receiving externally supplied power, such as for periods of at least 1 hour, at least 1 day, at least 1 month or at least 1 year. In some embodiments, one or more stimulation parameters are varied (e.g. systematically and/or randomly), during that period.

In some embodiments, apparatus 10 is further configured as a patient diagnostic apparatus, such as by having one or more delivery devices 200 record a patient parameter (e.g. a patient physiologic parameter) from one or more tissue locations, such as while receiving power and/or data from one or more external devices 500. In some embodiments, during its use, one or more delivery devices 200 at least receives power from one or more external devices 500 (e.g. with or without also receiving data). Alternatively or additionally, one or more patient parameters can be recorded by an external device of apparatus 10, such as via a programmer 600 and/or an external device 500.

Apparatus 10 can be configured as a patient information recording apparatus, such as by having one or more delivery devices 200 and/or one or more external devices 500 record patient information (e.g. patient physiologic information and/or patient environment information). In some embodiments, one or more delivery devices 200 and/or one or more external devices 500 further collect information (e.g. status information or configuration settings) of one or more of the components of apparatus 10.

In some embodiments, apparatus 10 is configured to deliver stimulation energy to tissue to treat pain. In particular, apparatus 10 can be configured to deliver stimulation energy to tissue of the spinal cord and/or tissue associated with the spinal cord (“tissue of the spinal cord”, “spinal cord tissue” or “spinal cord” herein), the tissue including roots, dorsal root, dorsal root ganglia, spinal nerves, ganglia, and/or other nerve tissue. The delivered energy can comprise energy selected from the group consisting of: electrical energy; magnetic energy; electromagnetic energy; light energy such as infrared light energy, visible light energy and/or ultraviolet light energy; mechanical energy; thermal energy such as heat energy and/or cryogenic energy; sound energy such as ultrasonic sound energy (e.g. high intensity focused ultrasound and/or low intensity focused ultrasound) and/or subsonic sound energy; chemical energy; and combinations of these. In some embodiments, apparatus 10 is configured to deliver to tissue energy in a form selected from the group consisting of: electrical energy such as by providing a controlled (e.g. constant or otherwise controlled) electrical current and/or voltage to tissue; magnetic energy (e.g. magnetic field energy) such as by applying controlled current or voltage to a coil or other magnetic field generating element positioned proximate tissue; and/or electromagnetic energy such as by providing both current to tissue and a magnetic field to tissue. A coil or other magnetic field generating element can surround (e.g. at least partially surround) the target nerve. Alternatively, or additionally, the magnetic energy can be applied externally and focused to specific target tissue via an implant comprising a coil and/or ferromagnetic materials. In some embodiments, the magnetic energy is configured to induce the application of mechanical energy. Delivered energy can be supplied in one or more stimulation waveforms, each waveform comprising one or more pulses of energy, as described in detail herebelow.

In some embodiments, apparatus 10 is configured as a stimulation apparatus in which external system 50 transmits a power signal to one or more delivery devices 200, and the one or more delivery devices 200 deliver stimulation energy to tissue with a stimulation signal (also referred to as a stimulation waveform), with the power signal and the stimulation signal having one or more different characteristics (e.g. as described herebelow). The power signal can be modulated with data (e.g. configuration or other data to be sent to one or more delivery devices 200). In these embodiments, the characteristics of the stimulation signal delivered (e.g. amplitude, frequency, duty cycle and/or pulse width), can be independent (e.g. partially or completely independent) of the characteristics of the power signal transmission (e.g. amplitude, frequency, phase, envelope, duty cycle and/or modulation). For example, the frequency and modulation of the power signal can change without affecting those or other parameters of the stimulation signal, and/or the parameters of the stimulation signal can be changed (e.g. via programmer 600), without requiring similar or any changes to the power signal. In some embodiments, implantable system 20 is configured to rectify the received power signal, and to produce a stimulation waveform with entirely different characteristics (e.g. amplitude, frequency and/or duty cycle) from the rectified power signal. Each delivery device 200 can comprise an oscillator and/or controller configured to produce the stimulation signal. In some embodiments, one or more delivery devices 200 is configured to perform frequency multiplication, in which multiple signals are multiplexed, mixed, added, and/or combined in other ways to produce a broadband stimulation signal.

In some embodiments, apparatus 10 is configured to treat a patient disease or disorder selected from the group consisting of: chronic pain; acute pain; migraine; cluster headaches; urge incontinence; pelvic dysfunction such as overactive bladder; fecal incontinence; bowel disorders; tremor; obsessive compulsive disorder; depression; epilepsy; inflammation; tinnitus; hypertension; heart failure; carpal tunnel syndrome; sleep apnea; obstructive sleep apnea; dystonia; interstitial cystitis; gastroparesis; obesity; mobility issues; arrhythmia; rheumatoid arthritis; dementia; Alzheimer's disease; eating disorder; addiction; traumatic brain injury; chronic angina; congestive heart failure; muscle atrophy; inadequate bone growth; post-laminectomy pain; liver disease; Crohn's disease; irritable bowel syndrome; erectile dysfunction; kidney disease; and combinations of these.

Apparatus 10 can be configured to treat heart disease, such as heart failure of a patient. In these embodiments, stimulation of the spinal cord can be performed. Apparatus 10 can be configured to pace and/or defibrillate the heart of a patient. One or more stimulation elements 260 can be positioned proximate cardiac tissue and deliver a stimulation signal as described herein (e.g. based on power and/or data received by implantable system 20 from external system 50). The stimulation signal can be used to pace, defibrillate and/or otherwise stimulate the heart. Alternatively or additionally, apparatus 10 can be configured to record cardiac activity (e.g. by recording EKG, blood oxygen, blood pressure, heart rate, ejection fraction, wedge pressure, cardiac output, lung impedance and/or other properties or functions of the cardiovascular system via a sensor-based element 260, and/or other sensor of apparatus 10), such as to determine an onset of cardiac activity dysfunction or other undesired cardiac state. In some embodiments, apparatus 10 is configured to both record cardiac or other information and deliver a stimulation signal to cardiac tissue (e.g. stimulation varied or otherwise based on the recorded information). For example, apparatus 10 can be configured such that external system 50 transmits power and/or data to implantable system 20. Implantable system 20 monitors cardiac activity, and upon detection of an undesired cardiovascular state, implantable system 20 delivers a pacing and/or defibrillation signal to the tissue that is adjacent to one or more stimulation elements 260 configured to deliver a cardiac stimulation signal.

As described hereabove, apparatus 10 can comprise an implantable system 20 which can include one or more delivery devices 200. Each delivery device 200 comprises a housing 210 and one or more leads 265, such as leads that are operator-attachable to housing 210 (e.g. attached in an implantation procedure), and/or fixedly attached to housing 210 (e.g. attached during a manufacturing process of device 200).

Each lead 265 comprises one or more stimulation elements 260. Stimulation elements 260 can comprise electrical energy delivery elements (e.g. electrodes), electromagnetic energy delivery elements, light delivery elements, sounds delivery elements, pharmaceutic drug and/or other agent delivery elements (e.g. needles and/or catheters), and/or other stimulation elements. Each lead 265 can be positioned (e.g. implanted by a clinician of the patient) to subsequently stimulate tissue (e.g. deliver stimulation energy and/or deliver a stimulating agent to stimulate tissue), such as when stimulation elements 260 are positioned in one or more anatomical locations to stimulate particular nerve tissue, such as to treat pain and/or provide another therapy to a patient. One or more stimulation elements 260 (e.g. positioned on one or more leads 265) can be positioned in the patient to perform spinal cord stimulation (SCS). Precise positioning of the stimulation elements 260 in the patient is related to the efficacy of the treatment (e.g. related to the amount of pain relief achieved).

Apparatus 10 can be constructed and arranged to prevent or at least reduce (“reduce” herein) migration of each lead 265 over time, where such migration can comprise efficacy of stimulation energy delivery by the stimulation elements 260. Alternatively or additionally, apparatus 10 can be constructed and arranged to detect lead 265 migration, such as to detect magnitude of lead 265 migration and/or relative positioning changes of lead 265 after the migration.

Apparatus 10 can be configured to compensate for migration of one or more leads 265. For example, if a lead 265 migration is detected, one or more events can be performed, such as: an adjustment of stimulation energy delivery (e.g. pattern) delivered by the associated lead 265 (e.g. a stimulation pattern adjustment based on the detected migration); and/or a repositioning of lead 265 (e.g. in a surgical or other clinical procedure).

In some embodiments, each lead 265 can further comprise one or more electrodes 2600, such as electrodes that are used by algorithm 15 of apparatus 10 to assess migration of one or more leads 265 (e.g. amount of migration of one or more portions of a lead 265 from a first instance in time to a second instance in time) and/or identify the anatomical location of one or more leads 265 (e.g. identify the anatomical location of one or more portions of a lead 265 at the current instance in time). In some embodiments, one or more stimulation elements 260 of one or more leads 265 comprise an electrode that is configured to function as an electrode 2600 (e.g. a stimulation element 260 and the associated electrode 2600 are the same electrode). Alternatively or additionally, in some embodiments one or more stimulation elements 260 of one or more leads 265 are a non-electrode stimulation element, such as a non-electrode stimulation element selected from the group consisting of: a light delivery element (e.g. a lens or other optical component), a sound delivery element (e.g. an ultrasound delivery transducer); an electromagnetic energy delivery element; a pharmaceutical drug or agent delivery element (e.g. a needle, an opening in a catheter, and the like); and combinations thereof. In these embodiments, lead 265 can further comprise one or more electrodes 2600, such as one or more electrodes 2600 that are each positioned on lead 265 in close proximity to a non-electrode stimulation element 260, and/or located on another portion of lead 265. Whether electrodes 2600 are the same component as the stimulation elements 260, or whether stimulation elements 260 and electrodes 2600 comprise separate components positioned on a lead 265, electrodes 2600 can be used by algorithm 15 of apparatus 10 to determine the migration and/or anatomical location of stimulation elements 260 and/or one or more other portions of the associated lead 265, such as is described herein in reference to applicant's co-pending International PCT Patent Application Serial Number PCT/US2020/066901, titled “Systems with Implanted Conduit Tracking”, filed Dec. 23, 2020 [Docket nos. 47476-716.601; NAL-022-PCT].

In some embodiments, apparatus 10 is configured to steer current delivered by one or more stimulation elements 260. In these embodiments, apparatus 10 can be configured with a set of amplitudes for the anodes and cathodes of a set of stimulation elements 260. The relative amplitudes of the anodes and cathodes can be set to deliver energy to a target anatomical location. The target anatomical location can be set using paresthesia mapping, such as by delivering a stimulation waveform that includes tonic stimulation configured to cause paresthesia. As a patient changes their posture, the distance changes between the patient's spinal cord and stimulation elements 260 of one or more implanted leads 265. This changing posture will cause the electric fields generated by elements 260 to change, such as movement that causes the point of maximum field intensity, referred to as a “centroid”, to no longer being present at the target anatomical location. In some embodiments, apparatus 10 is configured to cause the current steering amplitudes to change in either a deterministic or random manner. The configuration would result in a larger anatomical area receiving stimulation energy, such as to compensate for variations caused by patient movement (e.g. posture change), and/or to compensate for neural fatigue. In some embodiments, a user interface (e.g. a user interface of a programmer 600) can display an image of an anatomical area in which an operator of apparatus 10 (e.g. the patient) can make a selection, and apparatus 10 is configured to traverse this area (e.g. either deterministically or randomly). In some embodiments, apparatus 10 is configured to collect and/or process posture data, such as to automatically readjust the centroid based on the patient's current posture.

In some embodiments, apparatus 10 is configured to steer current as described herein in reference to FIGS. 2-19.

Referring now to FIG. 2, a schematic of electronic circuitry configured to steer stimulation current is illustrated, consistent with the present inventive concepts. Current steering circuitry can include high and low side current sources, such as high and low side digital-to-analog converters, or DACs (e.g. PDACs and NDACs). Using both P and N sources can occupy a significant “area” of an integrated circuit, as well as can require significant amounts of power in order to operate (e.g. driving both the P and N sources requires twice the power compared to driving one or the other). In some embodiments, a single set of DACs can be configured to steer stimulation current. In FIG. 2, a single set of PDACs is included. In alternative embodiments, a single set of NDACs can be used.

When stimulating with just two DACs as shown, the currents Iₐ and I_{b} will split into I1+I2, and I3+I4, respectively, each as shown, the splitting based on the impedances of the associated tissue volumes receiving the current. The object of the provided current steering is to drive a precise amount of current through a first pair of stimulation elements (e.g. electrodes), elements 260a and 260c, and a second pair of stimulations elements (e.g. electrodes), elements 260b and 260d. In order to provide enhanced precision in steering current, one or more switches can be included on the return line (non-driven) of a stimulation element 260. For example, as shown in FIG. 2, switches S1 and S2 can be included, each switch configured to disconnect stimulation elements 260c and 260d, respectively, from circuit ground. In some embodiments, the one or more switches are each enabled for a period of time proportional to the current ratio that is to flow through the associated stimulation element 260.

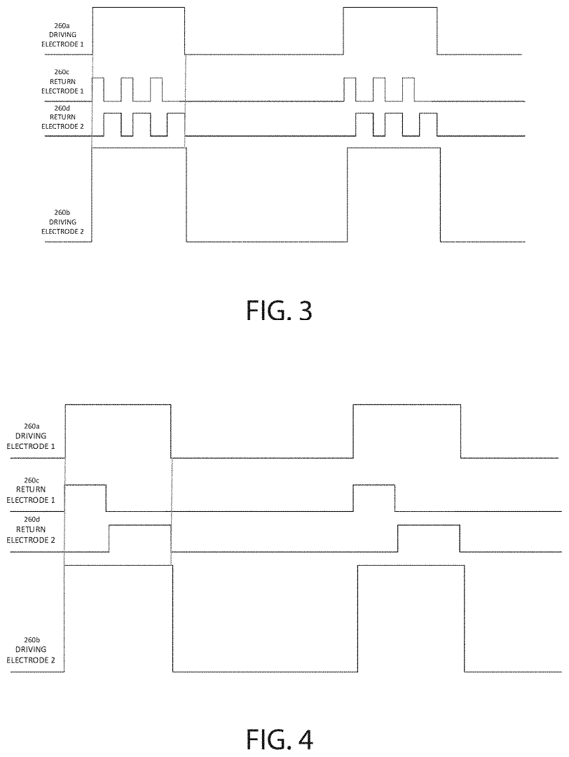

Apparatus 10 can be configured in various multiplexing schemes to achieve desired current steering. For example, in FIG. 3, a configuration in which current proportions of 40% and 60% are achieved by the delivery of two sets of three pulses each, where stimulation element 260c receives three pulses, each comprising 13.3% of the current delivered, and stimulation element 260d receives three pulses, each comprising 20% of the current delivered. In FIG. 4, a configuration in which current proportions of 40% and 60% are achieved by the delivery of two sets of a single pulse each, where stimulation element 260c receives one pulse comprising 40% of the current delivered, and stimulation element 260d receives one pulse comprising 60% of the current delivered. In these single source multiplexed configurations, neurons that are depolarized by the delivered stimulation pulses receive a stimulation that is substantially equivalent to a non-multiplexed configuration (e.g. using simultaneous P & N sources). In some embodiments, apparatus 10 is configured to rapidly switch between the return electrodes (e.g. switching at a rate between 1 kHz and 1 MHz, such as a rate between 1 kHz and 10 kHz, between 10 kHz and 100 kHz, between 100 kHz and 1 MHz, and/or between 500 kHz and 1 MHz).

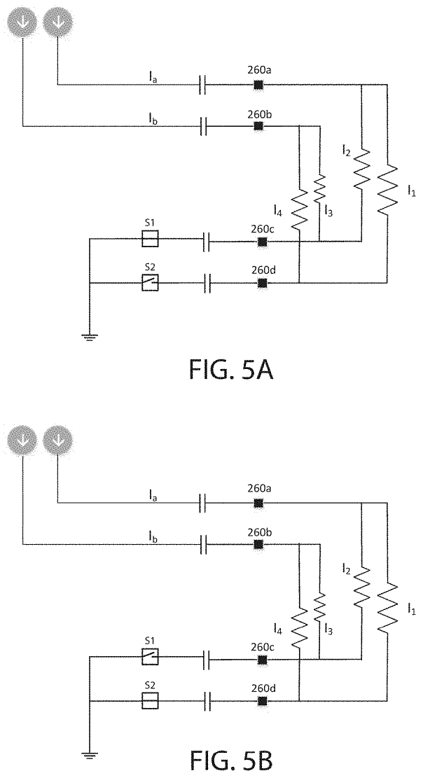

In these embodiments, apparatus 10 can be configured to provide charge balance, such as by ensuring that each stimulation element 260 has an equal amount of charge flowing through it in each direction (e.g. each element 260 should on average be at net zero charge). In configurations that do not include both P and N sources, apparatus 10 can utilize time division multiplexing to supply current to the stimulation elements, such as is shown in FIGS. 5A and 5B. A switch, S1, is enabled (i.e. closed) for a duration proportional to the ratio of current that is to flow through switch S1 and the connected stimulation elements 260. As described hereabove, during the recovery phase one stimulation element 260 would be connected for 10% of the time, and the second stimulation element 260 for 90% of the time. Since this period is for the recovery phase, and no neural stimulation is expected to occur, a simple multiplexing scheme should suffice. Although two current sources (Ia, Ib) are shown in FIGS. 5A and 5B, in some embodiments, a single source (Ia+Ib) can be used. An advantage in using two current sources is that the total current (Ia+Ib) using the two sources is the same as is used during the stimulating phase, thereby reducing any charge mismatch.

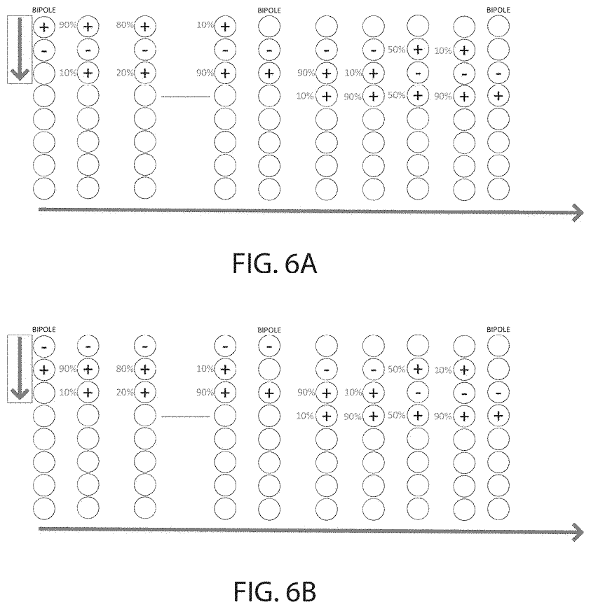

In some embodiments, apparatus 10 implements current steering to stimulate tissue locations along the spinal cord, or other nerve tissue, by varying the relative amplitudes of the anodes and cathodes of the electrode-based stimulation elements 260. When programming apparatus 10 for current steering, an operator (e.g. the clinician of the patient) can utilize feedback received from the patient regarding “paresthesia coverage” (e.g. patient feedback regarding where paresthesia occurred) in order to determine relative contributions and stimulation element 260 configurations. For example, two potential arrangements are shown in FIGS. 6A and 6B. As the operator engages an “up-down” control (e.g. button) to traverse the target space, the configuration and duration percentages can change as shown. Alternatively or additionally, apparatus 10 can be configured to implement current steering to target one or more particular anatomic locations, such as when delivery device 200 is configured to stimulate tissue on the superior-side of T9 vertebral space.

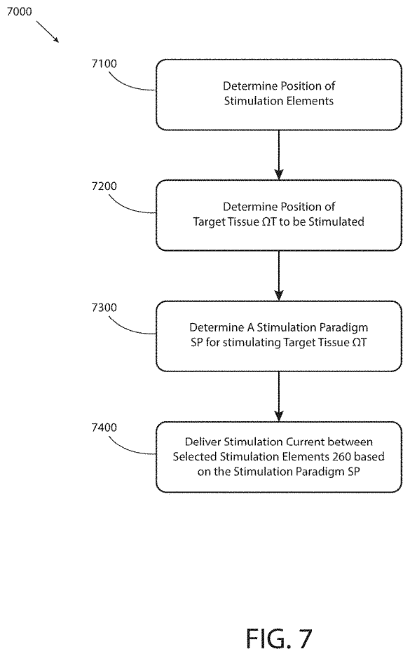

Referring now to FIG. 7, a flow chart of method of steering current is illustrated, consistent with the present inventive concepts. Method 7000 of FIG. 7 can be used by apparatus 10 to steer current to a volume of tissue to be stimulated, target tissue Ω_{T}, such as to treat back pain or other pain of the patient, and/or otherwise to provide a therapy to the patient. Method 7000 is described using apparatus 10 and its components of the present inventive concepts.

In Step 7100, the implanted position of two or more stimulation elements 260 of one or more delivery devices 200 is determined. The two or more stimulation elements 260 can be positioned, on a single lead 265, two leads 265, or three or more leads 265. In some embodiments, the implanted positions of elements 260 is determined by algorithm 15, as described herein and/or in applicant's co-pending application International PCT Patent Application Serial Number PCT/US2020/066901, titled “Systems with Implanted Conduit Tracking”, filed Dec. 23, 2020 [Docket nos. 47476-716.601; NAL-022-PCT]. Alternatively or additionally, the implanted location of elements 260 can be determined by analysis of one or more images (e.g. X-ray, ultrasound, and/or other images) of the implanted elements 260, such as an analysis performed manually by an operator (e.g. clinician) of apparatus 10 and/or in an automated (e.g. semi or fully automated) fashion by algorithm 15. Alternatively or additionally, the implanted position of elements 260 can be entered manually by the implanting clinician or other operator of apparatus 10 with knowledge of the implanted positions.

In Step 7200, the position of target tissue Ω_{T} is determined, such that target tissue Ω_{T} comprises one or more volumes of tissue to be stimulated. In some embodiments, target tissue Ω_{T} includes one or more locations in which the patient has reported experiencing pain. In some embodiments, target tissue Ω_{T} includes one or more locations of an organ or other tissue to which stimulation provides a therapeutic benefit. The region in which target tissue can be stimulated efficiently depends on the implant location of the leads 265, in other words on the implant location of the stimulation elements 260.

In Step 7300, a stimulation paradigm SP is determined by algorithm 15 of apparatus 10, such as a stimulation paradigm which includes identification of a set of multiple stimulation elements 260 to source and/or sink stimulation current, and the parameters (e.g. amplitudes, pulse widths, waveform shapes, and the like) of the currents being sourced and sinked by each element 260. In some embodiments, one or more stimulation elements 260 is configured as an anode during one or more portions of stimulation delivery, and as a cathode during one or more different portions of stimulation delivery.

In Step 7400, one or more delivery devices 200, via one or more leads 265 and its associated stimulation elements 260, delivers the stimulation energy to the patient based on the stimulation paradigm SP.

In Step 7300, algorithm 15 can determine stimulation paradigm SP by performing an analysis, such as an FEA, based on the implanted positions of elements 260 as determined in Step 7100, as well as the location of target tissue Ω_{T} determined in Step 7200. In some embodiments, an FEA analysis utilized by algorithm 15 is similar to that described herein in reference to Formulation Section 3 below.

Alternatively or additionally, algorithm 15 can determine stimulation paradigm SP based on comparison of the implanted positions of elements 260 as determined in Step 7100, as well as the location of target tissue Ω_{T} determined in Step 7200, to a library of models of stimulation paradigms SP, such as a library of models included in library 16 described herein. In some embodiments, one or more models of library 16 are based on an FEA analysis, such as is described herein. In some embodiments, two or more models of library 16 are selected, and algorithm 15 averages or otherwise mathematically combines the associated stimulation paradigms (e.g. SP₁, SP₂, and the like) to determine the stimulation paradigm SP to be delivered in Step 7400.

In some embodiments, algorithm 15 differentiates different types of tissue, such as to differentiate two or more tissue types selected from the group consisting of: bone; a vertebra; grey matter; white matter; dura; and combinations of these.

In some embodiments, one or more steps of method 7000 of FIG. 7 include one or more stimulation elements 260 delivering stimulation energy that causes paresthesia, such as to get patient feedback regarding an area being stimulated. In these embodiments, algorithm 15 can utilize this patient feedback in order to determine a stimulation paradigm SP.

### Section 1—Introduction

The below text describes methods and apparatus 10 configurations for determining stimulation element 260 excitation patterns for steering current toward a target region.

### Section 2—Geometry

A target region to be stimulated comprises a 3D geometry of patient tissue.

### Section 3—Formulation

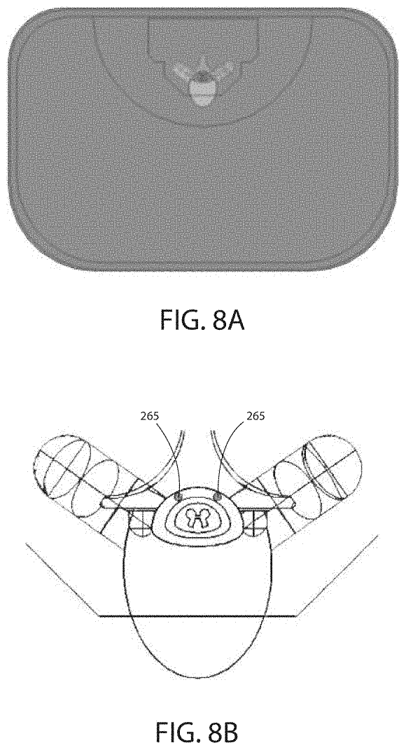

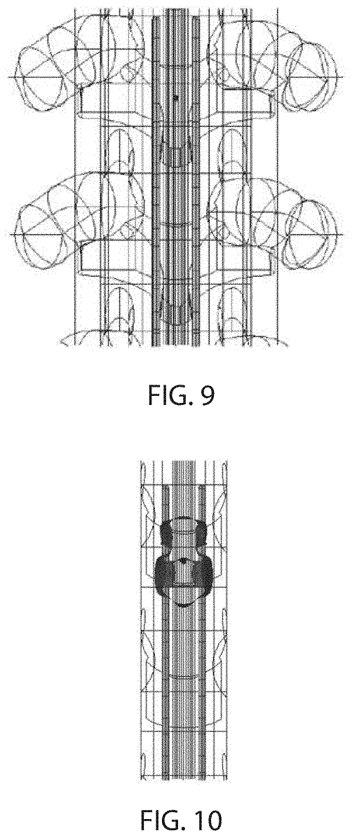

FIGS. 8A and 8B illustrate a sectional anatomical view, and a magnified sectional anatomical view, respectively, of a patient's spinal cord, with implanted leads 265 and stimulation elements 260 as shown. As described herein, apparatus 10 can comprise an algorithm 15 that uses a formulation to steer current delivered by electrode-based stimulation elements 260 into one or more target regions, such as target region Ω_{T}, of a tissue domain Ω. The voltage (U) distribution in the domain Ω is defined by:

∇·σ∇U=0inΩ (1)

Letting fₓᵢ be the voltage field defined over the domain such that voltage for i^{th} stimulation element 260 is set to 1 (vᵢ=1) and all other stimulation elements 260 are set to circuit ground (also referred to as simply “ground” herein). Hence, any voltage distribution in the domain Ω can be represented as a linear combination of voltage field fₓᵢ, where:

f_{\acute{x}}=Σᵢvᵢf_{\acute{x}i} (2)

The corresponding current density is given by:

σ∇f_{\acute{x}}=σΣᵢvᵢ∇f_{\acute{x}i} (3)

where σ is the conductivity tensor defined over the entire domain Ω. In the other embodiments, the conductivity tensor comprises a diagonal form:

$$σ = [ \begin{matrix}\begin{matrix}{σ}^{1} 00 ; & 0 {σ}^{2} 0 ;\end{matrix} & 00 {σ}^{3}\end{matrix} ] = ( \begin{matrix}{σ}^{1} & 0 & 0 \\ 0 & {σ}^{2} & 0 \\ 0 & 0 & {σ}^{3}\end{matrix} ) .$$

In some embodiments, the conductivity tensor a comprises a symmetric positive definite matrix. The following formulation is described using a diagonal form.
It can be desirable to maximize the ratio of integration of square of current density in the target region Ω_{T} with respect to the integration of square of current density in the entire domain Ω. The optimization problem can be defined as:

$$\begin{matrix}{max}_{{v}_{i}} \text{:} \frac{\int \int {\int }_{{Ω}_{T}} {‖ σ ∇ {f}_{{x}^{′}} ‖}^{2}}{\int \int {\int }_{Ω} {‖ σ ∇ {f}_{{x}^{′}} ‖}^{2}} & ( 4 ) \\ {max}_{{v}_{i}} \text{:} \frac{\int \int {\int }_{{Ω}_{T}} {‖ σ {\sum }_{i} {v}_{i} ∇ {f}_{{x}^{′} i} ‖}^{2}}{\int \int {\int }_{Ω} {‖ σ {\sum }_{i} {v}_{i} ∇ {f}_{{x}^{′} i} ‖}^{2}} & ( 5 )\end{matrix}$$

The components of ∇f_{\acute{x}i} can be considered as (g_{\acute{x}i}¹g_{\acute{x}i}²g_{\acute{x}i}³)^{T}. The optimization problem can then be expressed as:

$${max}_{{v}_{i}} \text{:} \frac{\int \int {\int }_{{Ω}_{T}} {\sum }_{j} {( {σ}^{j} {\sum }_{i} {v}_{i} {g}_{{x}^{′} i}^{j} )}^{2}}{\int \int {\int }_{Ω} {\sum }_{j} {( {σ}^{j} {\sum }_{i} {v}_{i} {g}_{{x}^{′} i}^{j} )}^{2}} \qquad ( 6 )$$

Expanding the term Σⱼ(σ^{j}Σᵢvᵢg_{\acute{x}i}^{j})², the following results,

$${\sum }_{j} {( {σ}^{j} {\sum }_{i} {v}_{i} {g}_{{x}^{′} i}^{j} )}^{2} = {\sum }_{i} {v}_{i}^{2} ( {\sum }_{j} {( {σ}^{j} )}^{2} {( {g}_{{x}^{′} i}^{j} )}^{2} ) + {\sum }_{i} {\sum }_{k \neq i} {v}_{i} {v}_{k} ( {\sum }_{j} {( {σ}^{j} )}^{2} {g}_{{x}^{′} i}^{j} {g}_{{x}^{′} k}^{j} ) \qquad ( 7 )$$

Substituting in our optimization problem, the following results,

$$\begin{matrix}{max}_{{v}_{i}} \text{:} \frac{\begin{matrix}\int \int {\int }_{{Ω}_{T}} [ {\sum }_{i} {v}_{i}^{2} ( {\sum }_{j} {( {σ}^{j} )}^{2} {( {g}_{{x}^{′} i}^{j} )}^{2} ) + \\ {\sum }_{i} {\sum }_{k \neq i} {v}_{i} {v}_{k} ( {\sum }_{j} {( {σ}^{j} )}^{2} {g}_{{x}^{′} i}^{j} {g}_{{x}^{′} k}^{j} ) ]\end{matrix}}{\begin{matrix}\int \int {\int }_{Ω} [ {\sum }_{i} {v}_{i}^{2} ( {\sum }_{j} {( {σ}^{j} )}^{2} {( {g}_{{x}^{′} i}^{j} )}^{2} ) + \\ {\sum }_{i} {\sum }_{k \neq i} {v}_{i} {v}_{k} ( {\sum }_{j} {( {σ}^{j} )}^{2} {g}_{{x}^{′} i}^{j} {g}_{{x}^{′} k}^{j} ) ]\end{matrix}} & ( 8 ) \\ {max}_{{v}_{i}} : \frac{\begin{matrix}{\sum }_{i} {v}_{i}^{2} [ \int \int {\int }_{{Ω}_{T}} {\sum }_{j} {( {σ}^{j} )}^{2} {( {g}_{{x}^{′} i}^{j} )}^{2} ] + \\ {\sum }_{i} {\sum }_{k \neq i} {v}_{i} {v}_{k} [ \int \int {\int }_{{Ω}_{T}} {\sum }_{j} {( {σ}^{j} )}^{2} {g}_{{x}^{′} i}^{j} {g}_{{x}^{′} k}^{j} ]\end{matrix}}{\begin{matrix}{\sum }_{i} {v}_{i}^{2} [ \int \int {\int }_{Ω} {\sum }_{j} {( {σ}^{j} )}^{2} {( {g}_{{x}^{′} i}^{j} )}^{2} ] + \\ {\sum }_{i} {\sum }_{k \neq i} {v}_{i} {v}_{k} [ \int \int {\int }_{Ω} {\sum }_{j} {( {σ}^{j} )}^{2} {g}_{{x}^{′} i}^{j} {g}_{{x}^{′} k}^{j} ]\end{matrix}} & ( 9 )\end{matrix}$$

Equation (9) can be rearranged in matrix form as:

$$\begin{matrix}{max}_{{v}_{i}} \text{:} \frac{{{V}^{′}}^{T} C {V}^{′}}{{{V}^{′}}^{T} {C}_{0} {V}^{′}} & ( 10 ) \\ where , & \\ {V}^{′} = {( {v}_{1} {v}_{2} … {v}_{i} … {v}_{N} )}^{T} & ( 11 ) \\ {C}_{ik} = \int \int {\int }_{{Ω}_{T}} {\sum }_{j} ( {( {σ}^{j} )}^{2} {g}_{{x}^{′} i}^{j} {g}_{{x}^{′} k}^{j} ) & ( 12 ) \\ {C}_{0 ik} = \int \int {\int }_{Ω} {\sum }_{j} ( {( {σ}^{j} )}^{2} {g}_{{x}^{′} i}^{j} {g}_{{x}^{′} k}^{j} ) & ( 13 )\end{matrix}$$

Equation (10) represents a quadradic optimization problem.

∇f_{\acute{x}i}=(g_{\acute{x}i}¹g_{\acute{x}i}²g_{\acute{x}i}³)^{T} represent s the electric field E=(EₓE_{y}E_{z})^{T} in the domain Ω.

The elements Cᵢₖ of the matrix C and C₀ᵢₖ of the matrix C₀ can be calculated from simulation results collected by Applicant as described herein. For example, differential equation (1) is solved (in a simulation tool) to find current density σ∇f_{\acute{x}i}, for each i (equations (2) and (3)). This result (a current density) is post-processed to calculate matrix elements Cᵢₖ and C₀ᵢₖ.

Equation (9) considers the integration of the square of the current magnitude. In some embodiments, the integration of square of the voltage magnitude can be performed (e.g. performed by algorithm 15). In some embodiments, the integration of electrical power can be performed. The formulation described above considers objective function as a ratio of the square of current density integrated over the target region Ω_{T} and over the entire domain Ω. The target region is defined based on the location where the current density should be focused. The target region can be of any shape. In some embodiments, the numerator of the objective function can be replaced by a weighted integration of the square of current density in the complete domain Ω. In some embodiments, these weights can be a constant in the target region Ω_{T}, and decay at locations away from the target region. In some embodiments, the weights decay away from a target point. In some embodiments, the weight distribution can be defined such that the weight is a value of 1 inside the target region Ω_{T}, and the weight is a value of 0 outside the target region Ω_{T}. This weighing scheme can be used in the above described formulation. It should be noted that this weight distribution does not imply that there is uniformity of the field inside the target region Ω_{T}. Apparatus 10 (e.g. via algorithm 15) can be configured to provide a ratio-based objective function that does not match a particular current density nor voltage distribution in the entire domain Ω.

### Section 4—Optimization

In some embodiments, algorithm 15 comprises a non-constrained optimization problem (e.g. not including any constraints on the number of sources or sinks) to determine a stimulation paradigm SP. The stimulation paradigm SP can define which stimulation elements are used to deliver current (e.g. as a cathode and/or as an anode), and at what current levels. From equation (10), it is noted that changing the scale of voltage or current does not change the maximums, and therefore the ratio of the values of current across stimulation elements 260 is the important factor, not the absolute magnitudes. Repeating equation (10):

$${max}_{{v}_{i}} \text{:} \frac{{{V}^{′}}^{T} C {V}^{′}}{{{V}^{′}}^{T} {C}_{0} {V}^{′}} \qquad ( 14 )$$

this is a quadratic optimization problem. This formulation has no constraint on the number of current sources. It also does not restrict an element 260 from being a negative current source.

It can be seen that scaling \acute{V} does not change the value of the objective function. Thus, without loss of generality a scale can be chosen such that:

\acute{V}^{T}C₀\acute{V}=1 (15)

Hence, the optimization problem is now a constrained optimization problem,

$${max}_{{v}_{i}} \text{:} {{V}^{′}}^{T} C {V}^{′} \qquad ( 16 )$$

which is subject to:

\acute{V}^{T}C₀\acute{V}=1 (17)

This constrained optimization method can be solved using the method of Lagrangian multipliers. Consider the Lagrangian function:

L(\acute{V}, λ)=\acute{V}^{T}C\acute{V}−λ(\acute{V}^{T}C₀\acute{V}−1) (18)

The solution is given by evaluating the derivative of the Lagrangian function with respect to \acute{V}:

∇_{\acute{V}}L=2C\acute{V}−2λC₀\acute{V}=0 (19)

C\acute{V}=λC₀\acute{V} (20)

Equation (20) is a generalized eigenvalue problem. The eigenvalues will represent the value of the optimization function and the eigenvectors will represent the solutions \acute{V}. As an example, consider the maximum eigenvalue λₘₐₓ and the corresponding eigenvector \acute{V}ₘₐₓ. Substituting in the optimization function given in equation (16), the following results:

\acute{V}ₘₐₓ^{T}C\acute{V}ₘₐₓ (21)

.=\acute{V}ₘₐₓ^{T}(C\acute{V}ₘₐₓ₎ (22)

.=\acute{V}ₘₐₓ^{T}(λₘₐₓC₀\acute{V}ₘₐₓ) (23)

.=λₘₐₓ(\acute{V}ₘₐₓ^{T}C₀\acute{V}ₘₐₓ) (24)

.=λₘₐₓ (25)

### Section 5—Results

FIG. 9 illustrates an anatomical view of two leads 265 and a target region Ω_{T} including tissue of the patient's spine. The algorithm 15 formulation described hereabove in reference to Sections 3 and 4 was verified (for a target region Ω_{T} assumed to lie between the second and third stimulation elements 260 of a lead 265. This target region Ω_{T} is located centrally between both leads 265, shown as a focus point FP1 in FIG. 9.

The elements of C and C₀ matrices as described in equations (12) and (13) were calculated from the simulation results. The optimization problem was solved to obtain the stimulation element 260 voltages corresponding to the maximum eigenvalue.

### Verification of Results

Solving of the optimization problem of Section 4 results in the value of electrodes voltages \acute{V} and the corresponding ratio λₘₐₓ square of current density integrated over the target region Ω_{T} with respect to the square of current density integrated over the complete domain Ω. The electrode currents Í can be calculated from the voltage values \acute{V} and the conductance matrix Y, using the relation Í=Y\acute{V}.

To verify these optimization results, applicant has conducted simulation studies (e.g. studies to solve or otherwise establish a computational model). The currents and voltages which were obtained by solving the optimization problem were specified in a computational model. Currents were specified for stimulation elements 260 configured as sources, and grounded elements 260 were specified at zero voltage. The value of objective function was then calculated from the simulation results of the computational model. This value matched closely with the objective function value obtained from algorithm 15 of apparatus 10.

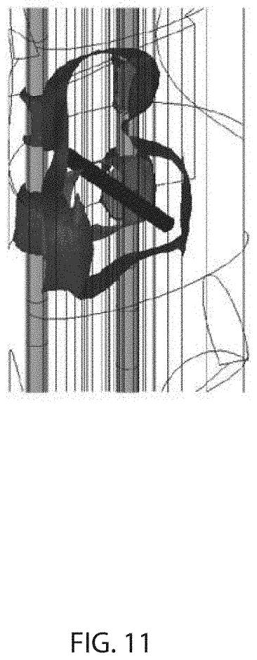

These results can also be verified visually. The surface contour of the norm of the current density is shown in FIG. 10, a surface contour plot for current density in an X-Z view. The current density is seen to concentrate near the contacts closest to the target region Ω_{T} (shown in black). The current steering is also shown in FIG. 11, a magnified view of a surface contour plot for current density.

### Section 6—Optimization Problem With Floating Stimulation Elements 260

The formulation of algorithm 15 described hereabove comprises an optimization problem without any constraints on current sources. In some embodiments, algorithm 15 can include a constraint in which one or more stimulation elements 260 are left “floating” (i.e. disconnected). Considering the current-voltage relationship by way of the conductance matrix Y,

Í=Y\acute{V} (26)

The Í is a vector specifying currents flowing through each contact. A positive value in the vector indicates amount of current sourced from a stimulation element 260, and a negative value indicates amount of current sink into a stimulation element 260. The Y matrix is a square matrix with dimension equal to number of electrodes (N).

Current does not source or sink through a floating stimulation element 260. This condition is equivalent to setting the corresponding entries in Í to 0. Letting the corresponding sub-matrix of Y be Y_{f}:

\acute{0}=Y_{f}\acute{V} (27)

If a quantity of m contacts are floating, then Y_{f} will be a (N−m×N) matrix. Considering the singular value decomposition (SVD) of the matrix Y_{f}:

Y_{f}=AΣB^{T} (28)

The last m columns of Σ will be zeros. Also, B is an orthonormal matrix. Hence, considering \acute{V} to be the linear combination of the last m columns of B, will satisfy equation (27). Letting the sub-matrix formed by these last m columns be defined as Bₛ=B(1: N, N−m+1: N), then the linear combination of these columns can be represented as:

\acute{V}=Bₛα (29)

Substituting equation (29) in the optimization problem, the following results:

$$\begin{matrix}{max}_{{v}_{i}} \text{:} \frac{{{V}^{′}}^{T} C {V}^{′}}{{{V}^{′}}^{T} {C}_{0} {V}^{′}} & ( 30 ) \\ {max}_{α} \frac{{α}^{T} {B}_{s}^{T} {CB}_{s} α}{{α}^{T} {B}_{s}^{T} {C}_{0} {B}_{s} α} = \frac{{α}^{T} D α}{{α}^{T} {D}_{0} α} & ( 31 )\end{matrix}$$

which is subject to:

\acute{V}^{T}C₀\acute{V}=α^{T}D₀α=1 (32)

Y_{f}\acute{V}=0 (33)

Equation (30) represents another constrained quadratic optimization problem, which can be solved using the method of Lagrangian multipliers as described earlier. Solving this optimization problem is equivalent to solving the generalized eigenvalue problem:

Da=λD₀α (34)

The maximum eigenvalue (λₘₐₓ) will be the maximum value of the optimization function and the corresponding eigenvector αₘₐₓ will represent the optimum value of the linear combiners. The stimulation element 260 voltage values are given by:

Vₘₐₓ=Bₛαₘₐₓ (35)

### Section 7—Mixed Formulation

The optimization problem of algorithm 15 described hereabove in Section 4 considers a set of all stimulation elements 260 (e.g. a set of 16 elements 260 that are equally distributed between two leads 265). In some embodiments, such as to reduce computational complexity, a subset of stimulation elements 260 are considered for energy delivery by algorithm 15, such as when half the elements 260 on each of two leads are considered (e.g. 4 of 8 elements on each lead are considered, for a total of 8 elements 260 being considered). The non-considered contacts, for example 8 elements 260, 4 on each lead 265, are modeled as if they are floating. Of the 8 elements 260 being considered for energy delivery, the following constraints can be applied: at most 4 elements 260 are configured to source current (Iᵢ>0); at least one element 260 is configured to be grounded (Vᵢ=0); and the remaining elements 260 are configured to be either a current sink or floating (Vᵢ=0 or Iᵢ=0). The possible combinations from these constraints are:

Total combinations ⁸C₄(2⁴⁻¹)+⁸C₃(2⁵−1)+⁸C₂(2⁶−1)+⁸C₁(2⁷−1)

Total combinations=1050+1736+1764+1016

Total combinations=5566

In the first term, 8C₄ gives the total number of ways in which 4 stimulation elements 260 can be selected as current sources out of the selected 8 stimulation elements 260. (2⁴−1) gives the total number of possible combinations of setting elements 260 to ground. Apart from source and ground, the remainder of the elements 260 will be considered as floating. The second term uses the same logic for counting the combinations, but with three current sources. The total 5566 combinations correspond to different configurations for which optimization needs to be performed. Each configuration specifies a mixture of sourcing, ground, and floating element 260 constraints on optimization.

In some embodiments, configurations with a single lead 265 can be used with algorithm 15 considering all the elements 260 (e.g. a total of 8 elements 260) for optimization on that single lead 265.

In some embodiments, configurations including two leads 265 can be used with algorithm 15 maintaining constraints of sourcing, grounding, and floating of each of the elements 260.

In some embodiments, only anodic current sources are considered for sourcing elements 260. In some embodiments, only cathodic current sources are considered for sourcing elements 260. Embodiments in which only anodic or only cathodic sources are used simplifies and reduces hardware requirements of apparatus 10.

### Constraint on Current Sources

As described hereabove in Section 6, a floating contact (Iᵢ=0) constraint can be accommodated in the generalized eigenvalue formulation. In order to add the constraint Iᵢ≥0 for a selected set of elements 260, the formulation needs to be modified as follows:

$${max}_{α} \text{:} \frac{{α}^{T} D α}{{α}^{T} {D}_{0} α} \qquad ( 36 )$$

which is subject to:

YₛBₛα≥\acute{0} (37)

where Yₛ is a sub-matrix of conductance matrix Y. Iᵢ≥0. Yₛ is constructed by selecting rows in the Y matrix corresponding to the selected current sources (Iᵢ≥0).

In the above formulation, it can be seen that any scale of the solution to the optimization problem α* does not change the value of the objective function. Furthermore, if an a is in the feasible region, then any positive scale of α also lies in the feasible region. Without loss of generality, a positive scale of α can be chosen such that α^{T}D₀α=1. Thus, the above optimization problem can be restated as:

$${max}_{α} \text{:} {α}^{T} D α \qquad ( 38 )$$

subject to:

α^{T}D₀α=1 (39)

YₛBₛα≥\acute{0} (40)

The above formulation is a linearly constrained generalized eigenvalue problem. Since the constraints above are not convex, the problem cannot be solved using convex optimization methods. A solution can be found by using Sequential Quadratic Programming (SQP). SQP is an iterative method for constrained nonlinear optimization. Note that the iterative solver can get stuck in a local maxima.

### Constraint on Sink Elements 260

At least one stimulation element 260 configured as a sink (Vᵢ=0) must be present in the set of selected elements 260, such as a set of 8 elements 260. Setting a selected set of elements 260 to ground adds an equality constraint (Vᵢ=0) to the optimization formulation. The above optimization formulation is derived from the optimization formulation in terms of voltage vector V of equation (16). The equality constraint will set the selected components of the vector V to zero in the optimization formulation of equation (16). Thus the constraint Vᵢ=0 reduces the dimensionality of the optimization problem. The linearly constrained eigenvalue problem can be easily derived from equation (16) by setting the selected components of Vᵢ=0.

The linearly constrained eigenvalue problem is solved from 5566 different contact combinations. The solution corresponding to the maximum objective functional value in the 5566 combinations is chosen as the final solution. The corresponding eigenvector αₘₐₓ will represent the optimum value of the linear combiners. The corresponding element 260 combination will be the final element 260 configuration for optimum current steering.

The corresponding element 260 voltage values are given by:

\acute{V}ₘₐₓ=Bₛ60 ₘₐₓ (41)

The corresponding element 260 current values are given by

Íₘₐₓ=YₛBₛαₘₐₓ (42)

### Optimal Solution for Mixed Constrained Optimization

The floating stimulation element 260 formulation described in Section 6 uses the singular value decomposition for expressing the voltages as Bₛα and the corresponding currents as YₛBₛα. Bₛ provides a basis for voltage vector V such that it satisfies the floating element 260 constraint. Another way to select Bₛ is such that YₛBₛ is an identity matrix and Y_{f}Bₛ=0. Such a Bₛ can be found using Q-R decomposition of matrix [Yₛ^{T}, Y_{f}^{T}] and selecting first columns equal to number of sources. This changes the optimization problem to:

YₛBₛα≥\acute{0}

α≥\acute{0} (43)

Then the optimization problem can be defined as:

$${max}_{α} \text{:} {α}^{T} D α \qquad ( 44 )$$

which is subject to:

α^{T}D₀α=1 (45)

α≥\acute{0} (46)

To solve this constrained optimization problem, the following Lagrangian function can be considered:

L(α, λ, ω)=α^{T}Dₛα+λ(1−α^{T}D₀ₛα)+ω^{T}α (47)

where λ (≥0) and ω (≥0) are the Lagrangian multipliers for the equality and inequality constraints, respectively. The gradients of this Lagrangian function with respect to α, λ and ω are then equated to 0 to get the solution of the constrained optimization problem:

∇_{α}L=Dₛα−λD₀ₛα+ω=\acute{0} (48)

∇_{λ}L=1−α^{T}D₀ₛα=0 (49)

∇_{ω}L=α=\acute{0} (50)

Considering the Karush-Kuhn-Tucker (KKT) complementary slackness conditions:

ωᵢ=0, αᵢ≥0 (51)

ωᵢ≥0, αᵢ=0 (52)

which effectively correlates to:

ω^{T}α=0 (53)

Equation (48) can be rearranged as:

ω=λD₀ₛα−Dₛα=(λD₀ₛ−Dₛ)α (54)

A subset of indices P ⊆ {1,2,3, . . . n} is considered such that:

ω_{P}=\acute{0}, α_{P}≥\acute{0} (55)

ω_{\acute{P}}≥\acute{0}, α_{\acute{P}}=\acute{0} (56)

which can be represented as:

$$\{ \begin{matrix}{0}^{′} \\ {ω}_{{P}^{′}}\end{matrix} \} = \{ λ {D}_{0 s} - {D}_{s} \} \{ \begin{matrix}{α}_{P} \\ {0}^{′}\end{matrix} \} \qquad ( 57 )$$

This results in an equation (58) in the sub-matrix of λD₀ₛ−Dₛ:

λD₀ₛ_{_{PP}}α_{P}−Dₛ_{_{PP}}α_{P}=\acute{0}_{P} (58)

Equation (58) is a generalized eigenvalue problem. As eigenvector α obtained by solving the generalized eigenvalue problem also satisfies:

ω_{\acute{P}≥0} (59)

which is a solution for the optimization problem of equations (44) to (46). Solving the above generalized eigenvalue problem of equation (58) for each subset P that satisfies equation (59) results in a local maxima. Selecting the solution that has maximum eigenvalues among these local maxima gives the optimal solution for mixed constrained optimization. Since there are finitely many subsets P and there are finitely many eigenvalue solutions for each subset, the total number of local maxima is finite.

The solution of the optimization problem given in equations (44) to (46) gives the relative ratio of current values for different stimulation elements 260. It is possible that multiple combinations of stimulation elements 260 give the same solution of the optimization problem. In some embodiments, algorithm 15 compares such combinations and selects the combination with minimum number of active stimulation elements 260.

The optimization problem is defined such that the integration of current density in the complete domain Ω is unity. Hence, the solution of the optimization problem, to achieve optimum current steering, represents the integration of current density in the target region Ω_{T}. The total power provided by the stimulation elements 260 to achieve the solution of the optimization problem can be different for different target regions Ω_{T}.

In some embodiments, the solution of the optimization problem for all target regions Ω_{T} can be compared for a particular lead 265 configuration. As described hereabove in Section 3, any scale of the current values will satisfy the optimization problem. The scale for every location can be selected such that the total power in the target region Ω_{T} is the same for all locations of the target region Ω_{T} for a particular lead 265 configuration.

### Section 8—Global Optimization For All Focusing Locations

In Section 7, the optimization formulation that provides an optimal solution for a focusing location was described. Sourcing, ground and floating of stimulation elements 260 for each focusing location were independently selected for an optimal solution. Thus, it may happen that a completely different source, ground and floating configuration is chosen for adjacent focusing locations. It is important to minimize the change in configuration between adjacent focusing locations. This minimization will reduce the time involved in setting a configuration in hardware of apparatus 10, and it will improve the patient experience (e.g. via a reduced setup time or other reduced time in the clinic, and/or a smoother transition from one target location Ω_{T} to another).

In Section 7, for each focusing location an optimal solution can be chosen from 5566 possible configurations. A maximum objective function value for each configuration at a focusing location is provided. In some embodiments, a configuration is selected such that it maximizes the objective function value and minimizes changes in sourcing and grounding stimulation elements 260 between adjacent focusing locations. This configuration provides a global optimization problem across all focusing locations.

### Section 9—Optimization Considering Unusable Stimulation Elements 260

As described hereabove in Section 7, it was shown that 5566 electrode combinations are possible when considering 8 active stimulation elements 260, 4 each on two leads 265, out of the possible 16 elements 260 (i.e. where each lead 265 comprises 8 elements 260). These combinations were obtained based on the constraints of a maximum of 4 sourcing elements 260 and a minimum of 1 grounded element 260. The set of active elements 260 will change based on the lead 265 configuration and the focus location. For the selected set of active elements 260, the optimum solution out of the 5566 combinations can be selected. However, it is possible that some of the elements 260 are not usable (e.g. due to the contacts of the elements 260 being shorted, a broken wire or other conductor, and the like).

These unusable stimulation elements 260 can be considered in the optimization problem by neglecting those element 260 combinations out of the 5566 combinations, which use any of the unusable elements 260 either as a sourcing or ground electrode. In some embodiments, algorithm 15 can determine the optimum element 260 combination out of the remaining combinations (e.g. in an efficient way due to the elimination of unusable elements 260).

### Section 10—Methods to Specify Lead 265 Configurations

In some embodiments, algorithm 15 is configured to determine a stimulation paradigm SP for a given implanted geometry and implanted position of leads 265. The implanted position of one or more leads 265 can be specified relative to the patient's body and/or relative to one or more other leads 265 (e.g. a lead 265 used as a reference or otherwise). In some embodiments, algorithm 15, in determining a stimulation paradigm SP, assumes that one or more leads 265 are in a relatively straight geometry when implanted, and/or two or more leads are in a relatively straight and relatively parallel geometry when implanted. In some embodiments, algorithm 15 can assume that two or more leads 265 are implanted in a staggered geometry. Lead 265 implantation geometry can be determined by algorithm 15 using one or more different methods (e.g. based on impedance measurements, image analysis, and/or operator input). The determined implantation geometry of lead 265 can be then used by algorithm 15 in determining a stimulation paradigm SP.

In some embodiments, algorithm 15 assumes two leads 265 are straight and parallel to each other and have a certain inter-lead distance.

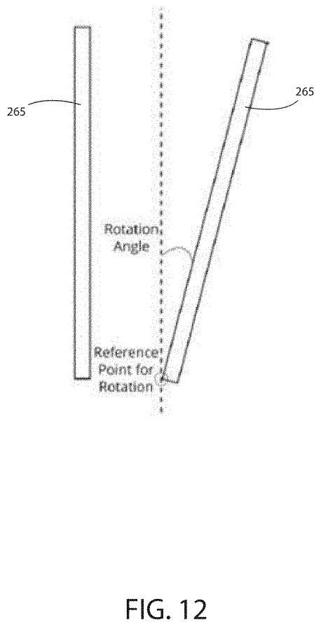

In some embodiments, algorithm 15 assumes two leads 265 are straight, but a first lead 265 is assumed to be at an angle with respect to (i.e. not parallel with) the second lead 265. The position and orientation of the second lead 265 can be defined by specifying a reference point and the rotation angle about the reference point, as shown in FIG. 12.

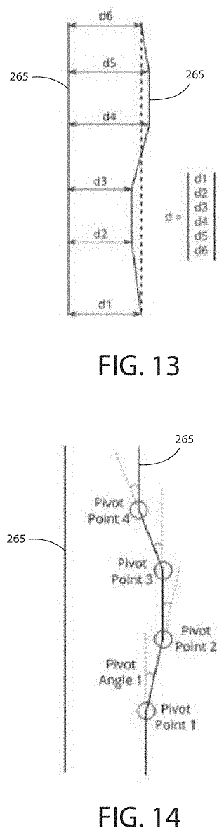

In some embodiments, algorithm 15 assumes a first lead 265 is straight, and a second lead 265 is defined by specifying the distance of different points along the axis of the second lead 265 from the first lead 265. Thus the inter-lead distance will be a set of values, rather than a single value, as shown in FIG. 13.

In some embodiments, algorithm 15 assumes a first lead 265 is straight, and a second lead 265 is defined in terms of a spline function. Thus, the second lead 265 will have a smooth curved profile.

In some embodiments, algorithm 15 assumes a first lead 265 is straight, and a second lead 265 is assigned a number of pivot points. The geometry of the second lead 265 can be defined by specifying the location of the pivot points and the rotation angle about the pivot points, as shown in FIG. 14.

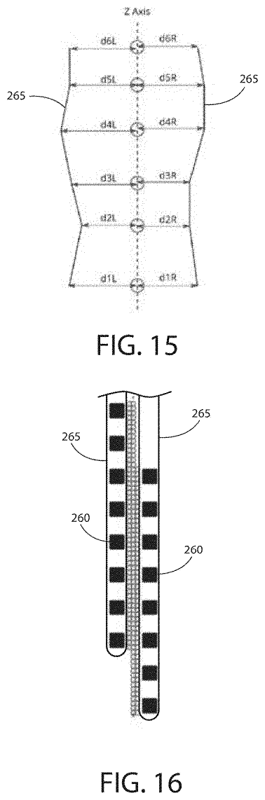

In some embodiments, algorithm 15 assumes both a first and second lead 265 are implanted in a non-straight geometry. Algorithm 15 can use a polar coordinate system, with the Z-axis along the spinal canal of the patient, and the polar plane along the transverse plane of the patient's body, as shown in FIG. 15.

In some embodiments, algorithm 15 can assume two leads 265 are lying in the same plane, where the location of each stimulation element 260 can be defined by its reference point along the Z-axis and the distance of the element 260 center from this reference point in that plane. After specifying all the element 260 locations, algorithm 15 can define a spline function for each lead 265.

In some embodiments, algorithm 15 can assume two leads 265 are non-straight, and bend across different planes. The location of each stimulation element 260 can be defined by its reference point along the Z-axis and the angle and distance from the Z-axis. After specifying all the element 260 locations, algorithm 15 can define a spline function for each lead 265.

In some embodiments, a single lead 265 with 4 or 8 stimulation elements 260 can be assumed by algorithm 15, and all of the elements 260 on the lead 265 can be considered for optimization. The lead 265 can be assumed to be straight or non-straight. For straight leads 265, the lead 265 location can be specified in terms of the center coordinates of an element 260 used as a reference, and the rotation angle between the lead 265 axis and the spinal canal axis of the patient. For non-straight leads, algorithm 15 can define location of the lead 265 by one or more of the methods described hereabove.

In some embodiments, one lead 265 can comprise a paddle lead (e.g. when apparatus 10 further comprises one or more additional leads 265 which comprise leads with a cylindrical geometry). The stimulation elements 260 of the paddle lead-based lead 265 can be arranged in one or more desired patterns. An advantage to this configuration is that the position of the elements 260 of a lead 265 comprising a paddle lead are unlikely to change over time (e.g. less likely to migrate versus a cylindrical geometry lead). Algorithm 15 can utilize the formulation and optimization solutions described hereabove in Sections 3 and 4 with a lead 265 comprising a paddle lead.

### Section 11—Methods to Specify Focus Locations

In some embodiments, apparatus 10 includes a user interface that allows the patient and/or other operator of apparatus 10 to specify a location into which current delivered by stimulation elements 260 can be delivered (e.g. steered). Focus locations can be modeled in terms of a cylindrical target region Ω_{T} in the spinal canal of the patient. The location of this target region Ω_{T} can be varied along the height and width of the spinal canal. The length of the canal can be kept such that the target region Ω_{T} is contained within the spinal canal. The target regions Ω_{T} can be defined as a grid of 3 columns and multiple rows. The vertical distance between focus locations can be set to 1 mm, and the horizontal distance between the focus locations can be set to 0.5 mm. The focus locations near each lead 265 can be defined so that the area in front of each lead 265 is covered. For central focus locations (equidistant from two leads 265), the combined area in front of both leads 265 can effectively receive stimulation energy from the stimulation elements 260. FIG. 16 shows a representative image (with distances not to scale) of the focus locations considered for a staggered lead 265 implantation arrangement. The solid boxes represent the elements 260 of two leads 265, and the circles represent the focus locations. The regions present only in front of a single lead 265 are not considered as it is difficult to focus the current delivered by elements 260 in those regions.

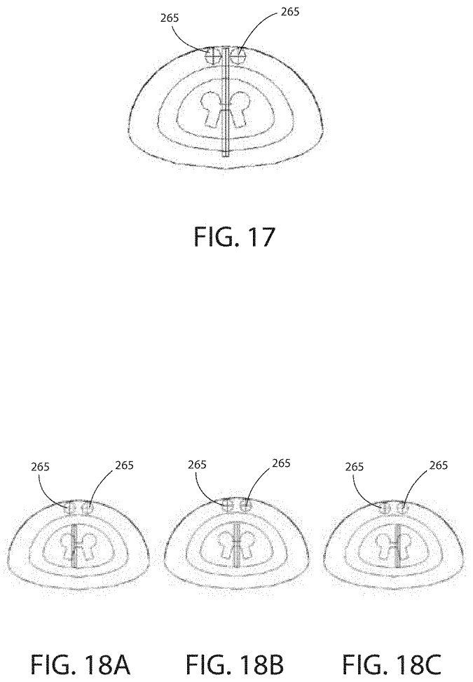

In some embodiments, the length of a cylindrical target region Ω_{T} can be modified such that the target region Ω_{T} is contained within the grey matter and white matter of the spinal cord only. FIG. 17 shows a cross-sectional view of a geometry constructed with a target region Ω_{T} constrained within the spinal canal. FIGS. 18A, 18B, and 18C show three cross-sectional views of a geometry constructed with the target region Ω_{T} constrained within the grey matter and the white matter of the spinal canal. For the target region Ω_{T} that is defined to be within the grey matter and the white matter, the locations of the target regions Ω_{T} in the grid of the 3 columns of the grey matter and white matter of the spinal canal can be kept constant. This arrangement enables having the same target grid for different lead 265 configurations. Since the target regions do not change with respect to lead 265 configurations, the objective function values (obtained as an output from algorithm 15) can then be compared. This approach helps in analyzing which lead 265 configuration(s) can focus better on a given target region Ω_{T}.

### Section 12—Data Handling

The optimization problem of algorithm 15 requires input of the C, C₀ and Y matrices, as described hereabove in Section 3. These matrices can be obtained from the FEA-based simulations described herein. The C₀ and Y matrices are dependent on the lead 265 configuration and every focus location has its corresponding C matrix. As described hereabove in Section 11, the focus locations (number and location) can be determined based on the lead 265 configuration. Via running simulations for the different focus locations, the C, C₀ and Y matrices can be predetermined and stored in library 16 for different lead 265 configurations. Algorithm 15 can determine the optimum stimulation element 260 activation patterns (e.g. potential stimulation paradigm SP parameters) for different focus locations. This activation data can also be predetermined and stored in library 16 for different lead 265 configurations, such as when algorithm 15 determines stimulation paradigm SP based on a correlation of this predetermined information (e.g. a correlation based on implant locations and/or implant geometries of two or more leads 265).

In some embodiments, these data sets of matrices and optimum stimulation element 260 activation patterns (e.g. stimulation paradigms SP) can be stored as text and/or data files in a remote device such as a computer (e.g. where at least a portion of library 16 is located in the remote device). This remote device can be in communication (e.g. wireless communication) with programmer 600, an external device 500, and/or other component of apparatus 10 that includes a user interface for accessing the relevant stimulation element 260 activation patterns.

In some embodiments, these data sets of matrices and optimum stimulation element 260 activations patterns (e.g. stimulation paradigms SP) can be stored in a database system (e.g. a library 16 comprising a database), that is included and/or otherwise available to programmer 600, an external device 500, and/or other component of apparatus 10 that includes a user interface for accessing the relevant stimulation element 260 activation patterns.

The solution of the optimization problem by algorithm 15 defines the stimulation paradigm SP, which includes the values for various currents to be delivered by the selected stimulation elements 260. As any scale of currents delivered will also satisfy this optimization problem, the relative ratio of the current values is important, rather than the absolute magnitudes of the electrode current. Hence, the current values are stored (e.g. in library 16) after normalization. Normalization can be done separately for each combination of elements 260, and for each location of the target region Ω_{T}, such as when all current values are integer values between 0 and 255. Elements 260 that are floating are assigned a value of 0. The largest sourcing current amplitude is assigned a value of 255.

In some embodiments, normalization is performed for a particular lead 265 configuration so as to achieve the same power through the target region Ω_{T} for any location of the target region Ω_{T}. In these embodiments, the largest sourcing current amplitude may not have a value of 255 for all locations.

In some embodiments, Y, C, and C₀ matrices are stored (e.g. in library 16) and used for computations required for operating a user interface of apparatus 10 (e.g. a user interface of a programmer 600 and/or external device 500), when apparatus 10 is operating in a “manual mode”, such as is described herebelow in reference to section 14.

### Section 13—User Interface

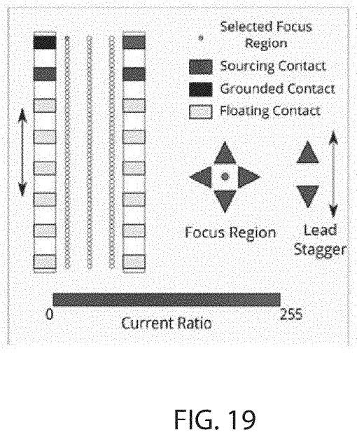

In some embodiments, apparatus 10 comprises a user interface (e.g. user interface 680 of programmer 600 and/or user interface 580 of external device 500) that is configured to provide to an operator (e.g. a clinician of the patient) an interface to select parameters of a stimulation paradigm SP, such as to select a vertical stagger of two or more leads 265. For a selected stagger value, the operator can then select the location of the target region Ω_{T}. For the selected target region Ω_{T}, the activation of stimulation elements 260 can be represented through a color scale, darkness scale, and/or other visualizable differentiation. The relative ratio of current values, such as a ratio normalized to a range of integer values (e.g. between 0 and 255), can be used. FIG. 19 illustrates a typical representation of the user interface used by an operator to enter one or more parameters of a stimulation parameter SP.

### Section 14—User Interface Manual Mode (Inverse Problem)

In some embodiments, a user interface of apparatus 10 (e.g. user interface 680 of programmer 600 and/or user interface 580 of external device 500) is configured to provide a “manual mode” of operation for one or more operators of apparatus 10. In the manual mode, the patient or other operator of apparatus 10 can specify the stimulation paradigm SP to be used (e.g. a stimulation paradigm SP that defines current sourcing, grounding and floating states of the stimulation elements 260). Algorithm 15 can be configured to predict a location on a grid of target locations Ω_{T}, such that current steering defined by the stimulation paradigm SP is at a maximum. In these manual mode configurations, library 16 can include various forms and types of data.

The C, C₀ and Y matrices (e.g. predetermined matrices) can be processed along with the values of the current for stimulation elements 260 (e.g. inputted current values) to predict the location on the grid of target locations Ω_{T}. In some embodiments, if only sourcing and sinking current magnitudes are specified by an operator of apparatus 10, and given the current values 1, the voltage values V can be calculated from the conductance matrix, for the specified geometry and location of leads, using the following linear relation:

Í=Y\acute{V} (60)

As the conductance matrix Y is not invertible, equation (60) can be used to find the best matching values for voltage V for the current values I.

Using the C and C₀ matrices, the ratio of integration of square of current density in the target location Ω_{T} with respect to the complete domain Ω, can be calculated as follows:

$$\frac{{{V}^{′}}^{T} C {V}^{′}}{{{V}^{′}}^{T} {C}_{0} {V}^{′}} \qquad ( 61 )$$

Different locations of the target region Ω_{T} can have different C matrices. The ratio in equation (61) can be evaluated at all locations. In some embodiments, multiple circles in a grid of target locations Ω_{T} (as shown in FIG. 19) are assigned a visualizable identifier (e.g. color, shade, and/or hue) that is proportional to the evaluated ratio (equation 61) at that location along with a confidence interval at that location. A visual representation of the distribution of the evaluated ratio (equation 61) enables an operator to modify the values of the currents of the stimulation elements 260 that are sourcing and/or sinking currents. In some embodiments, the location with the maximum ratio (equation 61) value can be selected and the corresponding circle in the grid of target regions (e.g. as shown in FIG. 19) can be highlighted on the user interface.

As described herein, apparatus 10 via algorithm 15 can be configured to steer current to one or more target locations Ω_{T}, such as when the stimulation paradigm SP produced by algorithm 15 is determined and/or otherwise is based on FEA techniques (e.g. FEA techniques used to focus the electromagnetic fields generated by delivery of the determined currents by the selected stimulation elements 260). In some embodiments, algorithm 15 utilizes one or more constraints in order to determine stimulation paradigm SP, such as by limiting the number of stimulation elements 260 configured as a source and/or a sink (e.g. algorithm 15 comprises an optimization problem with these constraints). In some embodiments, algorithm 15 determines the stimulation paradigm SP based on one or more stimulation elements 260 being classified as being “open” and/or “shorted” (e.g. due to broken conductors, shorted conductors, and/or other potential issues with lead 265 and/or another component of delivery device 200). In some embodiments, algorithm 15 determines a stimulation paradigm SP based on a library of pre-determined information (e.g. as stored in library 16). In these embodiments, implantation locations and geometries of one, two, or more leads 265 can be identified (e.g. lead 265 linear and/or curvilinear implantation shape, spacing and/or orientation between two leads 265, and other implantation information entered into apparatus 10), and used to find a correlation to the library of pre-determined information. This pre-determined information is generated by specifying locations and/or geometries of one, two or more leads 265 in FEA simulations and obtaining the stimulation paradigm SP using algorithm 15.

In some embodiments, algorithm 15 is configured to allow an operator of apparatus 10 to specify a lead configuration. Specifying a lead configuration includes defining implanted location and implanted geometry of one, two or more leads 265 and their associated stimulation elements 260 with respect to the patient's body and/or relative to one or more other leads 265 and their associated stimulation elements 260.

In some embodiments, algorithm 15 is configured to allow an operator to specify one or more target locations Ω_{T} to receive stimulation energy delivered by stimulation elements 260, such as via a user interface (e.g. user interface 680 of programmer 600 and/or user interface 580 of an external device 500), as described herein. In some embodiments, the user interface allows selection of one or more target locations Ω_{T} from a predefined grid of target locations Ω_{T}. Based on the selected target location(s) Ω_{T}, a precomputed stimulation paradigm SP (i.e. precomputed set of stimulation parameters) is selected for stimulating the selected target location(s) Ω_{T}. A stimulation paradigm SP specifies current sourcing, grounding and floating states of the stimulation elements 260, and can also specify the magnitudes of currents to be delivered on each element 260. The selected stimulation paradigm SP can be displayed on the user interface, such as using color coding of icons on the user interface that represent the implanted stimulation elements 260.

In some embodiments, algorithm 15 is configured to produce a stimulation paradigm SP using an inverse solution, such as an inverse solution that predicts an anatomical location to be stimulated based on a given stimulation paradigm SP (e.g. given a set of stimulation elements 260 to be configured as anodes and/or cathodes, as well as a set of current amplitudes to be delivered to each), as described herein.

As described herein, apparatus 10 can comprise one or more implantable devices, each of which can include, one, two, or more leads 265. Each lead 265 can comprise one, two, or more stimulation elements 260, such as a set of stimulation elements 260 each comprising an electrode that can be configured in one of at least two configuration states, such as a first configuration state in which the stimulation element 260 is a source and/or sink of current that stimulates tissue, and a second configuration state in which the stimulation element 260 is electrically passive (e.g. in a “floating” state in which current is neither sourced nor sinked). In some embodiments, a stimulation paradigm SP produced by algorithm 15 defines a set of one, two or more elements 260 as being configured as a current source, a current sink, or both. In some embodiments, a stimulation paradigm SP defines each element 260 of a delivery device 200 to be configured to be one, two, or all three of: a current source, a current sink, and/or floating (e.g. unconnected or otherwise electrically passive). In some embodiments, a stimulation paradigm SP defines a first set of one or more elements 260 to be configured to source and/or sink current, and a second set of elements 260 to be configured in a floating state. Algorithm 15 can be configured to create a set of multiple stimulation paradigms SP to correspondingly stimulate (e.g. optimally stimulate) a set of one, two, or more target locations QT.

In some embodiments, algorithm 15 is configured to create a stimulation paradigm SP in which, for each lead 265, the 4 stimulation elements 260 that are most proximate the target location Ω_{T} are configured to source and/or sink current, and all of the remaining elements 260 of each lead 265 are configured in a floating state.

In some embodiments, apparatus 10 is configured to allow a set of one or more stimulation elements 260 to be in a first arrangement in which a particular current is delivered by the element 260, and/or a set of one or more stimulation elements 260 to be in a second arrangement in which the element 260 is set to a particular voltage. For example, one or more stimulation elements 260 can be configured to deliver a positive or zero current, while one or more other stimulation elements 260 can be set to a ground voltage (e.g. grounded at zero voltage). Alternatively or additionally, one or more stimulation elements 260 can be configured to deliver a negative or zero current, while one or more other stimulation elements 260 are set to a ground voltage (e.g. grounded at zero voltage). In some embodiments, no more than 4 stimulation elements 260 are set to deliver positive and/or negative current, and the remaining stimulation elements 260 are set to a ground voltage and/or are configured in a floating state.

In some embodiments, apparatus 10 (e.g. algorithm 15) is configured to determine a stimulation paradigm SP that preferentially stimulates a target region Ω_{T} by maximizing the ratio of the integral of the square of the current density in the target region Ω_{T} to the integral of the square of the current density of a large part of the patient's body, Ω_{LARGE}. For example, Ω_{T} can comprise a segment of the spinal canal, and Ω_{LARGE} can comprise the entire spinal canal, or Ω_{T} can comprise an epidural fat region of the spinal canal, and Ω_{LARGE} can comprise the spinal canal, vertebral column, and surround muscles. In some embodiments, Ω_{LARGE} comprises the entire body of the patient. In some embodiments, apparatus 10 calculates (e.g. via algorithm 15) a stimulation paradigm SP by solving one or more generalized eigenvalue problems, such as to determine a stimulation paradigm that determines optimized state configurations (e.g. source, sink, or floating states) of stimulation elements 260 as well as optimized currents to be delivered by one or more of those elements 260 and/or voltages to be set for one or more of those elements 260.

In some embodiments, algorithm 15 produces one or more stimulation paradigms SP that configures a first set S1 of stimulation elements 260 to source and/or sink current, such as a set of elements 260 (e.g. 4, 6, or 8 elements 260) that are most proximate a target location Ω_{T}, while a second set S2 of elements 260 comprising the remaining elements 260 (e.g. the elements 260 of the one or more implanted leads 265 not in the first set) are configured in a floating state. In some of these embodiments, stimulation energy delivered by the first subset S1_{A} of the first set S1 of elements 260 can be current-controlled (e.g. a current input by an operator and/or determined by algorithm 15), and a second subset S1B of the first set S1 of elements 260 can be voltage-controlled (e.g. a voltage input by an operator and/or determined by algorithm 15). The first subset S1_{A} of elements 260 can be set to a positive and/or zero current, and the second subset S1_{B} of elements 260 can be set to a ground voltage. Alternatively or additionally, the first subset S1_{A} can be set to a negative and/or zero current, and the second subset S1_{B} can be set to a ground voltage. In some embodiments, the first subset S1_{A} comprises no more than 4 elements 260.

In some embodiments, algorithm 15 produces a stimulation paradigm SP based on maximizing the integral of the square of the current density in the target region Ω_{T} to the integral of the square of the current density in a larger volume of the patient's body, as described herein. For example, algorithm 15 can calculate one or more optimized stimulation paradigms SP by solving one or more generalized eigenvalue problems, for example via an unconstrained optimization problem or optimization problem with floating contacts or mixed formulation, such as are described in Sections 4, 6, and/or 7.

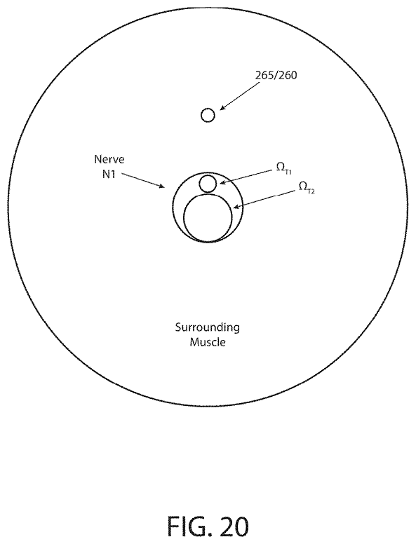

Referring now to FIG. 20, an anatomical sectional view of a lead implanted proximate a peripheral nerve is illustrated, consistent with the present inventive concepts. Apparatus 10 can be configured to deliver stimulation energy to the peripheral nervous system, such as when delivering stimulation energy to one or more nerves outside of the brain and spinal cord to treat pain of the peripheral nervous system (e.g. chronic pain). For example, one, two or more leads 265 (e.g. portions of one or more leads 265, each containing one or more stimulation elements 260), can be positioned proximate one or more peripheral nerves, such as nerve N1 shown. In these peripheral nerve stimulation (PNS) embodiments, apparatus 10 can be configured to steer current delivered by the stimulation elements 260 (e.g. electrode-based stimulation elements 260), such as is described herein.

Apparatus 10 can provide stimulation energy defined by a stimulation paradigm SP (e.g. an excitation pattern determined by algorithm 15) such as to steer current delivered by the stimulation elements 260 toward one or more peripheral nerves to be stimulated. The stimulation paradigm SP can define an optimized current delivery profile for one or more stimulation elements 260, as described herein. The stimulation paradigm SP can define one or more stimulation elements to be floating (e.g. continuously and/or intermittently), and/or configured as a ground (e.g. continuously and/or intermittently), also as described herein.

Lead 265 of FIG. 20 can include 4 stimulation elements 260 (e.g. electrode-based stimulation elements) and lead 265 can comprise a diameter of 1.3 mm. Nerve N1, shown proximate lead 265, can comprise a diameter of 7 mm. Lead 265 has been implanted in an orientation relatively parallel to the axis of nerve N1 (e.g. such that each stimulation element 260 is relatively the same orthogonal distance from nerve N1). Algorithm 15 can perform current steering calculations based on the assumption that the nerve N1 has a homogeneous structure. Nerve N1 can comprise two target regions, Ω_{T1} and Ω_{T2} shown. Target region Ω_{T1} is positioned proximate a side of lead 265 as shown. Target region Ω_{T2} is a larger volume than target region Ω_{T1} and is positioned farther away from lead 265 than target region Ω_{T1}, as shown. Target region Ω_{T1} is approximately one-third the diameter of nerve N1, and target region Ω_{T2} is approximately two-thirds of the diameter of nerve N1. The two target regions are shown in FIG. 20 and described herein to provide an example of how algorithm 15 can steer current in regions relatively near to lead 265 (e.g. target region Ω_{T1}) and/or relatively distant from lead 265 (e.g. target region Ω_{T2}). Algorithm 15 can perform current steering calculations based on the assumption that nerve N1 is surrounded by muscle tissue (e.g. relatively homogenous muscle tissue).

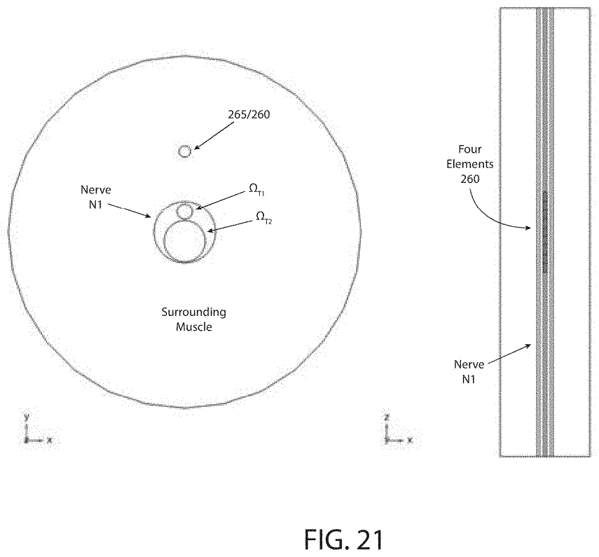

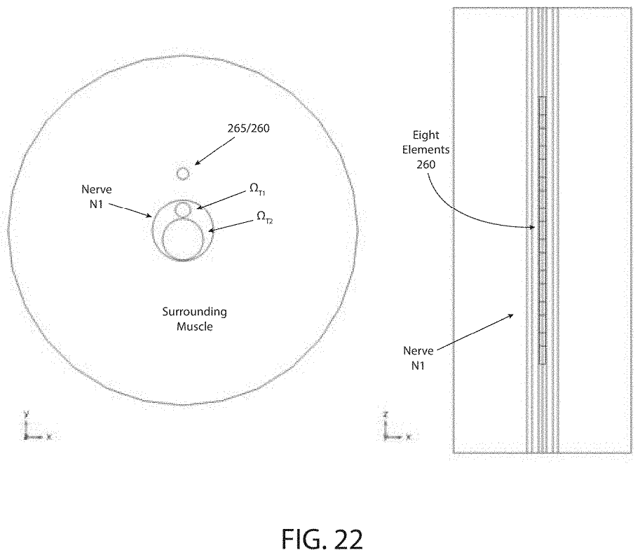

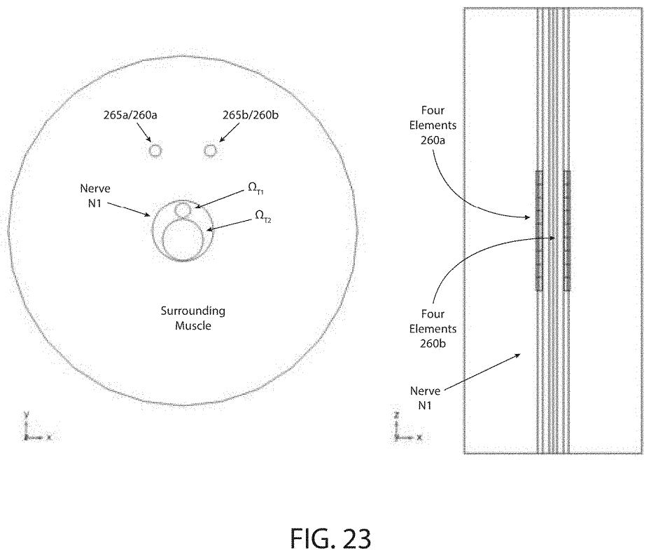

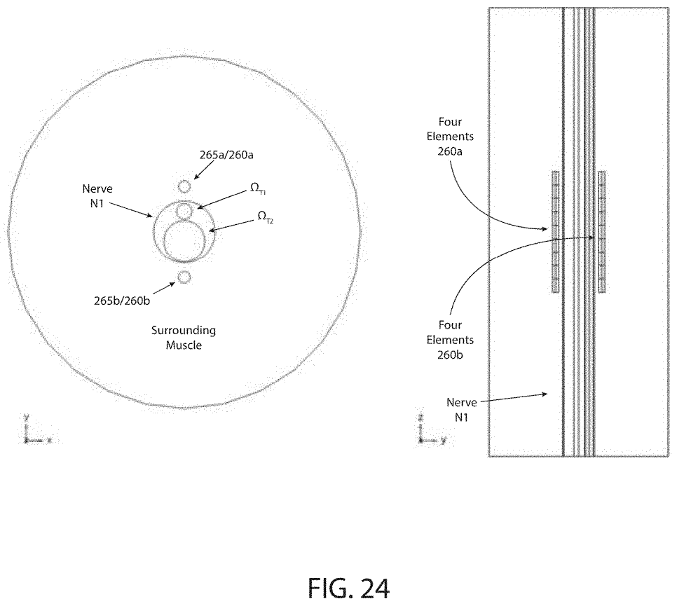

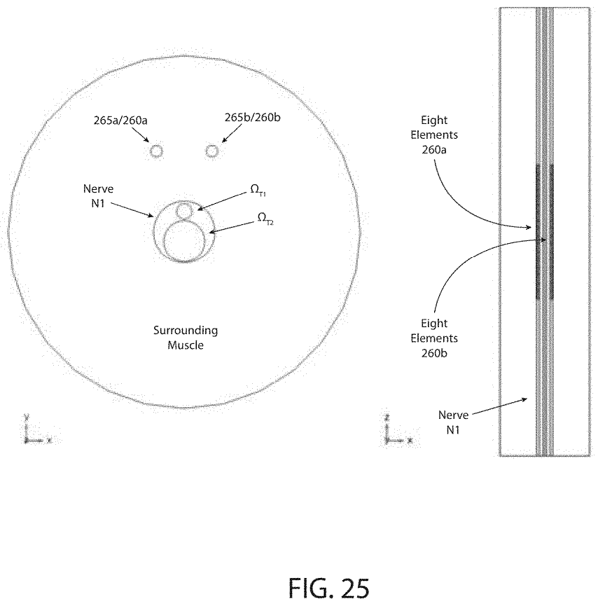

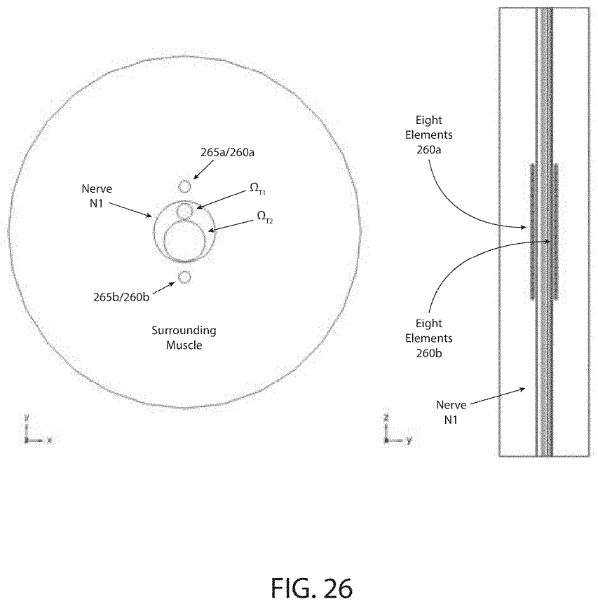

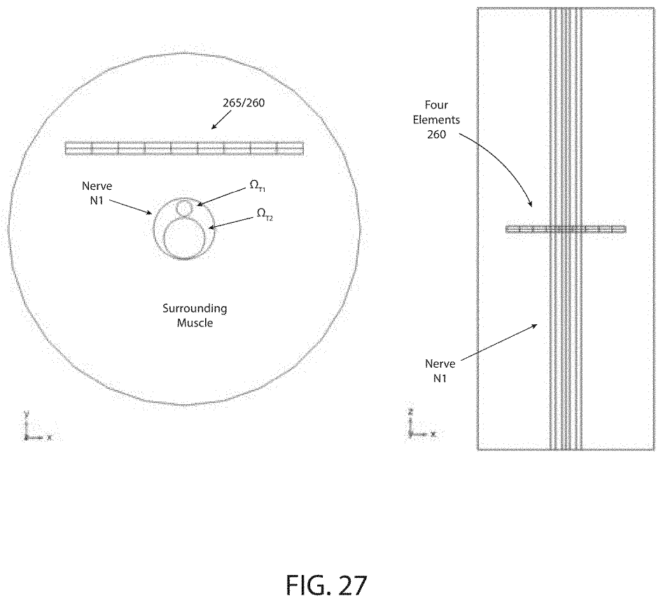

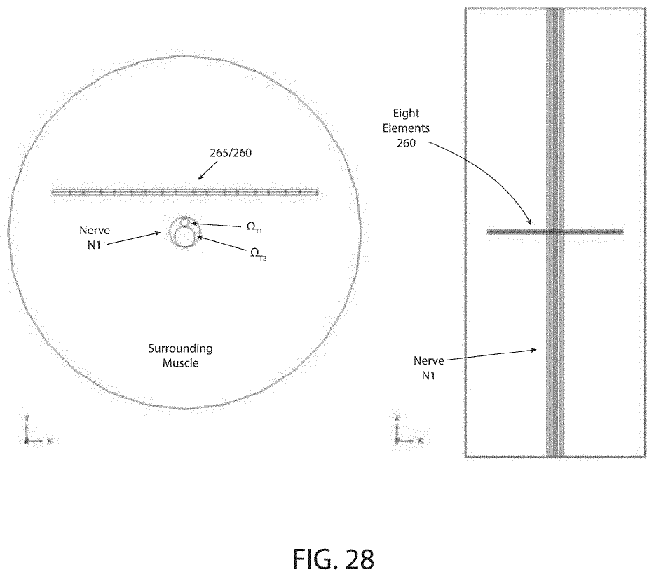

Each lead 265 of a delivery device 200 can comprise different configurations (e.g. different quantities and/or positioning of stimulation elements 260), and each lead 265 can be implanted relatively close to one or more nerves in various arrangements (e.g. same or different sides of a nerve). For example, eight different lead 265 design and implantation configurations can include: (1) a single lead 265 comprising four stimulation elements 260, the lead 265 implanted relatively parallel to the axis of a nerve to be stimulated (as shown in FIG. 21); (2) a single lead 265 comprising eight stimulation elements 260, the lead 265 implanted relatively parallel to the axis of a nerve to be stimulated (as shown in FIG. 22); (3) two leads 265, each comprising four stimulation elements 260, each lead 265 implanted parallel to the axis of a nerve to be stimulated and positioned on the same side of that nerve (as shown in FIG. 23); (4) two leads 265, each comprising four stimulation elements 260, the two leads 265 implanted parallel to the axis of a nerve to be stimulated and positioned on opposite sides of that nerve (as shown in FIG. 24); (5) two leads 265, each with eight stimulation elements 260, each lead 265 implanted parallel to the axis of a nerve to be stimulated and positioned on the same side of that nerve (as shown in FIG. 25); (6) two leads 265, each with eight stimulation elements 260, the two leads 265 implanted parallel to the axis of a nerve to be stimulated and positioned on opposite sides of that nerve (as shown in FIG. 26); (7) a single lead 265 comprising four stimulation elements 260 and implanted perpendicular to a nerve to be stimulated (as shown in FIGS. 27); and (8) a single lead 265 comprising eight stimulation elements 260 and implanted perpendicular to a nerve to be stimulated (as shown in FIG. 28).

In determining an algorithm 15 for current steering in single or multiple lead 265 configurations, multiple orthogonal distances between elements 260 and the central axis of nerve N1 can be considered, such as distances of 1 mm, 3 mm, and/or 5 mm. In determining an algorithm 15 for current steering in multiple leads 265 configurations, staggering of the implantation locations of stimulation elements 260 can be considered.

### Section 15—PNS Formulation

As described herein, apparatus 10 can comprise an algorithm 15 that uses a formulation to steer current delivered by electrode-based stimulation elements 260 into one or more target regions, such as target region Ω_{T}, of a tissue domain Ω. The tissue domain Ω can be part of the patient's peripheral nervous system. In some embodiments, apparatus 10 is configured to stimulate one or more peripheral nerves within a peripheral nerve bundle (e.g. to treat pain), such as by delivering current from one or more stimulation elements 260 of one or more leads 265 that have been positioned proximate the nerve bundle. The nerve bundle can comprise multiple fascicles. Each of the multiple fascicles can comprise various densities (e.g. packing densities of nerve fibers) and can comprise nerve fibers of various types (e.g. Aα, Aβ, c, and/or Aδ nerve fibers). In some embodiments, apparatus 10 is configured to steer and/or otherwise bias the delivered current toward one or more target fascicles (e.g. fascicles comprising densely packed Aα or Aβ nerve fibers), and/or to steer and/or otherwise bias current away from one or more non-target fascicles (e.g. fascicles comprising Aδ nerve fibers). For example, algorithm 15 can be configured to determine stimulation parameters to steer current delivered by the one or more stimulation elements 260 (e.g. electrodes) toward one or more target fascicles. In some embodiments, algorithm 15 can determine a stimulation paradigm SP (e.g. stimulation parameters) during a trialing procedure (e.g. a procedure in which patient feedback regarding pain relief and/or paresthesia is determined). Alternatively or additionally, algorithm 15 can analyze image data (e.g. image data representing a nerve bundle, surrounding tissue, and/or the one or more leads 265 proximate the nerve bundle), such as to determine stimulation parameters required to steer the current delivered by stimulation elements 260 toward the target fascicles (e.g. and away from non-target tissue).

The voltage (U) distribution in the domain Ω (e.g. including at least tissue of the patient's peripheral nervous system) is defined by:

∇·σ∇U=0inΩ

Letting fₓᵢ be the voltage field defined over the domain such that voltage for i^{th} stimulation element 260 is set to 1 (vᵢ=1) and all other stimulation elements 260 are set to circuit ground (also referred to as simply “ground” herein). Hence, any voltage distribution in the domain Ω can be represented as a linear combination of voltage field fₓᵢ, where:

f_{\acute{x}}=Σᵢvᵢf_{\acute{x}i}

The corresponding current density is given by:

σ∇f_{\acute{x}}=σΣᵢvᵢ∇f_{\acute{x}i}

where σ is the conductivity tensor defined over the entire domain Ω. In the other embodiments, the conductivity tensor comprises a diagonal form:

$$σ = [ \begin{matrix}{σ}^{1} 00 ; & 0 {σ}^{2} 0 ; & 00 {σ}^{3}\end{matrix} ] = ( \begin{matrix}{σ}^{1} & 0 & 0 \\ 0 & {σ}^{2} & 0 \\ 0 & 0 & {σ}^{3}\end{matrix} ) .$$

In some embodiments, the conductivity tensor σ comprises a symmetric positive definite matrix. The following formulation is described using a diagonal form.
It can be desirable to maximize the ratio of integration of square of current density in the target region Ω_{T} with respect to the integration of square of current density in the entire domain Ω. The optimization problem can be defined as:

$${max}_{{v}_{i}} \text{:} \frac{\int \int {\int }_{{Ω}_{T}} {‖ σ ∇ {f}_{{x}^{′}} ‖}^{2}}{\int \int {\int }_{Ω} {‖ σ ∇ {f}_{{x}^{′}} ‖}^{2}}$$

$${max}_{{v}_{i}} \text{:} \frac{\int \int {\int }_{{Ω}_{T}} {‖ σ {\sum }_{i} {v}_{i} ∇ {f}_{{x}^{′} i} ‖}^{2}}{\int \int {\int }_{Ω} {‖ σ {\sum }_{i} {v}_{i} ∇ {f}_{{x}^{′} i} ‖}^{2}}$$

The ratio will increase either by increasing the numerator or by decreasing the denominator. The denominator represents the sum of two terms: integration of the square of current density in the target region Ω_{T}, and integration of square of current density in the remaining domain. Thus, defining the optimization problem in terms of this ratio provides an advantage of maximizing the integration of square of current density in the target region Ω_{T} and simultaneously minimizing the integration of square of current density in the remaining domain.
The components of ∇f_{\acute{x}i} can be considered as (g_{\acute{x}i}¹g_{\acute{x}i}²g_{\acute{x}i}³)^{T}. The optimization problem can then be expressed as:

$${max}_{{v}_{i}} \text{:} \frac{\int \int {\int }_{{Ω}_{T}} {\sum }_{j} {( {σ}^{j} {\sum }_{{i}^{V} i} {g}_{{x}^{′} i}^{j} )}^{2}}{\int \int {\int }_{Ω} {\sum }_{j} {( {σ}^{j} {\sum }_{i} {v}_{i} {g}_{{x}^{′} i}^{j} )}^{2}}$$

Expanding the term Σⱼ(σ^{j}Σᵢvᵢg_{\acute{x}i}^{j})², the following results,

$${\sum }_{j} {( {σ}^{j} {\sum }_{i} {v}_{i} {g}_{{x}^{′} i}^{j} )}^{2} = {\sum }_{i} {v}_{i}^{2} ( {\sum }_{j} {( {σ}^{j} )}^{2} {( {g}_{{x}^{′} i}^{j} )}^{2} ) + {\sum }_{i} {\sum }_{k \neq i} {v}_{i} {v}_{k} ( {\sum }_{j} {( {σ}^{j} )}^{2} {g}_{{x}^{′} i}^{j} {g}_{{x}^{′} k}^{j} )$$

Substituting in our optimization problem, the following results,

$$\begin{matrix}{max}_{{v}_{i}} \text{:} \frac{\begin{matrix}\int \int {\int }_{{Ω}_{T}} [ {\sum }_{i} {v}_{i}^{2} ( {\sum }_{j} {( {σ}^{j} )}^{2} {( {g}_{{x}^{′} i}^{j} )}^{2} ) + \\ {\sum }_{i} {\sum }_{k \neq {{i}^{{V}_{i}}}^{{V}_{k}}} ( {\sum }_{j} {( {σ}^{j} )}^{2} {g}_{{x}^{′} i}^{j} {g}_{{x}^{′} k}^{j} ) ]\end{matrix}}{\begin{matrix}\int \int {\int }_{Ω} [ {\sum }_{i} {v}_{i}^{2} ( {\sum }_{j} {( {σ}^{j} )}^{2} {( {g}_{{x}^{′} i}^{j} )}^{2} ) + \\ {\sum }_{i} {\sum }_{k \neq i} {v}_{i} {v}_{k} ( {\sum }_{j} {( {σ}^{j} )}^{2} {g}_{{x}^{′} i}^{j} {g}_{{x}^{′} k}^{j} ]\end{matrix}} & ( A1 ) \\ {max}_{{v}_{i}} \text{:} \frac{\begin{matrix}{\sum }_{i} {v}_{i}^{2} [ \int \int {\int }_{{Ω}_{T}} {\sum }_{j} {( {σ}^{j} )}^{2} {( {g}_{{x}^{′} i}^{j} )}^{2} ] + \\ {\sum }_{i} {\sum }_{k \neq i} {v}_{i} {v}_{k} [ \int \int {\int }_{{Ω}_{T}} {\sum }_{j} {( {σ}^{j} )}^{2} {g}_{{x}^{′} i}^{j} {g}_{{x}^{′} k}^{j} ]\end{matrix}}{\begin{matrix}{\sum }_{i} {v}_{i}^{2} [ \int \int {\int }_{Ω} {\sum }_{j} {( {σ}^{j} )}^{2} {( {g}_{{x}^{′} i}^{j} )}^{2} ] + \\ {\sum }_{i} {\sum }_{k \neq i} {v}_{i} {v}_{k} [ \int \int {\int }_{Ω} {\sum }_{j} {( {σ}^{j} )}^{2} {g}_{{x}^{′} i}^{j} {g}_{{x}^{′} k}^{j} ]\end{matrix}} & \end{matrix}$$

Equation (A1) can be rearranged in matrix form as:

$$\begin{matrix}{max}_{{v}_{i}} \text{:} \frac{{{V}^{′}}^{T} C {V}^{′}}{{{V}^{′}}^{T} {C}_{0} {V}^{′}} & ( A2 ) \\ where , & \\ {V}^{′} = {( {v}_{1} {v}_{2} … {v}_{i} … {v}_{N} )}^{T} & \\ {C}_{i k} = \int \int {\int }_{{Ω}_{T}} {\sum }_{j} ( {( {σ}^{j} )}^{2} {g}_{{x}^{′} i}^{j} {g}_{{x}^{′} k}^{j} ) & \\ {C}_{0 i k} = \int \int {\int }_{Ω} {\sum }_{j} ( {( {σ}^{j} )}^{2} {g}_{{x}^{′} i}^{j} {g}_{{x}^{′} k}^{j} ) & \end{matrix}$$

Equation (A2) represents a quadradic optimization problem.

∇f_{\acute{x}i}=(g_{\acute{x}i}¹g_{\acute{x}i}²g_{\acute{x}i}³)^{T} represents the electric field E=(EₓE_{y}E_{z})^{T} in the domain Ω.

The elements Cᵢₖ of the matrix C and C₀ᵢₖ of the matrix C₀ can be calculated from data created in the performance of one or more stimulation waveform simulations (e.g. simulation data stored in library 16 as described herein).

Equation (A1) considers the integration of the square of the current magnitude. In some embodiments, the integration of square of the voltage magnitude can be performed (e.g. performed by algorithm 15). In some embodiments, the integration of electrical power can be performed.

The formulation described hereabove considers the ratio of integration of square of current density in the target region Ω_{T} with respect to integration of square of current density in the complete domain. In some embodiments, the weighted integration of square of current density in the complete domain for the numerator can be considered. The weight will be uniformly decaying away from the target point. In some embodiments, this weight can be considered to be decaying away from a cylindrical target region.

### Section 16—PNS Optimization Problem

The solution of the optimization problem discussed in Section 15 hereabove yields the voltage values for all stimulation elements 260. The current values can be determined from these voltage values using the conductance matrix (e.g. as discussed in Section 17 herebelow). However, it can be seen from equation (A2) that, changing the scale of the voltage or current does not change the maxima. Hence, the ratio of current values across stimulation elements 260 is a driving factor, not the absolute magnitude.

The optimization problem defined in equation (A2), repeated below,

$${max}_{{v}_{i}} \text{:} \frac{{{V}^{′}}^{T} C {V}^{′}}{{{V}^{′}}^{T} {C}_{0} {V}^{′}}$$

is a quadratic optimization problem. This formulation has no constraint on the number of current sources. It also does not restrict a stimulation element 260 from being a negative current source.

It can be seen that scaling \acute{V} does not change the value of the objective function. Thus, without loss of generality, a scale can be chosen such that:

\acute{V}^{T}C₀\acute{V}=1

Hence, the optimization problem is now a constrained optimization problem,

$${max}_{{v}_{i}} : {{V}^{′}}^{T} C {V}^{′} \qquad ( A3 )$$

which is subject to:

\acute{V}^{T}C₀\acute{V}=1

This constrained optimization method can be solved using the method of Lagrangian multipliers. Considering the Lagrangian function,

L(\acute{V}, λ)=\acute{V}^{T}C\acute{V}−λ(\acute{V}^{T}C₀\acute{V}−1)

the solution is given by evaluating the derivative of the Lagrangian function with respect to \acute{V}.

∇_{\acute{V}}L=2C\acute{V}−2λC₀\acute{V}=0

C\acute{V}=λC₀\acute{V} (A4)

Equation (A4) is a generalized eigenvalue problem. The eigenvalues will represent the value of the optimization function and the eigenvectors will represent the solutions \acute{V}. As an example, considering the maximum eigenvalue λₘₐₓ and the corresponding eigenvector \acute{V}ₘₐₓ, and substituting in the optimization function given in (A3), the following results:

\acute{V}ₘₐₓ^{T}C\acute{V}ₘₐₓ

.=\acute{V}ₘₐₓ^{T}(C\acute{V}ₘₐₓ₎

.=\acute{V}ₘₐₓ^{T}(λₘₐₓC₀\acute{V}ₘₐₓ)

.=λₘₐₓ(\acute{V}ₘₐₓ^{T}C₀\acute{V}ₘₐₓ)

.=λₘₐₓ

### Section 17—PNS Optimization Problem With Floating Stimulation Elements 260

A stimulation element 260 having no current sourcing or sinking through it, is categorized as a floating stimulation element 260, as described herein. In some embodiments, one or more stimulation elements 260 (e.g. present on one or more implanted leads 265), are configured to be floating (e.g. for all or a portion of a stimulation program to be delivered to the patient). In some embodiments, the number of “active” (non-floating) stimulation elements 260 is restricted, such as to perform desired current steering. In some embodiments, lead 265 and/or another component of delivery device 200 may have a hardware issue (e.g. a broken wire or trace), and one or more stimulation elements 260 can be configured to be floating to properly adjust to the hardware issue (e.g. avoid an undesired condition). In these and other situations in which one or more stimulation elements is configured as floating, algorithm 15 can solve the optimization problem based on those stimulation elements being floating (e.g. not able to source and/or sink current).

In Section 16, an optimization problem without any constraints on current sources was presented. In Section 17, the constraint of one or more stimulation elements configured as floating is addressed.

Considering the current-voltage relationship through the conductance matrix Y,

Í=Y\acute{V}

Í is a vector specifying currents flowing through each stimulation element 260. A positive value in the vector indicates an amount of current sourced from a stimulation element 260, and a negative value indicates an amount of current sunk into a stimulation element 260. The Y matrix is a square matrix with dimension equal to the number of stimulation elements 260 (N).

As described herein, current does not source or sink through any stimulation element 260 configured as floating. This zero-current condition is equivalent to setting the corresponding entries in 1 to zero. Letting the corresponding sub-matrix of Y be Y_{f},

\acute{0}=Y_{f}\acute{V} (A5)

Assuming m stimulation elements 260 to be floating, then Y_{f} will be a (N−m×N) matrix. Considering the singular value decomposition (SVD) of the matrix Y_{f},

Y_{f}=AΣB^{T}

the last m columns of Σ will be zeros. Also, B is an orthonormal matrix, therefore considering \acute{V} to be the linear combination of the last m columns of B will satisfy equation (A5). The sub-matrix formed by these last m columns can be defined as Bₛ=B(1:N,N−m+1: N). Then the linear combination of these columns can be represented as,

\acute{V}=Bₛα (A6)

Substituting equation (A6) in the optimization problem results in:

$${max}_{{v}_{i}} \text{:} \frac{{{V}^{′}}^{T} C {V}^{′}}{{{V}^{′}}^{T} {C}_{0} {V}^{′}} \qquad ( A7 )$$

$${max}_{α} \frac{{α}^{T} {B}_{s}^{T} C {B}_{s} α}{{α}^{T} {B}_{s}^{T} {C}_{0} {B}_{s} α} = \frac{{α}^{T} D α}{{α}^{T} {D}_{0} α}$$

$$subject to \text{:}$$

$${{V}^{′}}^{T} {C}_{0} {V}^{′} = {α}^{T} {D}_{0} α = 1$$

$${Y}_{f} {V}^{′} = 0$$

Equation (A7) represents another constrained quadratic optimization problem, which can be solved using the method of Lagrangian multipliers as described hereabove. Solving this optimization problem is equivalent to solving the generalized eigenvalue problem,

Dα=λD₀α

The maximum eigenvalue (λₘₐₓ) will be the maximum value of the optimization function and the corresponding eigenvector αₘₐₓ will represent the optimum value of the linear combiners. The contact voltage values will be given by:

Vₘₐₓ=Bₛαₘₐₓ

### Section 18—PNS Mixed Formulation

In some embodiments, algorithm 15 can compensate for one or more hardware restrictions in determining a stimulation paradigm SP in which current is steered toward one or more target regions Ω_{T}. Examples of hardware restrictions can include a maximum number of stimulation elements 260 available for sourcing and/or sinking current (e.g. due to a hardware issue as described herein). In some embodiments, in order to reduce hardware complexity of delivery device 200, only positive sources are provided by the hardware. All restrictions present need to be considered by algorithm 15 when solving a current steering optimization problem. Section 18 describes a current steering formulation which considers such restrictions in terms of additional constraints to the optimization problem.

The optimization problem formulated in Section 17 considers all stimulation elements 260 as active (non-floating, such as when sourcing current, sinking current, and/or functioning as a ground). In some embodiments, such as due to hardware restrictions, a delivery device 200 can have a maximum of four stimulation elements 260 that can be configured as positive current sources. In these embodiments, at least one stimulation element 260 can be configured to sink current, such as to define a reference potential. Stimulation elements 260 which are not configured as positive sources are to be considered either as being configured to be floating and/or to sink current.

In some embodiments, such as to reduce computational complexity, a maximum of eight stimulation elements 260 that are nearest to the target location can be considered for optimization by algorithm 15, and any remaining stimulation elements 260 can be assumed to be floating. In these embodiments, the following constraints can be used for the eight “active” stimulation elements 260: (1) at most four stimulation elements 260 source current (Iᵢ>0); (2) at least one stimulation element 260 is grounded (Vᵢ=0); and (3) the remaining stimulation elements 260 are configured to sink current and/or to be floating (Vᵢ=0 or Iᵢ=0). Considering the eight active stimulation elements 260, the possible element 260 combinations from these constraints are:

.=□⁸C₄(2⁴−1)+□⁸C₃(2⁵−1)+□⁸C₂(2⁶−1)+□⁸C₁(2⁷−1)

.=1050+1736+1764+1016

.=5566

In the first term, □⁸C₄ gives the total number of ways in which four stimulation elements 260 can be selected as current sources out of the eight elements 260. 2⁴−1 gives the total number of possible combinations of setting stimulation elements 260 as ground. Apart from source and ground, the remainder of stimulation elements 260 will be considered as floating. The second term uses the same logic for counting the combinations but with three current sources. The total 5566 combinations correspond to different configurations for which optimization needs to be performed. Each configuration specifies a mixture of sourcing, ground and floating constraints of the stimulation elements 260 for optimization.

In some embodiments, in an arrangement including two leads 265, each having eight stimulation elements 260, all 16 stimulation elements 260 can be considered while maintaining the constraints of sourcing, ground and floating elements 260.

In some embodiments, only anodic current sources are considered by algorithm 15 for stimulation elements 260 configured to source current. In other embodiments, only cathodic current sources are considered by algorithm 15 for stimulation elements 260 configured to source current. Considering only one of anodic or cathodic sources simplifies and reduces hardware requirements of delivery device 200.

### Constraints on Current Sources

As described hereabove in Section 17, floating stimulation element 260 (Iᵢ=0) constraints can be accommodated in the generalized eigenvalue formulation. In order to add the constraint Iᵢ≥0 for a selected set of stimulation elements 260, the formulation can be modified as follows:

$${max}_{α} \text{:} \frac{{α}^{T} D α}{{α}^{T} {D}_{0} α}$$

$$subject to \text{:}$$

$${Y}_{s} {B}_{s} α \geq {0}^{′}$$

where Yₛ is a sub-matrix of conductance matrix Y. Yₛ is constructed by selecting rows in the Y matrix corresponding to the selected current sources (Iᵢ≥0).

In the above formulation, the value of the scale of the solution to the optimization problem α does not change the value of the objective function. Furthermore, if an a is in a feasible region, then any positive scale of α also lies in the feasible region. Without loss of generality, a positive scale of α can be chosen such that α^{T}D₀α=1. Thus, the above optimization problem can be restated as:

$${max}_{α} \text{:} {α}^{T} D α$$

$$subject to \text{:}$$

$${a}^{T} {D}_{0} α = 1$$

$${Y}_{s} {B}_{s} α \geq {0}^{′}$$

The above formulation is a linearly constrained generalized eigenvalue problem. Since the constraints above are not convex, the problem cannot be solved using convex optimization methods. A solution can be found by using Sequential Quadratic Programming (SQP), an iterative method for constrained nonlinear optimization. Note that the iterative solver can get stuck in a local maxima.

### Constraints on Stimulation Elements 260 Configured to Sink Current

In some embodiments, algorithm 15 configures at least one stimulation element 260 (e.g. of eight elements 260) to sink current (Vᵢ=0). Setting a selected set of stimulation elements 260 to ground adds an equality constraint (Vᵢ=0) to the optimization formulation. The above optimization formulation is derived from the optimization formulation in terms of voltage vector V. The equality constraint will set the selected components of the vector V to zero in the optimization formulation of equation (A3). Thus, the constraint Vᵢ=0 reduces the dimensionality of the optimization problem. The linearly constrained eigenvalue problem can be easily derived from (A3) by setting the selected components of Vᵢ=0.

The linearly constrained eigenvalue problem is solved for 5566 different stimulation element 260 combinations. The solution corresponding to the maximum objective function value is chosen by algorithm 15 as the final solution. The corresponding eigenvector αₘₐₓ, will represent the optimum value of the linear combiners. The corresponding stimulation element 260 combination will be the final element 260 strategy for optimum current steering.

The corresponding contact voltage values is defined as:

\acute{V}ₘₐₓ=Bₛαₘₐₓ

The corresponding contact current values is defined as:

Íₘₐₓ=YₛBₛαₘₐₓ

### Optimal Solution for Mixed Constrained Optimization

The floating stimulation element 260 formulation described in Section 17 used the singular value decomposition for expressing the voltage as Bₛα and the corresponding currents as YₛBₛα. Bₛ gives a basis for voltage vector V such that it satisfies the floating element 260 constraint. Another way to select Bₛ is such that YₛBₛ is an identity matrix and Y_{f}Bₛ=0. Such a Bₛ can be found using Q-R decomposition of the matrix [Yₛ^{T}, Y_{f}^{T}] and selecting initial columns equal to number of sources. This changes the optimization problem to:

YₛBₛα≥\acute{0}

α≥\acute{0}

Then the optimization problem can be defined as:

$${max}_{α} \text{:} {α}^{T} D α \\ subject to \\ {α}^{T} {D}_{0} α = 1 \\ α \geq {0}^{′} \qquad ( A8 )$$

To solve this optimization problem, a Lagrangian function as follows can be considered:

L(α, λ, ω)=α^{T}Dₛα+λ(1−α^{T}D₀ₛα)+ω^{T}α

where λ(≥0) and ω (≥0) are the Lagrangian multipliers for the equality and inequality constraints respectively. The gradients of this Lagrangian function with respect to α, λ and ω are then equated to zero to get the solution of the constrained optimization problem:

∇_{α}L=Dₛα−λD₀ₛα+ω=\acute{0}

∇_{λ}L=1−α^{T}D₀ₛα=0

∇_{ω}L=α=\acute{0} (A9)

Considering the Karush-Kuhn-Tucker (KKT) complementary slackness conditions:

ωᵢ=0, αᵢ≥0

ωᵢ≥0, αᵢ=0

This effectively implies

ω^{T}α=0

In considering equation (A9), this can be rearranged as:

ω=λD₀ₛα−Dₛα=(λD₀ₛ−Dₛ)α

A subset of indices P ⊆ {1,2,3, . . . n} can be considered such that:

ω_{P}=\acute{0}, α_{P}≥\acute{0}

ω_{\acute{P}}≥\acute{0}, α_{\acute{P}}=\acute{0}

These equations can be represented as:

$$\{ \begin{matrix}{0}^{′} \\ {ω}_{{P}^{′}}\end{matrix} \} = \{ λ {D}_{0 s} - {D}_{S} \} \{ \begin{matrix}{α}_{P} \\ {0}^{′}\end{matrix} \}$$

which yields an equation in the sub-matrix of λD₀ₛ−Dₛ, which can be written as:

λD₀ₛ_{_{PP}}α_{P}−Dₛ_{_{PP}}α_{P}=\acute{0}_{P}

which is a generalized eigenvalue problem. An eigenvector α obtained by solving the generalized eigenvalue problem that also satisfies the following:

ω_{\acute{P}}≥0 (A10)

which is a solution of the optimization problem (A8). Solving the above generalized eigenvalue problem for each subset P that satisfies (A10) gives a local maxima. Selecting the solution that has the maximum eigenvalue among these local maxima gives the optimal solution for mixed constrained optimization. Since there are finitely many subsets P and there are finitely many eigenvalue solutions for each subset, the total number of local maxima is finite.

The solution of the optimization problem gives the relative ratio of current values for different stimulation elements 260. It is possible that multiple combinations of the elements 260 give the same solution of the optimization problem. In some embodiments, algorithm 15 compares such combinations and selects (e.g. as a stimulation paradigm SP for use) the combination with minimum number of stimulation elements 260 configured as active.

The optimization problem can be defined such that the integration of current density in the complete tissue domain Ω is unity. Hence, the solution of the optimization problem, such as to achieve optimized current steering, represents the integration of current density in the target region Ω_{T}. The total power provided by the stimulation elements 260 to achieve the solution of the optimization problem can be different for different focus locations (target regions Ω_{T}).

In some embodiments, the solution of the optimization problem for all target regions can be compared by algorithm 15 for a particular lead 265 configuration. As described herein, any scale of the current values can satisfy the optimization problem. The scale for every target region Ω_{T} can be selected such that the total power in the target region Ω_{T} is same for all locations of the target region Ω_{T} for a particular lead 265 configuration.

### Section 19—PNS—Determining Numerically Stable Solutions

The process of determining the optimum stimulation element 260 excitation pattern to achieve the desired current steering can involve algorithm 15 determining an optimization that is based on results from FEA simulations (e.g. as performed by a manufacturer of apparatus 10). Algorithm 15 can comprise an optimization solver that determines an optimized stimulation element 260 excitation pattern corresponding to the maximum value of the objective function. The results from FEA simulations (e.g. as stored in whole, in part, and/or in a summarized fashion in library 16 described herein) can be used by the optimization solver of algorithm 15. These FEA simulations can involve a large amount of numerical computations, and correspondingly can introduce minor numerical errors in the simulation results. Thus, it is necessary to confirm the stability of the optimized solution.

It is observed that there are multiple stimulation element 260 excitation patterns which have the objective function value within 5% of the maximum objective function value. This multiplicity enables a flexibility to explore the possibility of choosing between multiple solutions, in order to identify one or more numerically stable solutions which does not significantly compromise the objective function value.

As described in Section 18, the optimization solver of algorithm 15 can iterate over a finite number of numerous stimulation element 260 combinations. For each of these combinations, the optimization solver can evaluate the objective function value. A numerically stable solution can be selected among these finite combinations based on their excitation patterns and the objective function values. For finding the numerically stable solution, these combinations can be grouped, where each group has combinations having the same elements 260 as current sources and having objective function value above a certain threshold. A “numerically stable solution” is defined herein as belonging to the group with highest cardinality and having the minimum number of active stimulation elements 260 (elements 260 configured as source and/or ground contacts).

Algorithm 15 can be configured to determine a numerically stable solution after the optimization solver results are obtained.

In some embodiments, in a first step for determining a numerically stable solution, algorithm 15 defines a threshold for the objective function values. For example, the threshold can be 95% of the objective function value of the existing optimum stimulation element 260 excitation pattern. The stimulation element 260 combinations having objective function values that are more than the threshold are selected for further analysis.

In a second step, algorithm 15 checks the source contacts of all the combinations selected in the first step. Combinations having the same source contacts are grouped together.

The cardinality (number of elements) of all such groups is evaluated by algorithm 15. The group with the highest cardinality can be selected by algorithm 15 for further analysis.

In a third step, algorithm 15 checks all the contact combinations in the group selected in the second step. The quantity of active stimulation elements 260 can be evaluated for all these combinations. The combination with the minimum number of active contacts can then be considered as the numerically stable solution.

While algorithm 15 described hereabove in reference to Sections 16 through 19 has been described in terms of peripheral nerve stimulation (PNS), it can be applied to current steering of nerves of the spinal cord and other body locations as well.

### Section 20—User Interface

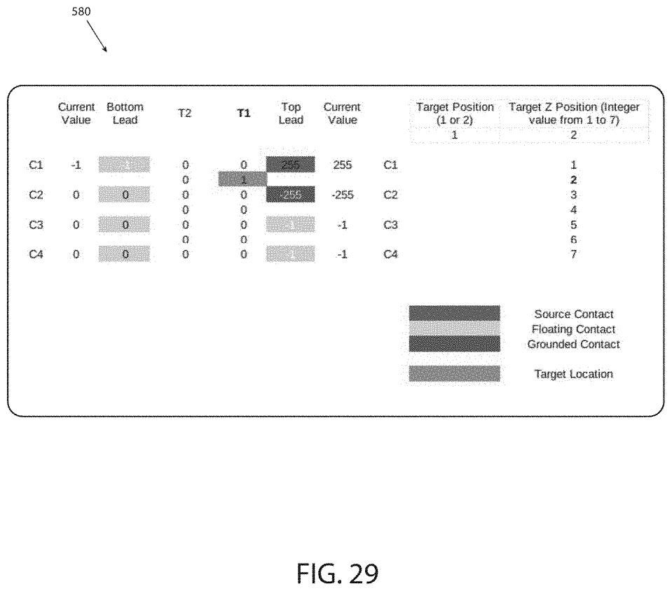

Referring now to FIG. 29, a user's view of a user interface of a stimulation system is illustrated, consistent with the present inventive concepts. Apparatus 10 can include one or more user interfaces, such as user interface 580 shown in FIG. 29 and described herein (e.g. a user interface of external device 500 and/or a user interface of programmer 600). User interface 580 can include one or more user input components (e.g. buttons, slides, knobs, and the like) and/or one or more user output components (e.g. lights, displays and the like). User interface 580 can be configured to allow an operator (e.g. a clinician) to select a location of a target region Ω_{T} into which stimulation energy can be delivered (e.g. into which stimulation current can be steered). For the selected target region Ω_{T} (e.g. a region proximate one or more peripheral nerves or one or more nerves of the spinal cord), the stimulation element 260 activation pattern can be represented by a graphical characteristic shown on user interface 580 (e.g. a color scale, or other variable graphical parameter). Alternatively or additionally, variable quantitative and/or qualitative information can be displayed on user interface 580. The relative ratio of current values can be normalized to a range of integer values, such as a range between 0 and 255.

User interface 580 can provide and/or receive (e.g. receive from an operator and/or from another component of apparatus 10) data related to a location of each lead 265 (e.g. a relative location as compared to another lead 265). User interface 580 can provide and/or receive data related to the configuration of each stimulation element 260 (e.g. information related to the element 260 being configured as a source, floating, or grounded).

Apparatus 10 can be configured to provide peripheral nerve stimulation (PNS) including both tonic stimulation waveforms, as well as more complex stimulation waveforms (e.g. as defined by stimulation paradigm SP). Experiments have shown that the amplitude and pulse width combinations required to activate motor nerves differ from those used to activate sensory nerves. For example, motor nerves may be preferentially activated using wide pulses and sensory nerves maybe activated using narrow pulses (e.g. at a given stimulus amplitude). Generally, the target of PNS is a sensory nerve, however in the implantation setting (e.g. an operating room) it may be necessary to target (e.g. stimulate) the motor nerve to confirm (e.g. if the patient is sedated) appropriate placement of lead 265 and its associated stimulation elements 260 (e.g. a visual confirmation of an appropriate muscle twitch).

Pulse width can have a significant effect on the preferential activation of nerve fibers with different diameters. For instance, a stimulation signal with a high amplitude may be necessary to activate narrow fibers whereas lower amplitude may be sufficient to activate larger fibers (e.g. at a given stimulus pulse width).

“Pre-pulses”, such as low amplitude pulses that precede one or more higher amplitude stimulation pulses, can be used to improve the selectivity and efficiency of the succeeding stimulating pulses (e.g. by hyperpolarization or similar means).

Apparatus 10 can be configured to delay charge recovery by using an inter-phase gap, as described herein, to improve the efficiency of delivered stimulation pulses.

Delivery of a stimulation pulse causes action potentials to propagate in both orthodromic and antidromic directions. Depending on the desired effect of the stimulation, one of these propagations may be the preferred activation and the other may be a side-effect. The undesired action potential may be arrested with a carefully placed anodic pulse that is delivered by delivery device 200 (e.g. by a stimulation element 260) at a time when the action potential may be propagating past it. It is important that such an “arrest pulse” does not generate action potentials of its own, “secondary action potentials” herein. The shape of the arrest pulse delivered by delivery device 200 can be configured to avoid generating these secondary action potentials, for example, the shape of the arrest pulse can be triangular, trapezoidal, and/or quasi-trapezoidal. In some embodiments, the arrest pulse is combined with an asymmetric active recovery pulse. Alternatively or additionally, passive recovery can be implemented.

Evoked compound action potential (eCAP) is a measure of the depolarization of a group of neurons. Apparatus 10 can be configured to use eCAP as an indication of the distance of the lead 265 (e.g. of one or more stimulation elements 260) to the target neurons (e.g. to target region Ω_{T}). In some embodiments, apparatus 10 provides a display (e.g. a real time or “live” display), such as via user interface 580, of eCAP magnitude during lead 265 (e.g. stimulation element 260) implantation (e.g. to aid in the insertion of lead 265 into the patient).The display of eCAP magnitude can be used to determine the latency of a propagating eCAP. Alternatively or additionally, eCAP magnitude can be used to apply an arrest pulse.

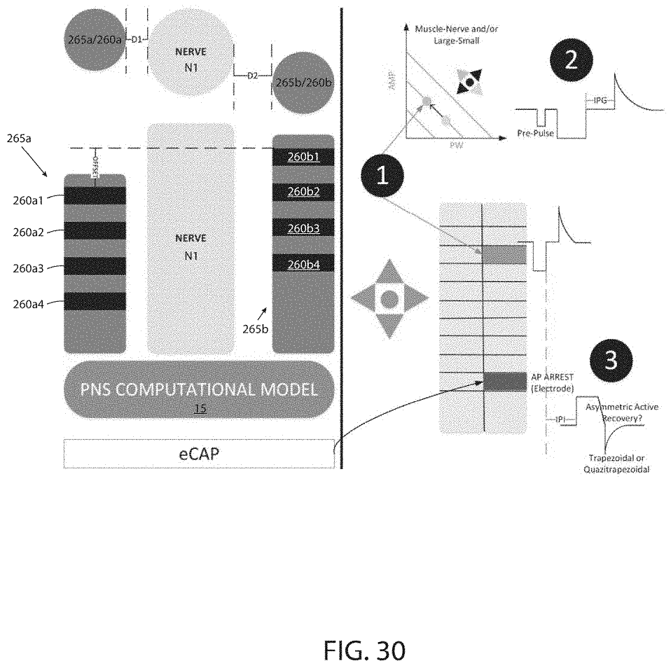

Referring now to FIG. 30, a schematic view of an implantation arrangement and a three-step workflow is illustrated, consistent with the present inventive concepts. As described hereabove, FEA techniques can be used to focus stimulation on different segments of the nerve. A clinician can provide information related to algorithm 15 (e.g. an FEA algorithm), such as the location of one or more leads 265 relative to one or more nerves to be stimulated, nerve N1 shown. This information may be conveyed by observing images from an imaging device of apparatus 10 (e.g. an ultrasound imager, fluoroscope, X-ray, and the like). Algorithm 15 can include an image processing algorithm that can automatically determine the distance between the lead 265 and target nerve N1. The orientation between the lead 265 and the nerve N1 can also be determined, such as is described in applicant's co-pending International PCT Patent Application Serial Number PCT/US2020/066901, titled “Systems with Implanted Conduit Tracking”, filed Dec. 23, 2020 [Docket nos. 47476-716.601; NAL-022-PCT]. In some embodiments, algorithm 15 uses eCAP magnitude and propagation delay to assess the distances between lead 265 and the target one or more nerves, nerve N1 shown.

In a first step, algorithm 15 can generate a computation model (or precomputed model data can be generated) to determine the source, sink and floating stimulation elements 260 to steer current to focus the stimulation locus (also referred to as “centroid”) on a desired location (e.g. target region Ω_{T}). The desired location can be determined by obtaining paresthesia feedback from the patient, and/or it can be selected based on non-patient-feedback criteria (e.g. anatomic targets, physiological markers, and the like). The stimulation waveform delivered by delivery device 200 can be a biphasic waveform (e.g. including active and/or passive recovery pulses). In some embodiments, delivery device 200 provides one or more stimulation waveforms as described in applicant's co-pending International PCT Patent Application Serial Number PCT/US2020/066901, titled “Systems with Implanted Conduit Tracking”, filed Dec. 23, 2020 [Docket nos. 47476-716.601; NAL-022-PCT].

At a given target location (e.g. target region Ω_{T}), a user (e.g. clinician) could traverse a pulse width-amplitude (PW-AMP) line to target muscle versus nerve fiber, and/or large diameter versus small diameter fiber. Traversing along a line would provide constant charge, while moving up or down to adjacent lines would provide more or less charge.

In a second step, once an appropriate locus and strength has been determined, a pre-pulse can be added to the stimulation waveform (e.g. as defined by stimulation paradigm SP). The associated charge (amplitude times pulse width) in the pre-pulse as well as temporal proximity to the stimulation pulse can be adjusted, such as to “fine tune” the sensation (e.g. paresthesia) and/or pain relief. The inter-phase gap can then be adjusted to enhance the efficiency of the pulse.

In a third step, if secondary stimulation or other side effects are detected, using eCAP measurements (e.g. in an implantation or other intra-operative procedure, during a stimulation trialing procedure, and/or with a delivery device 200) one or more arrest pulses can be delivered. An arrest pulse is likely to be applied via a most proximal or most distal stimulation element 260, although it can be delivered by an element 260 at any location along the length of lead 265. The amplitude of the arrest pulse can be maintained such that the side-effect is eliminated without generating additional paresthesia or other sensations.

While the preferred embodiments of the devices and methods have been described in reference to the environment in which they were developed, they are merely illustrative of the principles of the present inventive concepts. Modification or combinations of the above-described assemblies, other embodiments, configurations, and methods for carrying out the invention, and variations of aspects of the invention that are obvious to those of skill in the art are intended to be within the scope of the claims. In addition, where this application has listed the steps of a method or procedure in a specific order, it may be possible, or even expedient in certain circumstances, to change the order in which some steps are performed, and it is intended that the particular steps of the method or procedure claim set forth herebelow not be construed as being order-specific unless such order specificity is expressly stated in the claim.

1.-22. (canceled)

23. A system for delivering stimulation energy to a patient comprising:
a controller;
a memory coupled to the controller and storing instructions for the controller to perform an algorithm; and
one or more leads for implantation inside a human body, wherein each lead comprises a plurality of stimulation elements,
wherein each stimulation element can be in one of at least two configuration states,
wherein a first configuration state comprises a stimulation element sourcing and/or sinking current,
wherein a second configuration state comprises a stimulation element being in an electrically passive state, and
wherein the algorithm is configured to determine a stimulation paradigm for stimulating one or more target locations of a patient, wherein the stimulation paradigm defines the configuration state of each of the plurality of the stimulation elements of a first lead.

24. The system of claim 23, wherein the algorithm is configured to determine the stimulation paradigm that preferentially stimulates a target region by maximizing the ratio of the integral of the square of the current density in the target region to the integral of the square of the current density in a large part of the human body.

25. The system of claim 24, wherein the algorithm determines the stimulation paradigm by solving one or more generalized eigenvalue problems.

26. The system of claim 24, wherein the algorithm does not match a specific current or voltage distribution through an entire domain.

27. The system of claim 23, wherein the stimulation paradigm further defines the configuration state of each of the plurality of the stimulation elements of a second lead.

28. The system of claim 23, wherein the plurality of stimulation elements of each lead comprises two or more stimulation elements, wherein the algorithm is configured to cause at least one of the stimulation elements of each lead to source and/or sink current, and wherein the algorithm is further configured to cause the remaining stimulation elements of each lead to be in a floating state.

29. The system of claim 23, wherein the first configuration comprises:
a first arrangement in which a first set of one or more stimulation elements each deliver a particular current; and/or a second arrangement in which a second set of one or more stimulation elements are each set to a particular voltage.

30. The system of claim 29, wherein the first arrangement comprises the first set of stimulation elements each delivering a positive or zero current, and the second arrangement comprises the second set of stimulation elements each being set to ground voltage.

31. The system of claim 29, wherein the first arrangement comprises the first set of stimulation elements each delivering a negative or zero current, and the second arrangement comprises the second set of stimulation elements each being set to ground voltage.

32. The system of claim 29, wherein no greater than four of the stimulation elements of each of the one or more leads are configured in the first arrangement.

33. The system of claim 23, wherein the algorithm is configured to steer current using a finite element analysis technique.

34. The system of claim 23, wherein the algorithm utilizes one or more constraints to determine the stimulation paradigm.

35. The system of claim 34, wherein the algorithm limits the number of stimulation elements that are configured as a source and/or a sink.

36. The system of claim 23, wherein the algorithm determines the stimulation paradigm based on one or more of the stimulation elements being classified as open and/or shorted.

37. The system of claim 23, further comprising a library of predetermined information, wherein the algorithm is configured to determine the stimulation paradigm based on the predetermined information.

38. The system of claim 37, wherein the algorithm is further configured to correlate implant locations and/or implant geometries of the one or more leads to the predetermined information.

39. The system of claim 23, further comprising a user interface configured to allow an operator to specify a location into which current delivered by the stimulation elements can be steered.

40. The system of claim 23, wherein the algorithm is configured to determine the stimulation paradigm using an inverse solution.

41. The system of claim 40, wherein the inverse solution predicts an anatomical location to be stimulated based on a given stimulation paradigm.

42. The system of claim 41, wherein the given stimulation paradigm comprises a set of stimulation elements to be configured as anodes and/or cathodes, as well as a set of current amplitudes to be delivered to each stimulation element.

43. The system of claim 23, wherein the algorithm is configured to steer current to a target location in the patient's spinal cord.

44. The system of claim 23, wherein the algorithm is configured to steer current to a target location in the patient's peripheral nervous system.
